(19) United States
(12) Patent Application Publication
(10) Pub. No.: US 2025/0134875 A1
(43) Pub. Date: May 1, 2025

(54) FORMULATIONS OF ALK-5 KINASE INHIBITORS AND USES THEREOF

(71) Applicant: Thirona Bio, Inc., San Diego, CA (US)

(72) Inventors: Kieran George Mooney, Lyme, CT (US); John Gordon Foulkes, Rancho Santa Fe, CA (US); David A. Bullough, Encinitas, CA (US)

(73) Assignee: Thirona Bio, Inc., San Diego, CA (US)

(21) Appl. No.: 18/923,985

(22) Filed: Oct. 23, 2024

Related U.S. Application Data
(60) Provisional application No. 63/694,737, filed on Sep. 13, 2024.
(60) Provisional application No. 63/593,166, filed on Oct. 25, 2023.

(51) Int. Cl. A61K 31/444; A61K 9/00; A61K 9/06; A61K 47/10; A61K 47/12; A61K 47/14; A61K 47/18; A61K 47/26; A61K 47/32; A61K 47/34; A61K 47/38; A61K 47/44; A61P 17/02

(52) U.S. Cl. CPC A61K 31/444; A61K 9/0014; A61K 9/06; A61K 47/10; A61K 47/12; A61K 47/14; A61K 47/18; A61K 47/26; A61K 47/32; A61K 47/34; A61K 47/38; A61K 47/44; A61P 17/02

(57) ABSTRACT

Compositions, methods of making and methods of using are provided for topical administration of pharmaceutical compositions comprising one or more activin receptor-like kinase-5 (ALK-5) kinase inhibitors. The pharmaceutical compositions can be used for treatment of diseases and disorders affecting the skin.

### FIELD OF THE INVENTION

The invention relates to pharmaceutical compositions of activin receptor-like kinase-5 (ALK-5) kinase inhibitors, the process of preparing these compositions, and methods of using these compositions for treatment of diseases and disorders by application to the skin.

### BACKGROUND OF THE INVENTION

Transforming growth factor (TGF)-β receptors activate fibrotic and tumor-promoting signaling cascades. Three mammalian TGF-βs, TGF-β₁, TGF-β₂, and TGF-β₃, can activate the TGF-β pathway. The three TGF-βs share over 80% sequence identity, bind to the same receptor system (TGF-β1 receptor), and utilize the same signal transduction mechanisms (Vander Ark, 2018). However, they have different promoter regions and show cell type specific expression. TGF-β first binds to a type II receptor (TβRII), which then binds to and phosphorylates a type I receptor (TβRI) (i.e., an activin receptor-like kinase (ALK)). There is a family of ALK proteins including ALK-5, which is the most specific ALK for TGF-β. Activation of ALK-5 leads to phosphorylation of intracellular proteins, including phosphorylation of SMAD transcription factors (amongst others), which in turn upregulate a pro-fibrotic response involving multiple genes (Aashaq 2022; Cui 2019). There also are SMAD-independent TGF-β signaling processes, but they too are controlled by the ALK-5 kinase domain (Aashaq 2022). Thus, inhibiting ALK-5 kinase has the potential to block excess fibrosis driven by this mechanism.

Human skin is composed of three outer layers; the outer stratum corneum, below which lies the epidermis; and below that the dermis. Following any injury to human skin, which impacts down through to the dermis, the normal physiological response to wound healing is to rapidly close the lesion to prevent blood loss, minimize infection and locally repair the damaged tissue. TGF-β plays multiple roles in that process including chemoattraction of various cell types into the wound; cell proliferation and differentiation of fibroblasts into myofibroblasts to facilitate wound closure; and stimulation of collagen production and other extracellular matrix (ECM) proteins. This provides a framework of granulation tissue to bridge the wound and allow new vascular in-growth. In evolution, the goal of skin fibrosis was simply rapid wound closure. Skin scarring, which could be viewed as skin fibrosis in its simplest form, is the result of excessive ECM production in the dermis and can be considered the product of abnormal wound healing. While clinical efficacy remains largely elusive, a broad range of anti-TGF-β therapies have shown efficacy across multiple animal models of fibrosis including TGF-β antibodies, TGF-β ligand traps and inhibitors of TGF-β receptor kinases (Li 2021), with multiple reports on anti-fibrotic activity with anti-TGF-β therapies in the skin (Liarte 2020, Boys et al 2012, Bian 2011).

One example of a condition that involves fibrosis is the formation of scars during wound repair. Scars, including hypertrophic and keloid scars, typically result from the deposition of collagen at wound sites. Wounds may be produced through many different kinds of mechanisms including surgery, accidental injuries, burns, trauma, etc. It has been reported that the application of TGF-β₃, antibodies to TGFβ₁ and TGFβ₂, which inhibit the TGF-β pathway, can assist in reducing scarring (O'Kane and Ferguson, (1997) Int. J. Biochem. Cell Biol., 29: 63-78). Accordingly, small molecule ALK-5 kinase inhibitors may be administered to reduce scar formation, and for the treatment of other fibrotic conditions, as well as skin cancers.

The present invention provides a pharmaceutical composition including a potent, locally-acting drug to inhibit ALK-5 kinase for the treatment of skin and other diseases and disorders by application to the skin. Safety is achieved by minimizing systemic exposure of the ALK-5 kinase inhibitor to extremely low levels and by using inhibitors designed to be rapidly metabolized in the liver, thereby minimizing the potential risk of systemic inhibition.

### SUMMARY OF THE INVENTION

The invention is directed to pharmaceutical compositions of ALK-5 kinase inhibitors for treatment of various diseases and disorders related to the TGF-β receptor. ALK-5 kinase is also known as TGF-β type 1 receptor kinase. The diseases and disorders to be treated by the compositions of the invention are on the skin, or affect the skin, or can be affected by application to the skin and, as such, the current invention focuses on topical formulations of ALK-5 kinase inhibitors. In particular, the invention recognizes that there is a need for topical formulations comprising ALK-5 kinase inhibitors where the formulations provide sufficient dermal exposure of the ALK-5 kinase inhibitors within the skin. In addition, the formulations of the invention do not result in a high systemic exposure to ALK-5 kinase inhibitors. Since high systemic exposure of ALK-5 kinase inhibitors may have adverse effects, the formulations of the invention are designed to reduce systemic exposure to ALK-5 kinase inhibitors.

The topical pharmaceutical compositions of the invention have several beneficial properties. These beneficial properties, include, but are not limited to: (i) optimal solubility of the one or more ALK-5 kinase inhibitors in the formulation, (ii) high permeability of the one or more ALK-5 kinase inhibitors into the skin tissues, especially those of the dermis, (iii) low systemic exposure of the one or more ALK-5 kinase inhibitors included in the formulation, (iv) shelf stability of the pharmaceutical composition, (v) low skin irritation caused by the pharmaceutical composition by the one or more ALK-5 kinase inhibitors or any other excipient in the formulation, (vi) optimal dermal deposition of one or more ALK-5 kinase inhibitors as compared to the systemic exposure of the drug, (vii) sustained exposure of one or more ALK-5 kinase inhibitors in the skin and at levels leading to optimal therapeutic benefit, and (ix) ease of application of the drug with optimal spreading, absorption and cosmesis properties on the skin. The pharmaceutical compositions of the invention may have one or more of these properties.

In particular, the invention recognizes that for compositions to be used by patients such as, for example, scleroderma patients, the pharmaceutical compositions should be moisturizing, moderately cooling and/or non-oily/sticky. These pharmaceutical compositions of the invention should be optimal therapeutically and cosmetically as the patients suffering from, for example, scleroderma may have highly sensitive skin and the application of compositions on the affected area may be often painful for the patient(s). In addition, for treatment of patients suffering from, for example, scleroderma and/or keloid scars, the pharmaceutical composition should have the ability to cover the affected areas effectively and to rub in rapidly. Thus, in one aspect, the pharmaceutical compositions of the invention are moisturizing, moderately cooling and/or non-oily/sticky. In certain embodiments, these compositions are easily spreadable to cover the affected areas effectively.

In one aspect, the pharmaceutical compositions of the invention comprise: at least one ALK-5 kinase inhibitor; a permeation enhancer; a solvent; an antioxidant; a thickening agent, and optionally a preservative. In certain embodiments, the pharmaceutical compositions of the invention may not include a preservative. In certain embodiments, the pharmaceutical compositions of the invention may include a preservative.

In one aspect, the invention provides a pharmaceutical composition for topical application, the pharmaceutical composition comprising: one or more ALK-5 kinase inhibitors; a permeation enhancer; a solvent; a preservative; an antioxidant; and a thickening agent. In certain embodiments, the pharmaceutical composition is a cream or a topical gel. In certain embodiments, the pharmaceutical composition is a cream. In certain embodiments, the pharmaceutical composition is a topical gel. In certain embodiments, the pharmaceutical composition comprises a therapeutically effective amount of the ALK-5 kinase inhibitor. In certain embodiments, the pharmaceutical composition comprises from about 0.05% (w/w) to about 5% (w/w) of the ALK-5 kinase inhibitor. In preferred embodiments, the pharmaceutical composition comprises from about 0.05% (w/w) to about 1% (w/w) of the ALK-5 kinase inhibitor. In certain embodiments, the pharmaceutical composition comprises from about 0.08% (w/w) to about 1.3% (w/w) of the ALK-5 kinase inhibitor. In certain embodiments, the pharmaceutical composition comprises from about 0.1% (w/w), about 0.3% (w/w), or about 1% (w/w) of the ALK-5 kinase inhibitor.

Preferably, the ALK-5 kinase inhibitor is a small molecule inhibitor. The ALK-5 kinase inhibitor may be any of the ALK-5 kinase inhibitor molecules disclosed in any of: U.S. Pat. No. 7,964,612 (incorporated by reference in its entirety), U.S. Pat. No. 8,455,512 (incorporated by reference in its entirety), U.S. Pat. No. 9,938,289 (incorporated by reference in its entirety), U.S. Pat. No. 9,090,625 (incorporated by reference in its entirety), and/or U.S. Pat. No. 9,260,450 (incorporated by reference in its entirety).

In certain embodiments, the pharmaceutical composition achieves high dermal penetration to enable increased delivery of the ALK-5 kinase inhibitor from the composition into the skin when the pharmaceutical composition is applied to the skin. In certain embodiments, the pharmaceutical composition achieves low dermal absorption to increase dermal deposition and reduce levels of the ALK-5 kinase inhibitor in the systemic circulation. In certain embodiments, the pharmaceutical composition is stable under storage conditions.

In certain embodiments, the thickening agent in the pharmaceutical composition is selected from the group consisting of: carbomer, methyl cellulose, sodium carboxyl methyl cellulose (NaCMC), carrageenan, colloidal silicon dioxide, trolamine, guar gum, hydroxypropyl cellulose (HPC), hydroxypropyl methyl cellulose (HPMC), gelatin, polyethylene oxide, alginic acid, sodium alginate, fumed silica, and any combination thereof. In certain embodiments, the pharmaceutical composition comprises from about 0.5% (w/w) to about 10% (w/w) of the thickener. In certain embodiments, the pharmaceutical composition comprises from about 1% (w/w) to about 5% (w/w) of the thickener. In certain embodiments, the pharmaceutical composition comprises about 2% (w/w) of the thickener.

In certain embodiments, the antioxidant in the pharmaceutical composition is selected from a group a consisting of: butylated hydroxyanisole, butylated hydroxytoluene, tocopherol, propyl gallate, vitamin E, tert-butylhydroquinone and a combination thereof. In certain embodiments, the pharmaceutical composition comprises from about 0.05% (w/w) to about 0.5% (w/w) of the antioxidant. In certain embodiments, the pharmaceutical composition comprises about 0.2% (w/w) of the antioxidant.

In certain embodiments, the pharmaceutical composition of the invention is stable without the addition of a preservative. Thus, in those embodiments, the pharmaceutical compositions of the invention do not include a preservative.

The invention further provides embodiment where preservatives may be included. In certain embodiments, the preservatives of the invention may be an antimicrobial preservative. In certain embodiments, the preservative in the pharmaceutical composition is selected from a group consisting of: benzyl alcohol, imidazolidinyl urea, diazolidinyl urea, dichlorobenzyl alcohol, chloroxylenol, methyl paraben, ethyl paraben, propyl paraben, butyl paraben, phenoxyethanol, sorbic acid, benzoic acid, benzalkonium chloride, phenyl mercuric acetate, chlorobutanol, phenoxyethanol, and any combination thereof. In certain embodiments, the pharmaceutical composition comprises about 0.05% (w/w) to about 0.5% (w/w) of the preservative. In certain embodiments, the pharmaceutical composition comprises about 0.1% (w/w) to about 0.3% (w/w) of the preservative.

In certain embodiments, the solvent in the pharmaceutical composition is selected from a group consisting of water, hexylene glycol, propylene glycol, oleyl alcohol, propylene carbonate, mineral oil, diethylene glycol monoethyl ether, ethanol, polyethylene glycol, water, isopropanol, t-butyl alcohol, amyl alcohol, benzyl alcohol, diacetone alcohol, hexyl alcohol, tetrahydrofurfuryl alcohol, acetic acid, carboxylic acids including long chain fatty acids such as stearic and isostearic, 1,2-hexanediol, butylene glycol, diethylene glycol, dipropylene glycol, ethyl hexanediol, ethylene glycol, propylene glycol monolaurate, tetraethylene glycol, triethylene glycol, tripropylene glycol, butyl stearate, C₁₂₋₁₅ alkyl benzoate, C₁₂₋₁₅ alkyl lactate, caprylic/capric triglyceride, cetearyl ethylhexanoate, diethylhexyl adipate, di-ethylhexyl succinate, diisopropyl adipate, dioctyl malate, di-PPG-2 myreth-10 adipate, di-PPG-3 myristyl ether adipate, ethyl oleate, ethylhexyl cocoate, ethylhexyl hydroxystearate, ethylhexyl palmitate, ethylhexyl pelargonate, ethylhexyl stearate, hexyl laurate, hexyldecyl laurate, an any combination thereof. In certain embodiments, the pharmaceutical composition comprises about 30% (w/w) to about 98% (w/w) of the solvent. In certain embodiments, the pharmaceutical composition comprises about 40% (w/w) to about 95% (w/w) of the solvent.

In certain embodiments, the permeation enhancer in the pharmaceutical composition is selected from a group consisting of propylene glycol, ethanol, isopropyl alcohol, oleic acid, polyethylene glycol, diethylene glycol monoethyl ether, isopropyl myristate, dimethyl sulfoxide, capric acid, hexanoic acid, lauric acid, linoleic acid, linolenic acid, propionic acid, glycerol monolaurate, glycerol monooleate, glycerol monolinoleate, isopropyl isostearate, isopropyl palmitate, diethylsebacate, sorbitan monopalmitate, sorbitan oleate, sorbitan dilaurate, sorbitan trioleate, propylene glycol monolaurate, sucrose monolaurate, and any combination thereof. In certain embodiments, the pharmaceutical composition comprises from about 10% (w/w) to about 70% (w/w) of the permeation enhancer. In certain embodiments, the pharmaceutical composition comprises from about 20% (w/w) to about 60% (w/w) of the permeation enhancer. In certain embodiments, the pharmaceutical composition comprises from about 30% (w/w) to about 50% (w/w) of the permeation enhancer.

In certain embodiments, the pharmaceutical composition further comprises a surfactant. The surfactant may be selected from group consisting of polysorbate 80, pemulen TR-1, Arlacel 165, castor oil, hydrogenated castor oil, propylene glycol monolaurate, caprylic triglycerides, capric triglycerides, glycerol stearate, PEG stearate, and any combination thereof.

In certain embodiments, the pharmaceutical composition further comprises a glidant. The glidant may be selected from a group consisting of silica, cyclomethicone, magnesium stearate, and any combination thereof.

In certain embodiments, the pharmaceutical composition further comprises a pharmaceutically acceptable buffer. The pharmaceutically acceptable buffer may be selected from phosphoric acid, citric acid, salts thereof, or any combination thereof. In certain embodiments, the pharmaceutical composition comprises from about 0.01% (w/w) to about 5% (w/w) of a pharmaceutically acceptable buffer.

In certain embodiments, the pharmaceutical composition may further comprise excipients selected from group consisting of: petrolatum, diethyl sebacate, coconut oil, stearyl alcohol, and any combination thereof.

Also provided are processes for preparing the pharmaceutical compositions of the invention for topical application, the process comprising: mixing a solvent, an activin receptor-like kinase-5 (ALK-5) kinase inhibitor, a permeation enhancer, an antioxidant, a thickening agent, and optionally a preservative to thereby produce a pharmaceutical composition for topical application.

Further provided are methods of treatment of diseases or disorders of the skin or affecting the skin by topical administration of a pharmaceutical composition of the invention to a patient, the pharmaceutical composition comprising: an activin receptor-like kinase-5 (ALK-5) kinase inhibitor; a permeation enhancer; a solvent; an antioxidant; a thickening agent; and optionally a preservative. Preferably, the patient is a human. In some embodiments, the patient is a veterinary animal. Also provided are uses of the pharmaceutical compositions of the invention for treatment of diseases or disorders of the skin or affecting the skin, as well as pharmaceutical compositions of the invention for use in methods of treatment of diseases or disorders of the skin, or affecting the skin.

In preferred embodiments, the pharmaceutical composition is designed for treatment of topical conditions. Diseases or disorders that can be treated by the pharmaceutical compositions of the invention include, but are not limited to, scars, hypertrophic scars, keloid scars, keloid morphea, skin fibrosis, scleroderma, skin cancer, basal cell carcinoma, squamous cell carcinoma, melanoma, cutaneous neurofibromas, cutaneous lupus erythematosus, discoid lupus erythematosus, hidradenitis suppurativa, dupuytrene's contracture and Peyronie's disease. In one embodiment, the disease or disorder is dupuytrene's contracture and the pharmaceutical composition is applied to the skin of the hand. In another embodiment, the disease or disorder is Peyronie's disease and the pharmaceutical composition is applied to the skin of the penis. In still another embodiment, the disease or disorder is a fat remodeling disorder. The fat remodeling disorder can be, for example, one or more of submental fat, lipomas, xanthelasma, paradoxical adipose hyperplasia, piezogenic pedal papules, and HIV associated lipohypertrophy. In another embodiment, the disease or disorder is alopecia. The alopecia can be, for example, one or more of patterned alopecia, central centrifugal cicatricial alopecia (CCCA) and alopecia areata.

Preferably, the ALK-5 kinase inhibitor is a highly potent small molecule inhibitor of ALK-5 kinase. Preferably, the inhibitor will block TGF-β signaling leading to excessive fibrosis. Preferably, the pharmaceutical composition comprises a topical locally-acting drug. Systemic toxicity of ALK-5 kinase inhibitors has previously hindered development of therapies involving these inhibitors. The pharmaceutical compositions of the invention overcome this problem by delivering high levels of the drug to the skin as compared to the systemic circulation.

In certain embodiments, the pharmaceutical compositions of the invention may be used for the treatment of fibrotic indications. This is an area which presents long-felt but unmet needs for the patients suffering from these diseases. In certain embodiments, the pharmaceutical composition of the invention is used for the treatment of scleroderma. In certain embodiments, the pharmaceutical compositions of the invention are beneficial for treatment of skin fibrosis. In certain embodiments, the pharmaceutical compositions of the invention are used for functional improvement of impaired hand and/or mouth function. In certain embodiments, the pharmaceutical compositions of the invention are used for treatment of keloid scars.

The pharmaceutical compositions of the invention may also be used for treatment of skin cancers. In certain embodiments, the pharmaceutical compositions of the invention are used for the treatment of skin squamous cell cancer.

The invention further provides processes for preparing the pharmaceutical compositions comprising ALK-5 kinase inhibitors. In certain embodiments, the process comprises mixing a solvent, an ALK-5 kinase inhibitor, a permeation enhancer, optionally a preservative, an antioxidant, and a thickening agent to thereby produce a pharmaceutical composition for topical application. In certain embodiments, the solvent comprises an aqueous solvent and a non-aqueous solvent. In certain embodiments, the mixing steps involve dissolving hydrophobic excipients in the non-aqueous solvent to form a non-aqueous solution. In certain embodiments, the aqueous solvent is mixed with the non-aqueous solution to form a mixture. In certain embodiments, the process comprises addition of the thickening agent to the mixture.

In an aspect, the invention further provides a preparation of pharmaceutical compositions of the invention. In certain embodiments, the process comprises: mixing a solvent, an ALK-5 kinase inhibitor, a permeation enhancer, an antioxidant, and a thickening agent to thereby produce a pharmaceutical composition for topical application. In certain embodiments, the solvent comprises an aqueous solvent and a non-aqueous solvent. In certain embodiments, the mixing step involves dissolving hydrophobic excipients in the non-aqueous solvent to form a non-aqueous solution in a specific order. In one embodiment, the aqueous solvent is mixed the non-aqueous solution to form a mixture. In one embodiment, the process further comprises addition of the thickening agent to the mixture in a specific order.

### BRIEF DESCRIPTION OF THE DRAWINGS

FIG. 1 provides an overview of the complexity and the relevant factors in development of topical pharmaceutical compositions.

FIG. 2 provides an overview of various types of topical formulations applied to the skin that need to be balanced with the disease being treated in developing the pharmaceutical compositions.

FIG. 3A provides data regarding the delivered dose of various tested pharmaceutical compositions.

FIG. 3B discloses data regarding the deposition of Compound 1 in the dermis and epidermis 24 hours after application of various formulations.

FIG. 4 provides data regarding the concentration of Compound 1 in the skin and the plasma after application of pharmaceutical compositions of the invention.

FIG. 5 provides the Draize erythema score of various tested formulations.

FIG. 6 provides the pharmacokinetic profile for various compositions comprising Compound 1.

FIG. 7 provides a heatmap of gene expression in minipig skin following treatment with Compound 1.

### DETAILED DESCRIPTION OF THE INVENTION

The invention is directed to pharmaceutical compositions of ALK-5 kinase inhibitors. In particular, the invention provides topical formulations of ALK-5 kinase inhibitors for application on skin for treatment of skin diseases and disorders. In particular, the pharmaceutical compositions may be used for treatment of diseases and disorders such as scleroderma, keloid scars, and skin cancers, in particular diseases and disorders that do not have any currently available therapies.

### Therapeutic Uses

Therapeutic uses of the pharmaceutical compositions of ALK-5 kinase inhibitors include, but are not limited to, the following.

### Scleroderma

There are two major classifications of scleroderma: systemic sclerosis and localized scleroderma (often referred to as morphea). Localized scleroderma is a condition where fibrosis affects the skin (with occasional deeper penetration below the dermis). In contrast, in systemic sclerosis, fibrosis extends well beyond the skin and involves fibrosis of multiple internal organs. Skin fibrosis in localized scleroderma and systemic sclerosis has different clinical manifestations but histologically can appear identical (Walker et al 2017).

Systemic sclerosis is a chronic autoimmune disease characterized by vasculopathy, diffuse fibrosis of the skin and various internal organs (Denton et al 2017). There are two main types of systemic sclerosis differentiated by the extent of proximal extremity involvement:
1. Limited systemic sclerosis (formerly called CREST [Calcinosis, Raynaud's phenomenon, Esophageal dysmotility, Sclerodactyly, and Telangiectasias] syndrome), with skin disease confined to the fingers, hands and forearms distal to the elbows, as well as the knees and face; and
2. Diffuse systemic sclerosis also involves the fingers, hand and forearms distal to the elbows, but the proximal extremities and trunk also are involved.

Patients with diffuse disease have a challenging prognosis with pulmonary fibrosis, pulmonary hypertension, severe GI involvement, and heart disease being the main causes of death. However, cutaneous fibrosis in these patients has been noted as having the greatest impact on quality of life, particularly in the initial phases of the disease. The development of thickened skin leads to joint contracture, Raynaud's phenomenon, and ischemic digital ulceration. Hand function impairment, termed sclerodactyly, is observed in >90% of systemic sclerosis patients (Young et al 2016).

Localized scleroderma, in contrast, is not life-threatening, but quality of life is adversely affected because of compromised appearance and joint contracture effecting movement. In children, if fibrosis is severe, significant limb and bone disfigurement can also occur.

### Importance of TGF-β/ALK-5 in Cutaneous Scleroderma

TGF-β is well established to be a key growth factor regulating the activation status of dermal fibroblasts in systemic sclerosis (Ayers 2018, Lomeli-Nieto 2022 Rice et al 2015). TGF-β promotes inflammation by recruiting and activating leukocytes and inducing various pro-inflammatory cytokines in the early stage of systemic sclerosis (Luong 2018). TGF-β inhibition is widely considered a promising target in systemic sclerosis, however, the safety of systemic inhibition remains a major concern (Budi 2021).

The most convincing data supporting a role for TGF-β in cutaneous scleroderma comes from the therapeutic efficacy of a TGF-β neutralizing antibody, fresolimumab, which targets all three TGF-β isoforms (Rice et al., 2015). In this study, systemic sclerosis patients (N=15) with disease duration of <2 years were treated either twice with a low dose (1 mg/kg), 4 weeks apart, or once with a high dose (5 mg/kg), and then monitored for up to 24 weeks. The results showed that clinical skin disease (fibrosis) was dramatically reversed, as assessed using the modified Rodnan skin score (MRSS), with very significant effects (p=0.0002) observed within just 3 weeks after dosing with fresolimumab. In addition, pro-fibrotic TGF-β gene expression profiles in the skin were decreased in parallel with the resolution of skin fibrosis. Together, these data provide clear evidence that TGF-β is involved in the development of skin fibrosis and that blockade of all the three TGF-β isoforms by an ALK-5 kinase inhibitor is a promising therapeutic strategy for treating cutaneous scleroderma in systemic sclerosis. Further use of fresolimumab to treat systemic sclerosis was terminated after this study, due to significant systemic side effects, notably bleeding. Two patients developed clinically significant gastrointestinal bleeding from the gastric antral vascular ectasia and both required transfusion. Gingival bleeding and/or epistaxis were described in three patients, and subconjunctival hemorrhages in 2 patients.

A topical ALK-5 kinase inhibitor, which achieves high levels in the dermis but with low systemic exposure, has the potential to reduce skin fibrosis in patients with systemic sclerosis, as well as in localized scleroderma, but without the side effects of systemic TGF-β inhibition.

### Keloid Scarring

Skin scars can be divided into 3 broad types: fine line scars, hypertrophic scars and keloid scars. Fine line scars and hypertrophic scars, while of psychological concern for many subjects, are generally a cosmetic issue only. Fine line scars are self-healing by 6 months, with a peak of severity at 3 months. Hypertrophic scars are most typically linear, raised, but restricted to the line of injury. They arise 4-8 weeks post injury, peak in severity within 6 months, and then can either begin to flatten over the next several years or remain raised for a decade or more. In contrast, keloid scars, while never malignant, grow beyond the site of the initial injury. They can begin to grow as early as one-month post-surgery or injury, grow for multiple years, and do not spontaneously resolve. Keloid scars can also become very large and can be extremely disfiguring. Keloid scars can be pruritic and may be a significant source of pain. Hyperpigmentation is common.

### Importance of TGF-β/ALK-5 in Keloid Formation

Aberrant scar formation, including keloid scars, are associated with disorganized wound healing and chronic inflammation that has its basis in sustained TGF-β1/SMAD signaling (Zhang et al., 2020). TGF-β1 has been reported to be significantly elevated in both keloid derived fibroblasts (Wang et al., 2021), keloid derived keratinocytes (Hahn et al., 2013) and in keloid tissue (El-Aleem et al., 2017). Ke et al. (2021) showed downregulation of SMAD3 decreases both collagen gene expression and ECM deposition by keloid fibroblasts. Accordingly, a small molecule inhibitor of ALK-5 demonstrated efficacy in fibroproliferative dermal fibroblasts (Wang et al 2021), enhanced rates of in vitro wound closure, suppressed fibrotic genes and increased expression of anti-fibrotic genes (Peterson et al., 2022).

A topical ALK-5 kinase inhibitor, which achieves high levels in the dermis but with low systemic exposure, has the potential to prevent aberrant scar formation, including keloid scars, without the side effects of systemic TGF-β inhibition.

### Non-Melanoma Skin Cancer (NMSC)

The two most common cancers world-wide, both occur in the skin and arise from transformed keratinocytes: namely basal cell carcinoma (BCC) and cutaneous squamous cell carcinoma (cSCC) (world wide web .cancer.org/cancer/types/basal-and-squamous-cell-skin-cancer). The incidence of BCC is higher than that of cSCC, but the risk of metastasis, while still low, is much greater for cSCC than BCC, and thus the overall mortality is higher. Taken together, BCC and low risk cSCC provide very large patients populations in need of surgery-sparing treatment options, while more advanced and/or recurrent cSCC represents a high medical need population in need of new life-saving therapies.

### Cutaneous Squamous Cell Carcinoma

The majority of localized, low-risk cases of cSCC are managed with destructive or surgical techniques, such as curettage and electrodessication, or standard wide local excision with margins of 4-6 mm, providing cure rates of over 90% (Wysong, 2023). However, despite the effectiveness of these treatments, a well-tolerated and easy to use topical treatment with a similarly high cure rate would provide an attractive surgery-sparing option for many patients with low risk cSCC.

Although about 95% of patients with cSCC have relatively small, localized disease that is generally excised or treated with one of the various ablative techniques, the remaining ˜5% percent with highly recurrent, locally advanced or metastatic disease. Pharmaceutical options for these patients had been very limited until the recent introduction of two checkpoint inhibitors, cemiplimab and pembrolizumab; however, there is a clear need for further improvement. Many advanced cSCC patients are not responsive to anti-Programmed Cell Death Protein 1 (anti-PD-1) therapy, and significant number of patients also develop resistance to such therapy. Thus, another clear clinical opportunity for topical ALK-5 kinase inhibitors in cSCC is in particular locally advanced disease in patients who have failed anti-PD-1.

Immunosuppressed patients represent another population where new therapy is needed. In the registrational trials with cemiplimab and pembrolizumab, patients having a medical condition requiring systemic immunosuppression, such as organ transplants, were excluded in these trails due to significant risk of organ rejection on such therapy. Topical ALK-5 kinase inhibitors should pose a negligible risk of organ rejection to transplant recipients due to a lack of systemic activity but offer the potential to re-engage an immune response towards the cSCC lesions where it is applied. Thus, a potentially attractive therapeutic concept would be to drive rapid complete responses in solid organ transplant patients by combining topical ALK-5 kinase inhibitors with systemic anti-PD-1 therapy, while temporarily managing the intensity of the immunosuppressive therapy

Importance of TGF-β/ALK-5 in cSCC

In 2018, there were two seminal publications in Nature, documenting the key role of TGF-β in the effectiveness of anti PD-1 immune therapy (Tauriello et al 2018; Mariathason et al 2018). First, TGF-β is overexpressed in many tumors. TGF-β is a potent immunosuppressive agent, which blocks an immune response. Secondly, this local overexpression creates a fibrotic microenvironment, which minimizes both immune cell access and drug access to the tumor. Hence the combination of an ALK-5 kinase inhibitor with an anti PD-1 therapy became a clear objective, if a way could be found to achieve that safely. In the context of topical delivery of ALK-5 kinase inhibitors to cSCC tumors then, the primary mechanistic goal is not to directly target TGF-β pathway signaling in the cancer cells per se (though that may provide additional benefit), but to target the tumor microenvironment and thereby promote an effective antitumor immune response, by decreasing both the immunosuppressive activity of TGF-β and local fibrosis, to enable greater immune cell access.

In supporting animal data, the combination of anti-PD-1 and TGF-β antibodies dramatically improved efficacy relative to either single agent in a mouse model of cSCC (Dodagatta-Marri et al., 2019). It is also worth noting, that single agent anti-TGF-0 was more efficacious than anti-PD-1 therapy alone in this mouse model. Furthermore, the animals in which the tumors were completely rejected, remained completely resistant to a subsequent re-challenge with the same cSCC cell line even 18 months later, demonstrating immune memory.

Clinical data from early trials of STP705 in patients with cSCC in situ provide additional support for the importance of TGF-β in cSCC. STP-705 (Sirnaomics) is an injectable nanoparticle that contains siRNAs targeting both TGFβ-1 and COX2. In a small single arm dose-ranging pilot study, STP705 demonstrated high rates of complete histological clearance at doses of 30 to 120 mg injected weekly for up to 6 weeks (Nestor et al., 2022).

### Basal Cell Carcinoma (BCC)

Like cSCC, BCC is associated with mutational damage due to sun exposure and has a high mutational burden. Unlike cSCC, however, only a very small percentage (<<1%) of BCC patients progress to locally advanced or metastatic disease. Sirnaomics has recently reported that STP-705, an injectable nanoparticle that contains siRNAs targeting both TGFβ-1 and COX2, has shown histological clearance of superficial BCC in a small Phase 1 cohort. These results, together with limited literature indicating a role of TGF-β in the BCCs (Fan et al., 2010; Yao et al., 2020), provide a rationale for the use of topical ALK-5 kinase inhibitors in BCC.

### Cutaneous Lupus (Especially Discoid Lupus Erythematosus)

Cutaneous lupus erythematosus (CLE) is characterized by skin symptoms that fall under the spectrum of lupus erythematosus conditions (Kuhn, 2014). CLE may occur with or without the involvement of systemic lupus erythematosus (SLE). Discoid lupus erythematosus (DLE) is a chronic form of cutaneous lupus, which can cause scarring. DLE most commonly occurs on the head and is characterized by well-defined, inflammatory plaques that evolve into atrophic, disfiguring scars. Subacute cutaneous lupus erythematosus (SCLE) typically presents with inflammatory, scaly papules or anular plaques on the neck, upper trunk and arms. Both SCLE and DLE can occur in association with systemic lupus erythematosus (SLE), although co-occurrence of SCLE with SLE is more frequent. SCLE is commonly ANA and Ro positive whereas DLE is often ANA negative. Drug-induced SCLE presents with cutaneous and serologic findings similar to idiopathic disease.

No medications have been approved for CLE in over 50 years (Guo, 2021). While some treatments are effective for CLE, some patients remain refractory or cannot tolerate current therapies. Development of novel therapies is stymied by a lack of harmonization on valid, reliable and clinically meaningful disease severity measures, heterogeneity in outcome reporting and a lack of regulatory approval guidelines (Galoppini, 2023). From a patient perspective, the disfigurement of CLE and DLE can impact quality of life. Early effective treatment may lead to total clearing of the skin lesions, but failure of treatment results in permanent scarring. The depressed scars, hair loss, and pigmentary changes are often extremely disfiguring, particularly in darker-skinned people. Permanent scarring is the rule if treatment is delayed or inadequate. Great emotional distress, social isolation, and difficulty obtaining work are problems frequently suffered by these patients. Hazard ratios for depression in patients with cutaneous SE are estimated at 2.07 (95% CI 1.55-2.75) with up to 35% of patients meeting criteria for depression/anxiety. Involvement of the skin of the fingers and toes may markedly impair hand function and limit walking (Vasquez, 2013). In addition, people with DLE are at risk of developing squamous cell carcinoma in the scarred areas (Fernandes, 2015).

### Importance of TGF-β/ALK-5 in DLE

Although the molecular mechanisms leading to fibrosis in DLE are not well understood, microarray data show a strong involvement in epithelial-mesenchymal transition (EMT) pathways. In particular, the TGF-β signaling pathway is overexpressed in DLE skin samples and these effects are linked with EMT (Sole, 2016). Relative to non-lesional biopsies in the same subjects, skin biopsies from DLE show increased gene expression of TGF-β1 its receptor and intracellular signaling components (SMAD3) and fibrotic target genes (MMP9, MMP1 and SERPINE1). These findings were further supported by immuno-histochemical studies and ex vivo studies in fibroblasts suggesting that the TGF-β pathway is strongly involved. For example, primary isolated fibroblasts from DLE and SCLE showed increased production of fibrotic markers following TGF-β, but stimulation by other cytokines and growth factors did not induce these changes (Sole 2016).

ALK-5 phosphorylates the receptor activated SMADs, which are downstream mediators of TGF-β signaling, which in turn drives transcription of collagen genes and matrix deposition. Small molecule inhibitor of ALK-5 kinase would be expected to help resolve and/or reverse scarring processes in SCLE and DLE. A topical locally-acting ALK-5 kinase inhibitor would be expected to achieve this therapeutic benefit in the absence of systemic side effects.

### Peyronie's Disease

Peyronie's disease (PD) is an acquired connective tissue disorder that results in the development of penile plaques in the thick elastic membrane called the tunica albuginea of the corpora cavernosa. The tunica albuginea is bilaminar with an inner circular and outer longitudinal layer. During normal penile erection, nitric oxide is released increasing penile blood flow and relaxation of cavernosal smooth muscle. In turn, hydrostatic pressure increases leading to compression and occlusion of small venules that perforate the tunica aluginea. In PD, normal collagen architecture is lost with disordered deposition of collagen I and III, fibrin and elastic fibers similar to the formation of hypertrophic scars (Taylor, 2007). Deposition of scar tissue leads to decreased stretch and elasticity leading to penile curvature. The etiology is thought to be due to disordered wound healing caused by trauma primarily during sexual intercourse. The delamination of the tunica albuginea leads to bleeding and clot formation and fibrin degradation stimulates growth factors like TGF-β 1, fibrin and plasminogen activator inhibitor-1 leading to scarring of the inner lamina, plaque formation and penile curvature.

Patients (usually Caucasian 40-60 yo) present with penile curvature or deformity during erection (60-94%), penile pain (20-70%, usually only during erections) and erectile dysfunction (ED) with a palpable plaque (Ostrowski, 2016). During the acute phase there is penile pain, a soft plaque and curvature that may increase, not change or improve. The chronic phase (>12 months) is characterized by stability in penile curvature, resolution of pain and a hardening and calcification of the plaque. PD negatively impacts body image, mood, sexual relationships and quality of life. Imposed restrictions on sexual intercourse leads to feelings of shame, stigmatization, and social isolation. Approximately 80% of men with PD report emotional difficulties including moderate (26%) to severe (21%) clinical depression (Terrier, 2016). Female partners report decreased sexual function and relationship dissatisfaction.

Non-surgical management is limited to the acute phase and seeks to resolve penile pain, inhibit further curvature, improve erectile function and restore penile length. Treatment options include extracorporeal shockwave treatment (ESWT; mechanical stimulation of cells, stimulation of NO and VEGF), penile traction therapy (PTT; improve penile length by stretch), vacuum pump device (VP; mechanical straightening of curvature) and Collagenase Clostridium histolyticum (CCH; enzymatically breakdown collagen) injections. A recent Cochrane review considered 14 trials in 1810 men with Peyronie's disease that did not involve a surgical procedure (Rosenberg, 2023). Key outcomes for Peyronie's include a patient's self-reported ability to have intercourse, improved quality of life, and side effects. They also considered penile curvature. Overall, injected verapamil, ESWT and penile traction therapy were met for low or very low certainty of evidence. The certainty of evidence for collagenase injections for long term outcomes was considered moderate (Rosenberg, 2023). Surgical management may be considered for patients who have penile deformity compromising sexual function and whose PD has persisted for more than 12 months, and is refractory to medical therapy. Surgical intervention remains the mainstay of treatment for PD (Chung, 2020), but is not without risk. Potential complications include penile shortening, sensation change, recurrence of curvature and erectile dysfunction.

### Importance of TGF-β/ALK-5 in Peyronie's Disease

Convergent lines of evidence support that TGF-β inhibition, via ALK-5, should have therapeutic value in PD. First, TGF-β is a central regulator of tissue inflammation, repair, remodeling and fibrosis. As an early mediator produced in response to cell injury, TGF-β stimulates myofibroblasts proliferation and collagen for early ECM deposition and induces chemotaxis of cells involved in inflammation and fibrosis including neutrophils, macrophages, monocytes and lymphocytes. TGF-β also alters production of key mediators in the inflammatory and wound healing response and inhibits mediators of collagenolysis (MMP-1, MMP-8 and MMP-13) and fibrinolysis (PAI-1). In PD, MMPs are over-inhibited, leading to excess collagen and ECM leading to plaque formation.

Peyronie's plaques have been shown to have increased TGF-β1 without increases in TGF-β2 or TGF-β3 suggesting that TGF-β1 is critical (Taylor, 2007). Genetic variations in the coding region of TGF-β1 have been reported in patients with PD (Hauck, 2003). TGF-β1 has also been used to induce PD in a rat (Chung, 2011) and rabbit model (Gundogdu, 2023) model further solidifying its role in the disease.

When TGF-β binds to ALK-5 serine/threonine residues in ALK-5 are phosphorylated and the activated ALK-5 subsequently phosphorylates the downstream signaling molecules Smad2/Smad3. In turn, Smad2/3 heterotrimerizes with Smad4, which translocates into the nucleus and regulates transcription of TGF-β responsive genes. Expression and activity of the SMAD transcription factors of the TGF-β pathway are known to be increased in fibroblasts from PD patients (Haag, 2007). Furthermore, administration of the ALK-5 inhibitor, IN-1130, improved curvature and fibrosis in rat PD models (Ryu, 2007). IN-1130 reduced infiltration of inflammatory cells, attenuated transnuclear expression of pSMAD2 and pSMAD3, suppressed collagen accumulation and restored elastin fibers by inhibiting macrophage recruitment. In PD-derived fibroblasts IN-1130 significantly reduced TGF-β1-induced production of PAI-1, fibronectin, collagen 1 and collagen 4. Another small molecule inhibitor of ALK-5, SKI2162, was also shown to block TGF-β1 induced signaling (Smad2/Smad3) and inhibit extracellular matrix production in fibroblasts derived from human PD plaque (Piao, 2010). Vactosertib, which was developed as a highly potent, selective and orally bioavailable ALK-5 inhibitor for use as an anti-fibrotic and cancer immunotherapeutic agent was tested in the PD rat model. As with the other ALK-5 inhibitors, Vactosertib administration induced regression of fibrotic plaques through reduced infiltration of inflammatory cells (Song, 2020). Vactosertib also reduced expression of pSMAD2 which recovered erectile function. Additionally, the ALK-5 inhibitor counteracted TGF-β1 induced ECM production and hydroxyproline content in fibroblasts by impeding phosphorylation and nuclear translocation of SMAD2/3 and fibroblast-to-myofibroblast transdifferentiation.

Given these findings, the ALK-5 kinase inhibitors represent a non-surgical approach that would be expected to improve PD through the following mechanisms: (1) reduce inflammatory cell infiltration to promote regression of plaques, (2) suppress transnuclear expression and phosphorylation of Smad2/3, (3) inhibit myofribroblastic differentiation induced by TGF-β 1, and (4) decrease production of collagen and ECM components induced by TGF-β 1.

### Dupuytrene's Contracture

Dupuytrene's contracture (DC) is a benign hyper-proliferative condition affecting the palmar fascia of the hand and is the most common organ-specific fibrotic disease (Broekstra, 2023). As fibroblasts differentiate to myofibroblasts with contractile properties there is disorganized, poorly cross-linked and excessive collagen deposition, which may cause progressive contractures of the fingers. It is most prevalent in the ulnar rays of the hands, but can involve all the fingers. Contractures lead to problems with hand function and interfere with activities of daily living. The gold-standard treatment for Dupuytrene's contracture has involved surgery, ranging from percutaneous release to dermatofasciectomy. While these procedures have a high short-term success rate, there is a high rate of recurrent contracture. Additionally, the procedures can be associated with additional comorbidities such as wound-healing complications and neurovascular injury. Minimally invasive options include percutaneous needle aponeurotomy (PNA) and collagenase clostridium histolyticum (CCH) injections (Xiaflex®, the only drug approved for DC; Soreide, 2018).

Current treatments of DC are limited to late-stage disease when patients have developed flexion contractures (a fixed flexion deformity at the MCP joint of 30 degrees or PIP joint of 15 degrees). As noted above, DC is often treated using a variety of surgical techniques. Insufficient evidence is available to show the relative superiority of different surgical procedures (needle fasciotomy vs. fasciotomy, or interposition firebreak skin grafting vs. z-plasty closure of fasciectomy; Rodrigues, 2015). Reported side effects from these procedures may include altered feeling in the fingers or reduced ability to make a full fist. Rare complications may include injury to the tendons that pull the fingers into the palm. The only approved pharmacotherapy for treating DC is clostridial collagenase (CCH) which preferentially degrades collagen type I and III in DC contractures. A recent systematic review found that treatment success was high for CCH injection with rates of 76% for MCPJs and 31% of PIPJs (Sandler, 2021). Identified success factors for CCH include when the contracture is limited to a single MCPJ cord and/or joints with less severe initial contractures. Adverse events are typically minor with CCH and are self-resolving and limited to the injection site. An advantage of CCH is that it has lower complication rates than surgery, however, ˜25% of successfully treated joints experience recurrence. CCH therapy is also considerably more costly (up to 43 times) than the least expensive surgical approach of PNF (Stromberg, 2017). Additionally, CCH is not readily available outside of the USA. Corticosteroids (injectable triamcinolone have also been used in patients with DC. Mechanistically, it is believed that corticosteroids decrease rates of cell proliferation in DC nodule and cells and may also inhibit TGF-B1 expression and fibroblast apoptosis. Unfortunately, there are a lack of long-term trials available to support their use in DC (Lambi, 2023). TNF has been identified as a therapeutic target and, in a Phase 2a trial, intranodular adalimumab downregulated the phenotype of myofibroblasts (Nanchahal, 2018). In a subsequent Phase 2b trial adalimumab decreased nodule harness and size by ultrasound (Nanchahal, 2022). Overall, the highest unmet need may be for the earliest stage of the disease as surgery and CCH injections are only available for later stages of DC.

### Importance of TGF-β/ALK-5 in Dupuytrene's Contracture.

Multiple downstream effectors of the TGF-β pathway are dysregulated in DC including mRNA and protein expression levels of fibrous collagens (I and IIII), smooth muscle actin, fibronectin, MMPs and integrins (Ratajczak-Wielgomas, 2012). It is clear that the activation and trans-differentiation of DC fibroblasts to a myofibroblasts phenotype is mainly controlled by TGF-β and that in patient derived myofibroblasts cultures, overactive TGF-β signaling leads to contraction and proliferation (Krause, 2011). All three isoforms of TGF-β have been identified in DC disease nodules, palmar fascia and cord tissue (Berndt, 1995; Baird, 1993). Additionally, TGF-β signaling is upregulated in DC and is expressed in fibroblasts and myofibroblasts at all three histological stages of disease progression. TGF-β1 addition to cultures of DC fibroblasts upregulates α-SMA expression and induces differentiation of a quiescent fibroblast to a contracting myofibroblasts whether the cells were obtained from DC affected or unaffected tissues. The addition of TGF-β in culture models leads to increased contracture of DC fibroblasts (Tse, 2004) while blockage of TGF-β signaling can dose-dependently decrease contractility, α-SMA and Col1 gene expression and protein (Verjee, 2013).

Inhibiting uncontrolled fibrotic mechanisms by directly targeting overactive TGF-β signaling via the ALK-5 receptor kinase is a rational approach. SB-431542 (which targets ALK-5, as well as ALK-4 and ALK-7) suppressed TGF-β-induced expression of ACTA2, Col1a1 and Col3a1 in a novel 3D culture model of DC-specimens (Krause, 2011). To prevent potential off-target effects interfering with broad TGF-β signaling, an antisense oligonucleotide-mediated exon skipping technology was also used to disrupt the protein function of ALK-5 in 3D cultured DC patient-derived resection specimens. Injecting the antisense oligonucleotides directly into the tissue specimen inhibited both the SMAD-dependent and non-dependent TGF-β signaling pathways, and decreased collagen protein expression and degradation/reorganization of collagen structures (Karkampouna, 2014).

Small molecule inhibitors of ALK-5 kinase would be expected to have similar effects to downregulate TGF-β signaling and downstream pathways as these other approaches.

Cutaneous Neurofibromas (cNF)

Neurofibromatosis type I (NF1; von Recklinghausen's disease) is a neuro-cutaneous disorder characterized by a mutation or deletion of tumor suppressor gene, NF1 (neurofibromin; Anderson, 2015). NF1 is abundantly expressed in neurons, oligodendrocytes and Schwann cells and normally inhibits the proto-oncogene RAS. The loss of expression leads to the development of multiple tumors on nerves throughout the body. NFs can be classified according to their anatomical location: (1) Plexiform NFs involve nerve plexus below the dermis, (2) Intra-neural growths within the peripheral nerves, (3) Subcutaneous NFs along the peripheral nerves beneath the skin, and (4) Cutaneous or dermal NFs (cNFs) grow from small nerves present in and/or just under the skin. NFs look like well-defined cutaneous lesions. cNFs typically begin around the time of puberty, increase with age and undergo periods of accelerated growth in puberty and pregnancy. They are the most common tumor manifestation in adults as they affect 99% of NF1 patients (Cannon, 2018). cNFs are treated with surgery and sometimes with CO₂ laser therapy or electrodessication.

NF1 disease burden also includes susceptibility to aqueductal stenosis, pheochromocytoma, learning/intellectual disabilities, attention deficit, scoliosis, vasculopathy and other types of tumors and malignancies. Dermal NFs first appear around the time of puberty, and they typically increase in number with age. While these tumors are benign and do not transform into malignant cancers, they are associated with significant cosmetic impact or cause irritation due to rubbing or clothing irritation. Surveys of cNF patients suggest that poor aesthetics is the biggest burden but secondary aspects of pain and itching symptoms are considered the most bothersome (Guiraud, 2019). Patients would consider a future treatment to be moderately effective if it could clear 30% of the cNFs. Due to the abundance of cNFs in a patient removing all lesions is usually not feasible. A scar will result at each site and tumors are prone to regenerate from any remaining cells. Non-surgical treatments and clinical trials have been hampered by difficulties in quantifying/measuring cNFs and lack of detailed information on the natural history of the lesions. There is no treatment approved to cure cNF and treatment is limited to surgical excision or destruction via laser or electrodessication. Systemic and topical treatments for cutaneous neurofibromas are summarized in Poplausky (2023). However, no successful results have yet been reported for topically applied monotherapies in cNF (Allaway, 2018).

### Importance of TGF-β/ALK-5 in cNF

Although inactivation of the NF1 tumor suppression disease leads to increased Ras signaling, targeting pathways of Ras signaling in Schwann cells have not been effective at regressing cNF (Brosseau, 2021). The NF microenvironment is comprised of fibroblasts, pericytes, immune cells (such as macrophages, mast cells), and blood vessels mingled in a thick collagenous matrix. While it is unlikely that ALK-5 inhibition would directly impact the Ras pathways, the pro-inflammatory and pro-fibrotic tumor microenvironment (TME) modulates neuro-fibroma development and is under control of TGF-β1 signaling (Jiang, 2023). Single cell sequencing studies reveal a regulatory network whereby immune cells including macrophages and T cells produce TGF-β1 to induce Schwann cells to produce and deposit BM proteins for ECM remodeling. Studies of the ECM in pNF reveal a regulatory network in NFs whereby NF1 loss and TGF-β1 upregulation lead to basement membrane protein deposition by Schwann cell (Jiang, 2023). ALK-5 kinase inhibitors have been shown to regulate multiple components of the ECM in wound healing models, so it would be expected to have beneficial effects in cNF.

### Keloid Morphea

Keloidal morphea (KM) is a rare variant of localized scleroderma, a chronic connective tissue disease of unknown etiology. It is often confused with keloid or scar formation and presents with relatively few, large, firm, brown or violaceous plaques with pseudopod-like extension resembling a keloid (Rencic, 2003). The plaques eventually resolve but leave permanent dermal or soft tissue atrophy and pigmentary changes. As of 2020, less than 50 cases of keloidal morphea have been described in the literature (Dadkhahfar et al., 2020). The majority of patients are treated with topical or intra-lesional medications (corticosteroids, calcineurin inhibitors, calcipotriene), phototherapy (UVA, UVB and psoralen+UVA), or systemic therapies (methotrexate, mycophenolic acid, IVIg, JAK inhibitors, and IL-4 antagonists; Goldman, 2022).

### Importance of TGF-β/ALK-5 Inhibition in Keloid Morphea

The subtype of keloid morphea is rare and its pathology not well described, but a handful of gene expression studies from keloid morphea have been conducted. In keloid morphea, Yamamoto (2005) reported, in a small case series, increased connective tissue growth factor (CTGF; CCN2) expression at the mRNA and protein levels in fibroblasts in the lesional skin of keloid morphea in 3 patients with concurrent keloid morphea and diffuse SSc. Importantly, ALK-5 kinase inhibitors also reduces CTGF mRNA suggesting potential in rare fibrotic skin diseases like keloid morphea. Clark (2023) compared skin gene expression from the keloidal lesions with that from skin of healthy controls and found that multiple fibrotic genes under the control of TGFβ-1 were upregulated. They noted that the gene signature in keloid morphea appeared an exaggeration of the gene signature typical seen in systemic sclerosis. Thus, given that ALK-5 kinase inhibitors have shown efficacy in animal models of dermal fibrosis by inhibiting the downstream effects of TGFβ-1, one would expect therapeutic potential in this indication.

### Hidradenitis Suppurativa

Hidradenitis suppurativa (HS; also known as ‘acne inversa’ or ‘Verneuil's disease’) is a distressing chronic inflammatory skin condition with recurrent painful boils in skin creases of flexural sites such as the armpits, groin, and genitals. The lesions are recurrent tender nodules or subcutaneous abscesses. These can lead to sinus tracts that discharge purulent fluid. The age of onset of HS is typically early adulthood and HS significantly impacts quality-of-life due to the pain, scarring and effects on self-esteem.

HS is currently considered a disease of the pilosebaceous unit associated with an immune imbalance in a genetically predisposed individual (Diaz, 2022). The primary defect in HS pathophysiology involves occlusion and subsequent inflammation of the hair follicle. Innate and adaptive immune dysregulation initiate the development of clinical HS. Bacterial infection and colonization are secondary pathogenic factors that can worsen the disease. Follicular occlusion leads to dilatation followed by rupture of the follicular contents, including keratin and bacteria, spilling into the surrounding dermis. In turn, this induces a vigorous chemotactic response from neutrophils and lymphocytes and the inflammatory cellular infiltrate causes abscess formation, leading to the destruction of the pilosebaceous unit and adjacent structures (Napolitano, 2017). Other factors that may contribute to HS include altered expression of antimicrobial peptides, abnormal secretion of apocrine glands, abnormal invaginations of the epidermis leading to sinus tract formation, and deficient numbers of sebaceous glands.

Topical antibiotics (most notably clindamycin) are first-line therapy for superficial lesions but are ineffective with deeper lesions and more advanced disease. Systemic antibiotic combinations are used for more severe disease. Adalimumab is the only approved medication for moderate-to-severe HS to date (Tsai, 2023). There are still a number of patients that are unable to achieve treatment goals on these medications and also experience high recurrence rates. Early initiation of these therapies is key; starting treatment later in the disease is associated with poorer outcomes (Aarts, 2021).

Overall, there remains a high unmet need for treatments for HS (Chadha, 2023). Many of the antibiotics and immunomodulatory agents have significant adverse effects, sometimes considered to be worse than the disease itself. Patients also complain about the pain and odor accompanying flares of HS. Patients frequently need psychiatric care to deal with the mental health burdens of the disease (depression, anxiety, physical pain, odor, impaired sentimental and sexual life; Machado, 2019).

### Importance of TGF-β/ALK-5 in HS

TGF-β has been suggested to play a role in HS pathogenesis and has been identified in HS lesional skin (Dajnoki, 2022). Sinus tract or tunnel formation during disease progression involves the presence of epithelial strands in the dermis, an imbalance of matrix metalloproteases (MMPs), tissue inhibitors of metalloprotease (TIMPs) and increased activity of all three isoforms of TGF-β (Vossen, 2018). The increase in TGF-β may be due to dysregulation in T-cell cytokine production of IL-17. IL-17 plays a role in TGF-β-induced inflammation and TGF-β is required for Th17 cells to differentiate from naïve T-cells. A recent scRNA Seq study of HS surgical excision samples found that TGF-β was predominantly found in T-cells, B-cells and plasma cells (Smith, 2022). As TFG-β is able to stimulate extracellular matrix production and fibrosis, it is likely that the development of tunnels (a hallmark of HS) and scarring in HS is closely related to chronic activation of TGF-β signaling. TFG-β may act as a link between the innate and adaptive immune responses by linking B cells and macrophages and subsequently interacting with known pathogenic cytokines. Thus, inhibition of ALK-5 kinase and TGF-β signaling would be expected to potentially slow disease progression and prevent HS scarring.

### Fat Remodeling Disorders

The lipid storage capability of fat cells plays a critical role in systemic energy homeostasis. For proper lipid storage, lipogenesis is associated with the establishment of a corresponding extracellular matrix (ECM) to support adipocyte expansion and differentiation. Recent transcriptome analyses identified TGF-β1 as a critical link to these ECM supported remodeling processes (Toyoda, 2022). Feeding events (especially after fasting) were found to increase TGF-β1 in adipocytes but not in liver or muscle tissue. Conversely, pharmacological inhibition of TGF-β1 signaling using receptor antagonists (SB431542), blockade of downstream receptor signaling (SMAD3 KO) and neutralization of circulating TGF-β1 (antibodies) were reported to reduce adipose tissue content and adipocyte cell size in animal models. In vitro studies using 3T3-L1 adipocytes suggest that TGF-β effects are cell autonomous, involve autocrine/paracrine signaling and implicate matrix metalloprotease and disintegrin (MMP-ADAM) pathways.

A number of medical and cosmetic skin disorders involve over-production of adipose (fat) in the dermal and/or subcutaneous layers. While many of these can be treated surgically or by medical devices (e.g., radiofrequency, lasers, CoolSculpting®), there is precedent for treating some of these disorders via agents that promote lipolysis. For example, injectable deoxycholate (DC; Kybella®, Kythera Biopharmaceuticals), a secondary bile acid involved in fat emulsification, has been approved to treat submental fat (SMF), the tendency to develop a fatty pocket under the chin. Other non-approved derivatives of DC that are also co-formulated with potential synergistic agents like phosphatidylcholine (e.g., Aqualyx®, Lipodissolve®, Lipostabil) have also been assessed for SMF. These agents are also being explored for other off-label indications including lipomas, xanthelasmas, paradoxical adipose hyperplasia, piezogenic peddle papules and HIV-associated lipohypertrophy (Liu, 2021). While encouraging efficacy has been noted in these indications, the datasets are small and further placebo controlled studies are warranted. Moreover, although injection lipolysis is effective, it does carry a significant adverse reaction profile including injection induced edema, pruritus, pain and warmth at the injection site. If severe enough, patients are inhibited from enduring further treatments to achieve the optimal result (Rittes, 2008).

### Examples of Fat Remodeling Disorders

| | | Available | |
|---|---|---|---|
| Disorder | Presentation | treatment | Comment |
| Submental Fat | Fat deposit | Deoxycholate | Edema, pain, |
| | under chin | formulations | numbness |
| | | | at injection |
| | | | site are common |
| Lipomas | Benign | Typically | Effects may be |
| | neoplasm of | surgery, | temporary. Few |
| | mature fat cells | liposuction or | studies available. |
| | | debulking. | |
| | | DC has | |
| | | shown efficacy | |
| Xanthelasma | Superficial | Excision, | Scarring, |
| | lipid deposits | electro- | ectropion |
| | present as | dessication, | and |
| | yellow-orange | laser and | dyspigmentation |
| | papules and | chemical | are site effects of |
| | plaques on | destruction | current |
| | the eyelids | (trichloroacetic | treatments. |
| | | acid) | Few studies |
| | | | available. |
| Paradoxical | Firm, non-tender | Surgery | Lack of data for |
| adipose | well demarcated | | drugs; |
| hyperplasia | areas of | | amount and |
| | increased fat | | volume of |
| | volume that | | DC cost- |
| | occur 2-4 | | prohibitive and |
| | months after | | would cause too |
| | cryolipolysis | | much discomfort |
| | therapy in | | |
| | some patients | | |
| | (0.5-0.78%) | | |
| Piezogenic | Lesions | Has been | More studies |
| pedal papules | generated by | treated with | needed |
| | pressure | DC injections | |
| | inducing | off label | |
| | herniation of | | |
| | foodpad fat | | |
| | through the | | |
| | dermis causes | | |
| | pain while | | |
| | standing or | | |
| | walking | | |
| HIV associated | Distinctive fat | Liposuction | Often caused |
| lipohypertrophy | redistribution | has not | by anti- |
| | as buffalo | been | retroviral |
| | hump, | effective, is | therapy |
| | abdominal region, | expensive | (withdrawal |
| | gynecomastia, | and has side | is not an |
| | loss of fat | effects. | option) |
| | in the mid | Localized | |
| | face and | injections | |
| | extremities | of DC are | |
| | | gaining traction | |

### Importance of TGF-β/ALK-5 in Fat Remodeling

Topical ALK-5 kinase inhibition would be expected to have a number of beneficial effects to reduce local adipose depots in the above and other skin disorders caused by disturbances of lipid metabolism. TGF-β family ligands such as TGF-β, activin, GDF and BMP family growth factors are known to induce adipogenesis and adipocyte hypertrophy (Zamani, 2011). Conversely, TGF-β family inhibitors have been shown to blunt adipogenesis via non-canonical regulation of SMAD pathways (Aykul, 2021). Additionally, inhibition of the TGF-β pathway could be associated with increased thermogenesis as multiple TGF-β inhibitors (SB431542, LY2157299, A83-01 and Tanilast) induced brown fat adipogenesis in mouse fibroblasts and fat precursor cells (Tu, 2019). In addition to adipocyte reprogramming, TGF-β inhibition would also be expected to regulate the surrounding ECM to ultimately decrease adipose stores (Toyoda, 2022). Topical ALK-5 kinase inhibitors would be expected to have a more favorable side effect profile than injection lipolysis. Additionally, DC injections are associated with induration fibrosis in the skin and a combination of topical ALK-5 kinase inhibitors, along with an agent like Kybella®, could mitigate this side effect.

### Alopecia

Multiple forms of alopecia have been reported including patterned alopecia, central centrifugal cicatricial alopecia (CCCA) and alopecia areata. Patterned alopecia (PA) is the most common hair loss in men and women. Patterned hair loss is characterized by a gradual loss of terminal hair and follicular miniaturization to vellus hair fibers on the scalp in a characteristic distribution. It is a progressive condition impacting the temporal, frontal and vertex scalp in men and the central scalp in women (Tamashunas, 2021). Hormonally, hair loss has been linked to androgen excess in males, especially high dihydroxytestorsterone production, increased levels of androgen receptors, and 5-alpha reductase. First-line treatment in females includes topical minoxidil and in males, a combination of topical minoxidil and oral finasteride. Side effects of minoxidil include hair shedding, contact dermatitis, hypertrichosis and local pruritus and irritation. Finasteride is associated with erectile dysfunction (1.5%), decreased libido, gynecomastia, testicular pain and depression. Patterned alopecia is associated with psychosocial distress, decreased body image satisfaction, reduced self-esteem especially among women and diminished quality of life. Responses to current pharmacological treatments vary and there remains an unmet need to find treatments to prevent further hair loss and promote hair regrowth.

Central centrifugal cicatricial alopecia (CCCA) is a scarring alopecia that is predominantly seen in African American women (2.7 to 5.6%) with a mean age of presentation of 36 years. In CCCA, the psychosocial sequelae are even more severe due to its scarring nature (Katoulis, 2015). To combat inflammation during early acute phases of CCCA, patients are treated with doxycycline, anti-seborrheic shampoos, high potency topical steroids and intralesional triamcinolone. Once progression is stopped, topical minoxidil (5-8%) or spironolactone are used as regrowth therapy. Anti-seborrheic shampoo, mid-potency corticosteroids given orally or intralesional are given in the maintenance phase to maintain hair growth. Overall, treatments for CCCA have suboptimal efficacy and undesirable side effects.

Alopecia areata (AA) is an auto-immune hair loss disease that is characterized by chronic inflammation at the hair follicle level. The exact cause is unknown and genetic, environmental factors and immune system function have all been implicated. AA pathogenesis includes abnormal T and B lymphocytes secreting cytokines (such as IFN-γ and TNF) and the development of auto-antibodies that attack hair follicle cells, leading to hair loss (Simakou, 2019). Disruption of the immune privilege typically afforded to hair follicles instigates an immune response that results in hair loss as autoantibodies bind to antigens on the surface of hair follicle cells, forming immune complexes that further damage hair follicle cells. There are also multiple disease associations with AA including vitiligo, systemic lupus erythematosus, psoriasis, atopic dermatitis, autoimmune thyroid disease and allergic rhinitis (Chu, 2011). Intra-lesional corticosteroids (most commonly, triamcinolone) are the first-line treatments and are associated with pitting atrophy and injection pain. Topical corticosteroids such as betamethasone (0.05% as cream, lotion or ointment) are used if intra-lesional therapy is not appropriate. Systemic corticosteroids have also been used but include systemic side effects such as weight gain and increased risk for osteoporosis. As with other alopecia variants, minoxidil, methotrexate, and cyclosporine have also been used. Novel drugs include JAK inhibitors, such as baricitinib, whose side effect profile includes upper respiratory tract infections, headaches, acne, and others, and severity is generally mild to moderate. While efficacious, JAK inhibitors are also known for a high rate of relapse after discontinuation.

### Importance of TGF-β/ALK-5 in Alopecia

It is well-established that TGF-β plays an important role in the induction of catagen during the hair cycle (Hibino, 2004). Catagen is characterized by massive apoptosis of follicular epithelial cells and TGF-β2 appears in the lower part of the boundary area between the dermal papilla cells and the germinative matrix cells during anagen to catagen transition. In general, the suppression of TGF-β would be expected to promote hair growth. High levels of TGF-β 1 are present in patients with AA and positively correlate with severity, which indicated a potential causal role of TGF-β 1 in the pathogenesis of AA (El-Refaey et al 2020). Fibroproliferative genes including TGF-β2 are elevated in CCCA (Aguh, 2018). In cell-based assays, the TGF-β/ALK-5 inhibitor, TP0427736, inhibited Smad2/3 phosphorylation in A549 cells and decreased the growth inhibition of human outer root sheath cells. Two studies have demonstrated that TGF-β/ALK-5 inhibitors may be effective in androgenic alopecia (PA) models (Amada, 2013, Naruse, 2017). Topical application of TP0427736 significantly decreased Smad2 phosphorylation in mouse skin, and its repeated application suppressed the shortening of average hair follicle length during the transition from the late anagen phase to the catagen phase. Overall, these data suggest TGF-β may play a role in various etiologies of alopecia and, consequently, indicate that small molecule inhibitors of ALK-5 kinase may be a useful treatment for alopecia to promote hair growth without the side effects of existing treatments.

### Pharmaceutical Compositions:

In one aspect, the pharmaceutical composition of the invention comprise: at least one ALK-5 kinase inhibitor; a permeation enhancer; a solvent; an antioxidant; and a thickening agent. In certain embodiments, the pharmaceutical compositions of the invention may not include a preservative. In certain embodiments, the pharmaceutical compositions of the invention may include a preservative.

The invention is directed to pharmaceutical compositions of ALK-5 kinase inhibitors for treatment of various disorders related to the TGF-β receptor. Since the disorders may be on the skin, the current invention focuses on topical formulations of ALK-5 kinase inhibitors. In particular, the invention recognizes that there is a need for topical formulations comprising ALK-5 kinase inhibitors where the formulations provide sufficient dermal exposure of the ALK-5 kinase inhibitors in the formulation and where the formulation does not result in a high systemic exposure of the ALK-5 kinase inhibitors. The topical pharmaceutical compositions of the invention have several beneficial properties. These beneficial properties, include, but are not limited to: (i) optimal solubility of the one or more TGF-β receptor inhibitors in the formulation, (ii) high permeability of the one or more TGF-β receptor inhibitors, (iii) low systemic exposure of the one or more TGF-3 receptor inhibitors, (iv) long shelf stability of the pharmaceutical composition, (v) low skin irritation caused by the pharmaceutical composition by the one or more TGF-β receptor inhibitors or any other excipient in the formulation, (vi) optimal ratio of dermal deposition of one or more TGF-β receptor inhibitors as compared to the systemic exposure of the drug, (vii) sustained level of the exposure of one or more TGF-β receptor inhibitors in the dermal depot and optimal pharmacodynamics, and (ix) ease of application of the drug with optimal spreading properties on the skin. The pharmaceutical compositions of the invention may have one or more of these properties.

Preferably, administration of the pharmaceutical compositions of the invention to the patient does not lead to any unacceptable skin irritation upon administration of a therapeutically effective dose of the pharmaceutical composition.

In certain embodiments, the pharmaceutical composition has a high dermal penetration rate to enable faster absorption through the skin when the pharmaceutical composition is applied to the skin. In certain embodiments, the pharmaceutical composition has a high dermal deposition rate to reduce the systemic circulation of the ALK-5 kinase inhibitor. The dermal deposition refers to the accumulation of the ALK-5 kinase inhibitor on the skin, dermis, and the lower connective tissues. More specifically, the pharmacodynamics of the pharmaceutical compositions of the invention lead to the ALK-5 kinase inhibitor to be in the dermal tissue for the duration to have a therapeutically significant ALK-5 kinase inhibition. In certain preferred embodiments, the pharmaceutical compositions of the invention have a sufficient steady-state concentration in the dermal tissue upon once or twice daily application of the pharmaceutical composition.

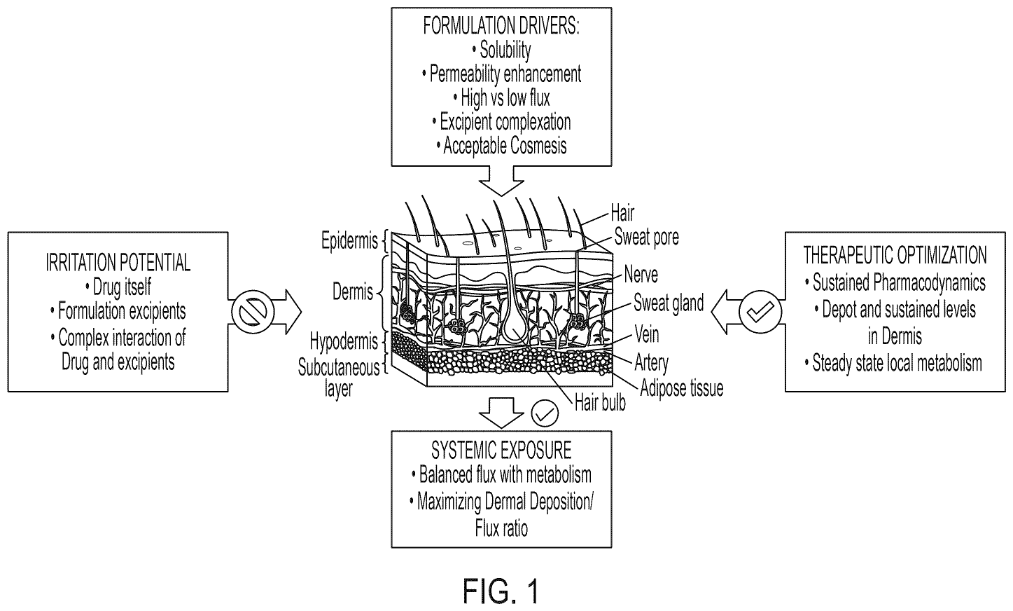

FIG. 1 provides an overview of the complexity and the relevant factors in development of topical pharmaceutical compositions. A discussion of the factors listed in FIG. 1 are also provided below. Importantly, all these factors have secondary and tertiary interactions to influence the pharmacokinetics and physical characteristics of a pharmaceutical composition. Notably, the influence of these factors over the properties of the pharmaceutical compositions is difficult to predict as a result of the complexity and the interplay involved between several competing properties of the ingredients of pharmaceutical compositions. As discussed in this application, the pharmaceutical compositions of the invention have surprising properties, especially the difference in dermal deposition rates between the various formulations listed herein. Moreover, the invention provides the characterization of pharmacokinetic properties observed in the course of conducting in vitro and in vivo experiments.

In particular, the invention recognizes that for compositions to be used by patients such as, for example, scleroderma patients, the pharmaceutical compositions should be moisturizing, moderately cooling and/or non-oily/sticky. These pharmaceutical compositions are preferred therapeutically and cosmetically as the patients suffering from diseases or disorders such as scleroderma may have highly sensitive skin and the application of compositions on the affected area may be often painful due to fibrosis. In addition, for treatment of patients suffering from diseases or disorders such as scleroderma and/or keloid scars, the pharmaceutical composition should have the ability to cover the affected areas effectively and to rub in rapidly. The pharmaceutical compositions of the invention will have an optimal balance in moisturizing and drying of the composition after application on the skin of the patient.

The pharmaceutical compositions of the invention are commercially and cosmetically acceptable. The compositions may have a moisturizing and moderately cooling effect on the skin but without causing drying of the skin. The pharmaceutical compositions of the invention have rapid absorption (i.e., “rub-in”), and are non-greasy or sticky after the application of the composition on the skin. In certain embodiments, the pharmaceutical composition is color and odor neutral. The pharmaceutical compositions minimize redness and/or irritation of the skin upon application of the composition to the skin. In certain embodiments, the pharmaceutical compositions of the invention will have minimal interaction with clothes of the patient. This is important to avoid the unintentional staining, bleaching, or oxidation of the textiles with which the pharmaceutical compositions contacts.

In certain embodiments, the pharmaceutical composition is stable under storage conditions. In one aspect, the pharmaceutical composition comprises one or more ALK-5 kinase inhibitors and one or more pharmaceutically acceptable excipients. The excipients used in the pharmaceutically acceptable compositions of the invention do not have adverse interactions with the ALK-5 kinase inhibitor in the compositions.

In certain embodiments, the pharmaceutical composition is applied to the patient's skin at an application rate of at least 2 μl/cm² or at least 3 μl/cm² of the skin of the patient.

In certain embodiments, the pharmaceutical composition is applied to more than 5% of the patient's body surface area (BSA). In one embodiment, the pharmaceutical composition is applied to more than 6% of the patient's body surface area. In one embodiment, the pharmaceutical composition is applied to more than 7% of the patient's body surface area. In one embodiment, the pharmaceutical composition is applied to more than 8% of the patient's body surface area. In one embodiment, the pharmaceutical composition is applied to more than 9% of the patient's body surface area. In one embodiment, the pharmaceutical composition is applied to at least 10% of the patient's body surface area.

In certain embodiments, the pharmaceutical composition is applied to at least 5% of the patient's body surface area up to less than 30% of the patient's body surface area. In one embodiment, the pharmaceutical composition is applied to at least 5% of the patient's body surface area up to 20% of the patient's body surface area. In one embodiment, the pharmaceutical composition is applied to at least 5% of the patient's body surface area up to 15% of the patient's body surface area. In one embodiment, the pharmaceutical composition is applied to is applied to at least 10% of the patient's body surface area up to less than 30% of the patient's body surface area. In one embodiment, the pharmaceutical composition is applied to at least 1000 of the patient's body surface area up to 200% of the patient's body surface area.

Table 1 below lists preferred target pharmacokinetic profile of the pharmaceutical compositions of the invention.

TABLE 1
Formulation Target Pharmacokinetics:

| Parameter | Target Product Profile |
|---|---|
| Formulation | A semi-solid formulation, balanced |
| type | drying-moisturizing properties |
| Dose | Ideally once or maximum twice a day application |
| Initial Target | 0.1% to 2%. |
| concentration | The maximum estimated human |
| | therapeutic dose of topically |
| | administered exemplary ALK-5 |
| | kinase inhibitor is estimated in the range |
| | of 1-2 g of formulations, if applied at 1% over a |
| | 200 cm² body surface area. This results in 0.03-0.60 |
| | mg/kg/day assuming 10% absorption. |
| Formulation | Drug release and drug permeation |
| Performance | into the skin preferably to |
| | concentrate within the dermis and the connective |
| | tissue immediately below the dermis. |
| | Systemic exposure should be |
| | minimized while simultaneously |
| | delivering optimal drug concentrations |
| | to the skin tissues and |
| | dermis in particular. |
| Formulation | Cosmetically and commercially |
| aesthetics/ | acceptable with a moisturizing |
| patient | and moderately cooling effect on |
| Acceptability | the skin but without causing |
| | drying of the skin. It should be |
| | easy to apply or spread on the |
| | skin, have rapid absorption, be |
| | non-greasy/tacky post rub-in, be |
| | color and odor neutral and minimize |
| | redness or irritation of the |
| | skin on application. Interaction with |
| | clothing is important and |
| | the preparation should not stain |
| | or bleach (oxidize) textiles with |
| | which it will come into contact. |
| | The composition may be a |
| | cream, gel, or lotion |
| Packaging | Preferably a tube or pump, etc. |
| description | from which suitable clinical |
| | amounts can be applied and |
| | which also provides acceptable |
| | physical and chemical stability. |

In one aspect, the invention provides a pharmaceutical composition for topical application, the pharmaceutical composition comprising: ALK-5 kinase inhibitor and a solvent. The pharmaceutical compositions of the invention may further comprise a permeation enhancer. The pharmaceutical compositions of the invention may further comprise a solvent. The pharmaceutical compositions of the invention may further comprise a preservative. The pharmaceutical compositions of the invention may further comprise an antioxidant. The pharmaceutical compositions of the invention may further comprise a thickening agent.

In certain embodiments, the pharmaceutical composition of the invention is a cream or a topical gel. In certain embodiments, the pharmaceutical composition of the invention is a cream. In certain embodiments, the pharmaceutical composition of the invention is a topical gel. In certain embodiments, the pharmaceutical composition is designed for optimal administration on the skin of the user. Such a composition has optimal spreading properties on the skin of the patient and sufficient thickness so that the composition does not run off from the skin prior to getting absorbed.

### ALK-5 Kinase Inhibitors

There are several small molecule drugs have been developed to inhibit ALK-5 kinase activity. Like other kinase inhibitor development, these inhibitors are designed to bind to the ATP-binding domain of the ALK-5 kinase.

The ALK-5 kinase inhibitor may be any ALK-5 kinase inhibitor. Preferably, the ALK-5 kinase inhibitor is a small molecule inhibitor. The ALK-5 kinase inhibitor may be any of the ALK-5 kinase inhibitors disclosed in any of: U.S. Pat. No. 7,964,612 (incorporated by reference in its entirety), U.S. Pat. No. 8,455,512 (incorporated by reference in its entirety), U.S. Pat. No. 9,938,289 (incorporated by reference in its entirety), U.S. Pat. No. 9,090,625 (incorporated by reference in its entirety), and/or U.S. Pat. No. 9,260,450 (incorporated by reference in its entirety).

In one aspect, the ALK-5 kinase inhibitor is represented by a compound of Formula I:

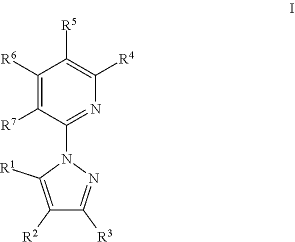

or a pharmaceutically acceptable salt thereof, wherein:
R¹ of Formula I is a thieno[3,2-c]pyridinyl, a thieno[3,2-b]pyridinyl, a thieno[2,3-c]pyridinyl, or a thieno[2,3-b]pyridinyl, each of which may be optionally substituted with one to three substituents each independently selected from the group consisting of: —C₁-C₃-alkyl, —(C₁-C₃-alkyl)-S—(C₁-C₃-alkyl), —S—C₁-C₃-alkyl, —(C₁-C₃-alkyl), —O—(C₁-C₃-alkyl), —O—C₁C₃-alkyl, —C(O)O—C₁-C₃-alkyl, —C(O)O—H, —C(O)NR³⁰R³¹, halo, —CN, —OH, wherein R³⁰ and R³¹ are each independently selected from the group consisting of: H, and —C₁-C₃ alkyl-OH, —C₁-C₃-alkyl, halo, and —O—C₁-C₃-alkyl;
R² and R³ of Formula I are independently selected from the group consisting of: hydrogen, —C₁-C₃-alkyl, —(C₁-C₃-alkyl)-S—(C₁-C₃-alkyl), —S—C₁-C₃-alkyl, —(C₁-C₃-alkyl)-O—(C₁-C₃-alkyl), —O—C₁-C₃-alkyl, —C(O)O—H, —C(O)NH₂, —C(O)N(C₁-C₃ alkyl), —C(O)N(C₁-C₃ alkyl)₂, halo, —CN, —OH, and a C₃-C₆-cycloalkyl; or R² and R³ may be taken together to form a 5 or 6-membered heteroaryl, a phenyl, a C₄-C₆-cycloalkyl, or a 4-6-membered heterocycloalkyl, wherein said C₄-C₆-cycloalkyl or 4-6-membered heterocycloalkyl may be optionally substituted with one to three substituents independently selected from halo, —OH, oxo, and —C₁-C₃ alkyl, wherein said 5 or 6-membered heteroaryl, or phenyl may be optionally substituted with one to three substituents independently selected from halo, —CN, —OH, —O—C₁-C₃ alkyl and —C₁-C₃ alkyl; and
R⁴, R⁵, R⁶, and R⁷ of Formula I are independently selected from the group consisting of: H, —OH, C₃-cycloalkyl, —C₁-C₃-alkyl, —(C₁-C₃-alkyl)-S—(C₁-C₃-alkyl), —S—C₁-C₃-alkyl, —(C₁-C₃-alkyl)-O—(C₁-C₃-alkyl), —O—C₁-C₃-alkyl, —C(O)O—C₁-C₃-alkyl, —C(O)O—H, —C(O)NH₂, —C(O)NH(C₁-C₃-alkyl), —C(O)NH(O—C₁-C₃ alkyl), —C(O)N(H)(halo), —C(O)N(C₁-C₃alkyl)₂, —C(O)N(C₁-C₃ alkyl)(O—C₁C₃ alkyl), —C(O)N((C₁C₃ alkyl)(halo), —C(O)N(O—C₁-C₃ alkyl)₂, —C(O)N(O—C₁-C₃ alkyl)(halo), —C(O)N(halo)₂, halo, —CN, and —OH.

In certain embodiments, R¹ of Formula I is a thieno[3,2-c]pyridinyl, which may be optionally substituted as specified herein. The positions of a thieno[3,2-c]pyridine are numbered as follows:

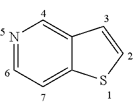

A thieno[3,2-c]pyridinyl is a monovalent radical of thieno[3,2-c]pyridine. Thus, in certain embodiments of the present invention, are compounds of Formula II:

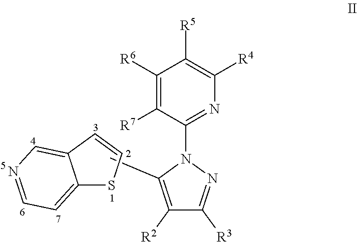

wherein R¹, R², R³, R⁴, R⁵, R⁶, and R⁷ have any of the values specified herein, and wherein the thieno[3,2-c]radical is attached at any of positions 2, 3, 4, 6, or 7.

In certain embodiments, R¹ is a thieno[2,3-c]pyridinyl, which may be optionally substituted as specified. The positions of a thieno[2,3-c]pyridine are numbered as follows:

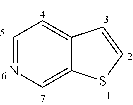

A thieno[2,3-c]pyridinyl is a monovalent radical of thieno[2,3-c]pyridine. Thus, in certain embodiments of the present invention, are compounds of Formula III:

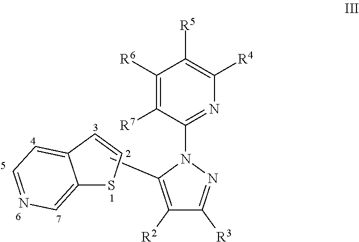

wherein R¹, R², R³, R⁴, R⁵, R⁶, and R⁷ have any of the values specified herein, and wherein the thieno[2,3-c]radical is attached at any of positions 2, 3, 4, 5, or 7.

In certain embodiments, R¹ is a thieno[2,3-b]pyridinyl, which may be optionally substituted as specified herein. The positions of a thieno[2,3-b]pyridine are numbered as follows:

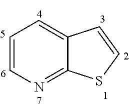

A thieno[2,3-b]pyridinyl is a monovalent radical of thieno[2,3-c]pyridine. Thus, in certain embodiments of the present invention, are compounds of Formula IV:

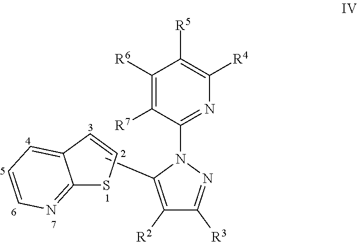

wherein R¹, R², R³, R⁴, R⁵, R⁶, and R⁷ have any of the values specified herein, and wherein the thieno[2,3-b]radical is attached at any of positions 2, 3, 4, 5, or 6.

In certain embodiments, R¹ is a thieno[3,2-b]pyridinyl, which may be optionally substituted as specified herein. The positions of a thieno[3,2-b]pyridine are numbered as follows:

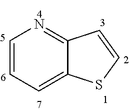

A thieno[3,2-b]pyridinyl is a monovalent radical of thieno[3,2-c]pyridine. Thus, in certain embodiments of the present invention, are compounds of Formula V:

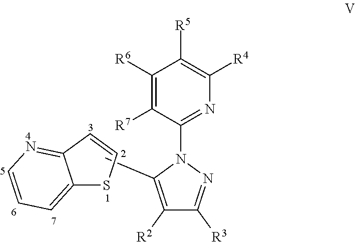

wherein R¹, R², R³, R⁴, R⁵, R⁶, and R⁷ have any of the values specified herein, and wherein the thieno[3,2-b]radical is attached at any of positions 2, 3, 5, 6, or 7.

In certain embodiments, R¹ is a thieno[3,2-c]pyridinyl or a thieno[2,3-c]pyridinyl.

In certain embodiments, R¹ is a thieno[3,2-c]pyridinyl or a thieno[2,3-c]pyridinyl, which may be optionally substituted with one to three substituents each independently selected from the group consisting of: C₁-C₃-alkyl, —(C₁-C₃-alkyl)-S—(C₁-C₃-alkyl), —S—C₁-C₃-alkyl, —(C₁-C₃-alkyl)-O—(C₁-C₃-alkyl), —O—C₁-C₃-alkyl, —C(O)O—C₁-C₃-alkyl, —C(O)O—H, —C(O)NR³⁰R³¹, halo, —CN, —OH, wherein R³⁰ and R³¹ are each independently selected from the group consisting of: H, and C₁-C₃ alkyl-OH, C₁-C₃-alkyl, halo, and —O—C₁-C₃-alkyl;

In certain embodiments, R¹ is a thieno[3,2-c]pyridinyl or a thieno[2,3-c]pyridinyl, which may be optionally substituted with one to three substituents each independently selected from the group consisting of: —OH, C₁-C₃ alkyl, halo, and —O—C₁-C₃ alkyl.

In certain embodiments, R² and R³ are taken together to form a C₄-C₆-cycloalkyl, or a 4-6-membered heterocycloalkyl, wherein said C₄-C₆-cycloalkyl or 4-6-membered heterocycloalkyl may be optionally substituted with one to three substituents independently selected from oxo and C₁-C₃ alkyl. In other embodiments, R² and R³ are taken together to form a C₅-cycloalkyl, or a 4-6-membered heterocycloalkyl, wherein said 4-6-membered heterocycloalkyl is selected from the group consisting of: a tetrahydrofuranyl, a tetrahydrothienyl, a imidazolidinyl, an oxazolidinyl, an imidazolinyl, an isoxazolidinyl, and a pyrrolidinyl. In other embodiments, R² and R³ are taken together to form a C₅-cycloalkyl and R¹ is a thieno[3,2-c]pyridinyl or a thieno[2,3-c]pyridinyl.

In certain embodiments, a compound of the present invention is (2-(6-methylpyridin-2-yl)-2,4,5,6-tetrahydrocyclopenta[c]pyrazol-3-yl)thieno[3,2-c]pyridine, or a pharmaceutically acceptable salt thereof.

In certain embodiments, a compound of the present invention is 2-(2-(6-methylpyridin-2-yl)-2,4,5,6-tetrahydrocyclopenta[c]pyrazol-3-yl)thieno[2,3-c]pyridine, or a pharmaceutically acceptable salt thereof.

In other embodiments, R² and R³ are independently selected from the group consisting of: hydrogen, C₁-C₃-alkyl, —(C₁-C₃-alkyl)-O—(C₁-C₃-alkyl), —O—C₁-C₃-alkyl, —C(O)O—C₁-C₃-alkyl, —C(O)O—H, —C(O)NH₂, —C(O)NH(C₁-C₃ alkyl), —C(O)N(C₁-C₃ alkyl)₂, halo, —CN, —OH, and a C₃-C₆cycloalkyl. In particular embodiments, R² and R³ are independently selected front the group consisting of: hydrogen, and C₁-C₃ alkyl. In more particular embodiments, R² is C₁-C₂ alkyl and R³ is hydrogen.

In certain embodiments, R⁵, R⁶, and R⁷ are H, and R⁴ is C₁-C₃-alkyl. In yet other embodiments, R⁴ is methyl. In certain embodiments, R⁵, R⁶, and R⁷ are H, and R⁴ is methyl.

In certain embodiments, R¹ is a thieno[3,2-c]pyridinyl or a thieno[2,3-c]pyridinyl, which may be optionally substituted with one to three substituents each independently selected from the group consisting of: —OH, C₁-C₃ alkyl, halo, and —O—C₁-C₃ alkyl. In certain embodiments, R¹ is a thieno[3,2-c]pyridin-2-yl or a thieno[2,3-c]pyridin-2-yl, which may be optionally substituted with one to three substituents each independently selected from the group consisting of: —OH, C₁-C₃ alkyl, halo, and —O—C₁-C₃ alkyl. In certain embodiments, R¹ is a thieno[3,2-c]pyridine-2-yl, which may be optionally substituted with one to three substituents each independently selected from the group consisting of: —OH, C₁-C₃ alkyl, halo, and —O—C₁-C₃ alkyl. In particular embodiments, R¹ is thieno[3,2-c]pyridinyl-2yl.

In certain embodiments, R⁵, R⁶, and R⁷ are H; R⁴ is a C₁-C₃-alkyl; and R¹ is a thieno[3,2-c]pyridinyl or a thieno[2,3-c]pyridinyl, which may be optionally substituted with one to three substituents each independently selected from the group consisting of: —OH, C₁-C₃ alkyl, halo, and —O—C₁-C₃ alkyl. In certain embodiments, R⁵, R⁶, and R⁷ are H; R⁴ is methyl; and R¹ is a thieno[3,2-c]pyridinyl or a thieno[2,3-c]pyridinyl, which may be optionally substituted with one to three substituents each independently selected from the group consisting of: —OH, C₁-C₃ alkyl, halo, and —O—C₁-C₃ alkyl. In certain embodiments, R⁵, R⁶, and R⁷ are H; R⁴ is methyl; and R¹ is a thieno[3,2-c]pyridinyl or a thieno[2,3-c]pyridinyl. In certain embodiments, R⁵, R⁶, and R⁷ are H; R⁴ is methyl; and R¹ is a thieno[2,3-c]pyridinyl. In certain embodiments, R⁵, R⁶, and R⁷ are H; R⁴ is methyl; and R¹ is a thieno[3,2-c]pyridinyl.

In certain embodiments, R⁵, R⁶, and R⁷ are H; R⁴ is methyl; R¹ is a thieno[2,3-c]pyridinyl; and R² and R³ are independently selected from the group consisting of: hydrogen, and C₁-C₃ alkyl.

In certain embodiments, R⁵, R⁶, and R⁷ are H; R⁴ is methyl; R¹ is a thieno[3,2-c]pyridinyl; and R² and R³ are independently selected from the group consisting of: hydrogen, and C₁-C₃ alkyl.

In certain embodiments, R⁵, R⁶, and R⁷ are H; R⁴ is methyl; R¹ is a thieno[2,3-c]pyridin-2-yl; and R² and R³ are independently selected from the group consisting of: hydrogen, and C₁-C₃alkyl.

In certain embodiments, R⁵, R⁶, and R⁷ are H; R⁴ is methyl; R¹ is a thieno[3,2-c]pyridin-2-yl; and R² and R³ are independently selected from the group consisting of: hydrogen, and C₁-C₃alkyl.

Examples of compounds of Formula I include:
2-[1-(6-methylpyridin-2-yl)-1H-pyrazol-5-yl]thieno[3,2-c]pyridine;
2-[3,4-dimethyl-1-(6-methylpyridin-2-yl)-1H-pyrazol-5-yl]thieno[3,2-c]pyridine;
2-[3-methyl-1-(6-methylpyridin-2-yl)-1H-pyrazol-5-yl]thieno[3,2-c]pyridine;
2-[3-ethyl-1-(6-methylpyridin-2-yl)-1H-pyrazol-5-yl]thieno[3,2-c]pyridine;
2-[4-ethyl-1-(6-methylpyridin-2-yl)-1H-pyrazol-5-yl]thieno[3,2-c]pyridine; and pharmaceutically acceptable salts thereof.

Another example of a compound of FormulaI is 2-[4-methyl-1-(6-methylpyridin-2-yl)-1H-pyrazol-5-yl]thieno[3,2-c]pyridine; and pharmaceutically acceptable salts thereof.

In certain embodiments, the compound of the invention is selected from the group consisting of:

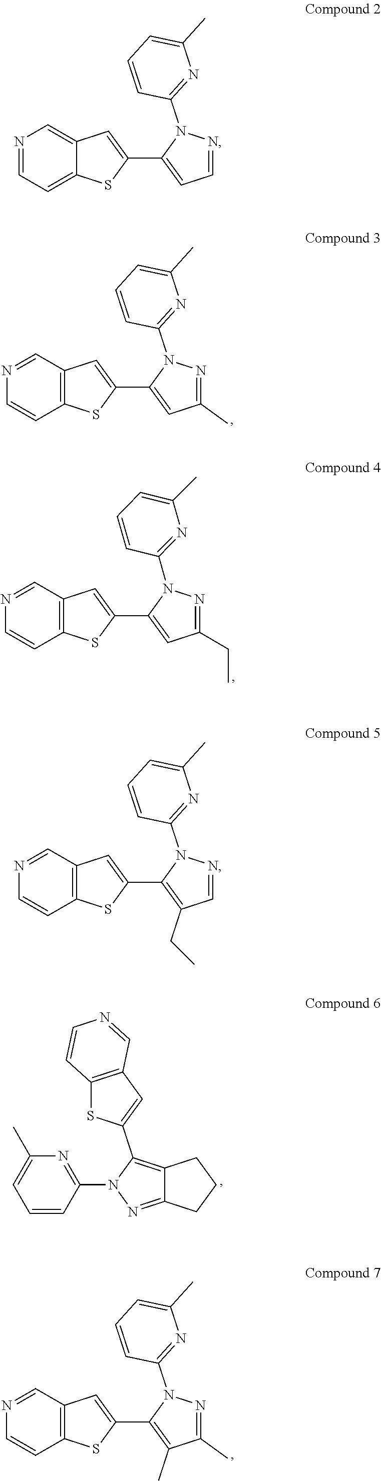 [SMILES: Cc1cccc(-n2nccc2-c2cc3cnccc3s2)n1 ; Cc1cccc(-n2nc(C)cc2-c2cc3cnccc3s2)n1 ; CCc1cc(-c2cc3cnccc3s2)n(-c2cccc(C)n2)n1 ; CCc1cnn(-c2cccc(C)n2)c1-c1cc2cnccc2s1 ; Cc1cccc(-n2nc3c(c2-c2cc4cnccc4s2)CCC3)n1 ; Cc1cccc(-n2nc(C)c(C)c2-c2cc3cnccc3s2)n1]

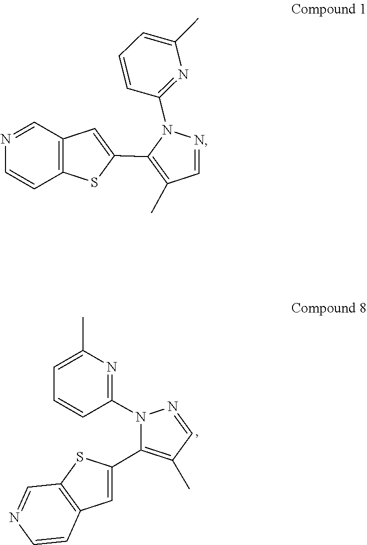 [SMILES: Cc1cccc(-n2ncc(C)c2-c2cc3cnccc3s2)n1 ; Cc1cccc(-n2ncc(C)c2-c2cc3ccncc3s2)n1]

and 2-[4-methyl-2-(6-methyl-pyridin-2-yl)-2H-pyrazol-3-yl]-thieno[3,2-c]pyridine, and pharmaceutically acceptable salts thereof.

In one embodiment, Compound 1 is shown as

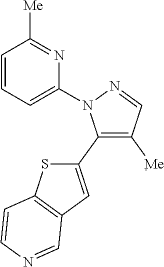 [SMILES: Cc1cccc(-n2ncc(C)c2-c2cc3cnccc3s2)n1]

In another aspect, the ALK-5 kinase inhibitor is represented by a compound of Formula VI:

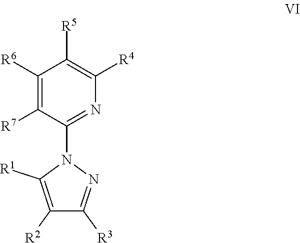

or a pharmaceutically acceptable salt thereof, wherein:
R¹ of Formula VI is selected from the group consisting of thieno[3,2-c]pyridinyl, thieno[3,2-b]pyridinyl, thieno[2,3-c]pyridinyl, and thieno[2,3-b]pyridinyl; wherein each may be optionally substituted with one to three substituents each independently selected from the group consisting of C₁-C₃-alkyl, —(C₁-C₃-alkyl)S(C₁-C₃-alkyl), —S(C₁-C₃-alkyl), —(C₁-C₃-alkyl)O(C₁-C₃-alkyl), —O(C₁-C₃-alkyl), —C(═O)O(C₁-C₃-alkyl), —CO₂H, —C(═O)NR⁸R⁹, halo, —CN, and —OH;
R² and R³ of Formula VI are independently selected from the group consisting of H, C₁-C₃-alkyl, —(C₁-C₃-alkyl)S(C₁-C₃-alkyl), —S(C₁-C₃-alkyl), —(C₁-C₃-alkyl)O(C₁-C₃-alkyl), —O(C₁-C₃-alkyl), —C(═O)O(C₁-C₃-alkyl), —CO₂H, —C(═O)NR¹⁰R¹¹, halo, —CN, —OH, and C₃-C₆-cycloalkyl;
alternatively, R² and R³ of Formula VI may be taken together to form a 5-6-membered heteroaryl, phenyl, a C₄-C₆-cycloalkyl, or a 4-6-membered heterocycloalkyl; wherein C₄-C₆-cycloalkyl and 4-6-membered heterocycloalkyl may be optionally substituted with one to three substituents independently selected from the group consisting of halo, —OH, oxo, and C₁-C₃ alkyl; wherein 5-6-membered heteroaryl and phenyl may be optionally substituted with one to three substituents independently selected from the group consisting of halo, —CN, —OH, —O(C₁-C₃ alkyl), and C₁-C₃ alkyl;
R⁴, R⁵, R⁶, and R⁷ of Formula VI are selected from the group consisting of H, C₃-cycloalkyl, C₁-C₃-alkyl, —(C₁-C₃-alkyl)S(C₁-C₃-alkyl), —S(C₁-C₃-alkyl), —(C₁-C₃-alkyl)O(C₁-C₃-alkyl), —O(C₁-C₃-alkyl), —C(═O)O(C₁-C₃-alkyl), —CO₂H, —C(═O)NR¹²R¹³, halo, —CN, —OH;
R⁸ and R⁹ of Formula VI are each independently selected from the group consisting of H, and —(C₁-C₃ alkyl)OH, C₁-C₃-alkyl, halo, and —O(C₁-C₃-alkyl);
R¹⁰ and R¹¹ of Formula VI are each independently selected from the group consisting of H and C₁-C₃ alkyl; and
R¹² and R¹³ of Formula VI are each independently selected from the group consisting of H, C₁-C₃ alkyl, halo, and —O(C₁-C₃-alkyl).

In certain embodiments, the compound of Formula VI is selected from the group consisting of:

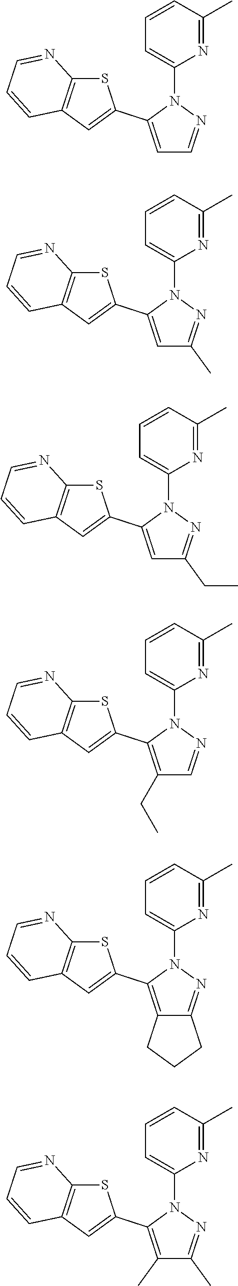

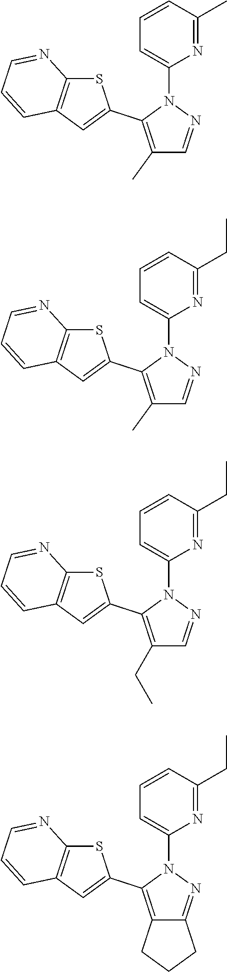 [SMILES: Cc1cccc(-n2ncc(C)c2-c2cc3cccnc3s2)n1 ; CCc1cccc(-n2ncc(C)c2-c2cc3cccnc3s2)n1 ; CCc1cccc(-n2ncc(CC)c2-c2cc3cccnc3s2)n1 ; CCc1cccc(-n2nc3c(c2-c2cc4cccnc4s2)CCC3)n1]

or a pharmaceutically acceptable salt thereof.

In certain embodiments, the compound of Formula VI is selected from the group consisting of:

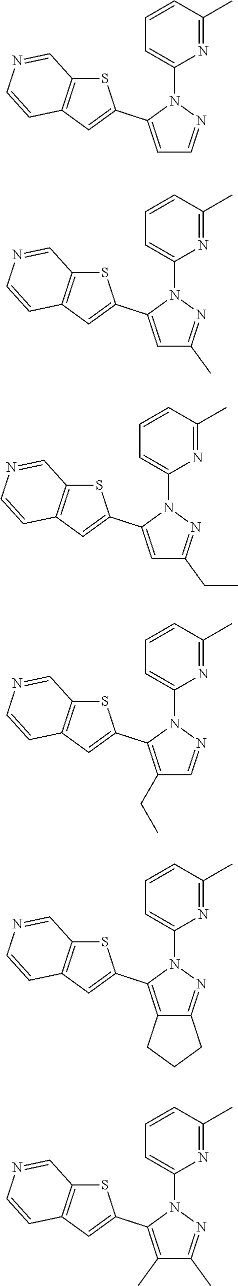

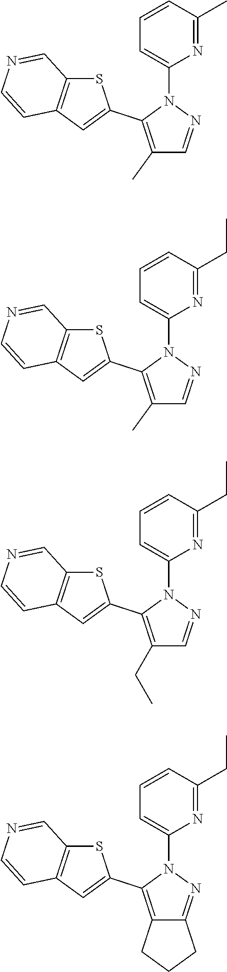 [SMILES: Cc1cccc(-n2ncc(C)c2-c2cc3ccncc3s2)n1 ; CCc1cccc(-n2ncc(C)c2-c2cc3ccncc3s2)n1 ; CCc1cccc(-n2ncc(CC)c2-c2cc3ccncc3s2)n1 ; CCc1cccc(-n2nc3c(c2-c2cc4ccncc4s2)CCC3)n1]

or a pharmaceutically acceptable salt thereof.

In certain embodiments, the compound of Formula VI is selected from the group consisting of:

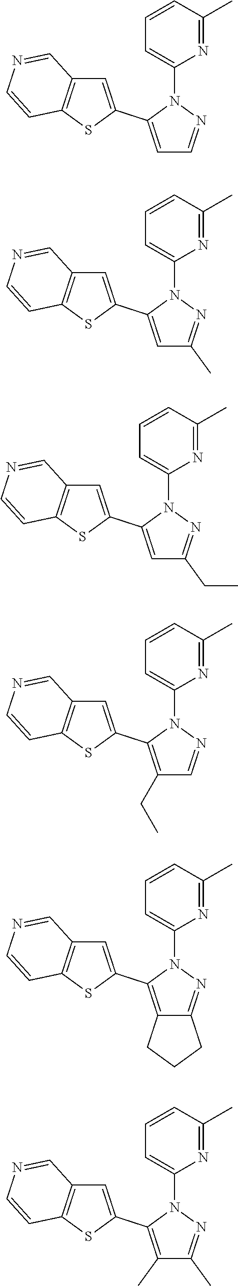

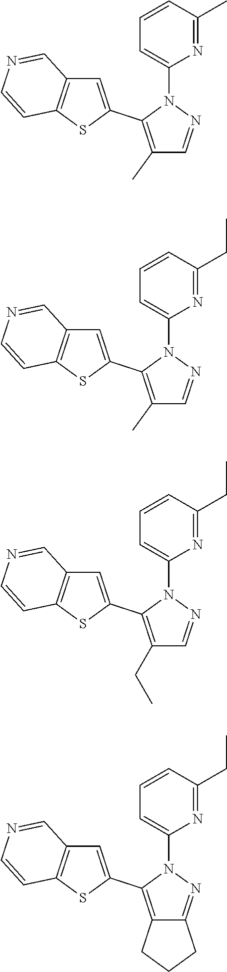 [SMILES: Cc1cccc(-n2ncc(C)c2-c2cc3cnccc3s2)n1 ; CCc1cccc(-n2ncc(C)c2-c2cc3cnccc3s2)n1 ; CCc1cccc(-n2ncc(CC)c2-c2cc3cnccc3s2)n1 ; CCc1cccc(-n2nc3c(c2-c2cc4cnccc4s2)CCC3)n1]

or a pharmaceutically acceptable salt thereof.

In certain embodiments, the compound of Formula VI is selected from the group consisting of:

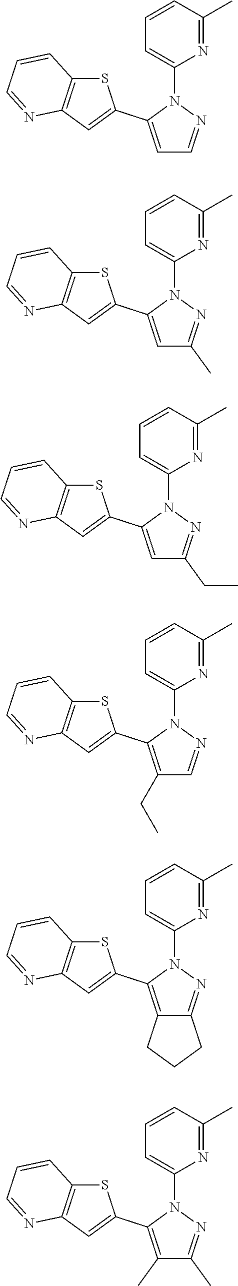

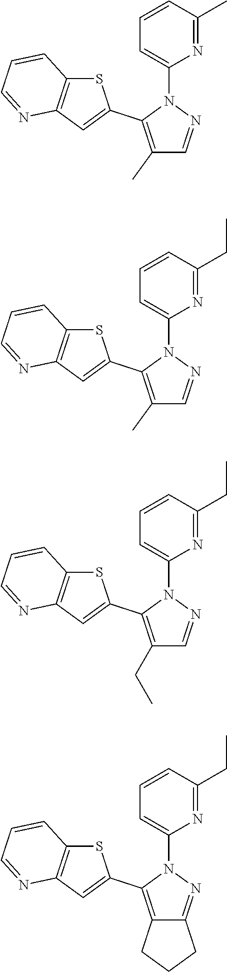 [SMILES: Cc1cccc(-n2ncc(C)c2-c2cc3ncccc3s2)n1 ; CCc1cccc(-n2ncc(C)c2-c2cc3ncccc3s2)n1 ; CCc1cccc(-n2ncc(CC)c2-c2cc3ncccc3s2)n1 ; CCc1cccc(-n2nc3c(c2-c2cc4ncccc4s2)CCC3)n1]

or a pharmaceutically acceptable salt thereof.

In an embodiment, the compound of Formula VI is

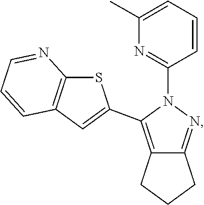 [SMILES: Cc1cccc(-n2nc3c(c2-c2cc4cccnc4s2)CCC3)n1]

or a pharmaceutically acceptable salt thereof.

In another embodiment, the compound of Formula VI is

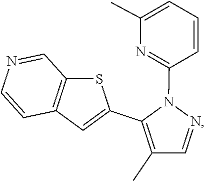 [SMILES: Cc1cccc(-n2ncc(C)c2-c2cc3ccncc3s2)n1]

or a pharmaceutically acceptable salt thereof.

In another embodiment, the compound of Formula VI is

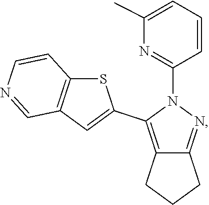 [SMILES: Cc1cccc(-n2nc3c(c2-c2cc4cnccc4s2)CCC3)n1]

or a pharmaceutically acceptable salt thereof.

In another embodiment, the compound of Formula VI is

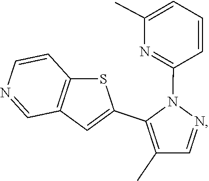 [SMILES: Cc1cccc(-n2ncc(C)c2-c2cc3cnccc3s2)n1]

or a pharmaceutically acceptable salt thereof.

The term “pharmaceutically acceptable salt” includes, for example, non-toxic salts derived from inorganic or organic acids, including, for example, but not limited to, the following acid salts: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2 naphthalenesulfonate, nicotinate, nitrate, oxalate, palmoate, pectinate, persulfate, 3 phenylpropionate, phosphate, picrate, pivalate, propionate, p-toluenesulfonate, salicylate, succinate, sulfate, tartrate, thiocyanate, and undecanoate.

The processes for preparing these compounds is described in one or more of: U.S. Pat. Nos. 7,964,612, 8,455,512, 9,938,289, 9,090,625, and/or 9,260,450, which are all incorporated by reference in their entirety.

In certain embodiments, the pharmaceutical composition comprises a therapeutically effective amount of the ALK-5 kinase inhibitor. In certain embodiments, the pharmaceutical composition comprises from about 0.05% (w/w) to about 5% (w/w) of the ALK-5 kinase inhibitor. In preferred embodiments, the pharmaceutical composition comprises from about 0.05% (w/w) to about 1% (w/w) of the ALK-5 kinase inhibitor. In certain embodiments, the pharmaceutical composition comprises from about 0.08% (w/w) to about 1.3% (w/w) of the ALK-5 kinase inhibitor. In certain embodiments, the pharmaceutical composition comprises from about 0.1% (w/w), about 0.3% (w/w), or about 1% (w/w) of the ALK-5 kinase inhibitor.

### Thickening Agents:

A thickening agent or thickener is a substance which can increase the viscosity of a liquid without substantially changing its other properties. In certain embodiments, the pharmaceutical compositions of the invention comprise thickening agents. In certain embodiments, the thickening agents in the pharmaceutical composition may be a mixture of two or more thickening agents. In certain embodiments, the thickening agent in the pharmaceutical composition is selected from the group consisting of: carbomer, methyl cellulose, sodium carboxyl methyl cellulose (NaCMC), carrageenan, colloidal silicon dioxide, trolamine, guar gum, hydroxypropyl cellulose (HPC), hydroxypropyl methyl cellulose (HPMC), gelatin, polyethylene oxide, alginic acid, sodium alginate, fumed silica, and any combination thereof. In certain embodiments, the pharmaceutical composition comprises from about 0.5% (w/w) to about 10% (w/w) of the thickener.

In certain embodiments, the pharmaceutical composition comprises from about 1% (w/w) to about 5% (w/w) of the thickener. In certain embodiments, the pharmaceutical composition comprises about 2% (w/w) of the thickener.

### Permeation Enhancers

A permeation enhancer is advantageous for increasing the permeation of the TGF-β receptor inhibitor in the skin. In certain embodiments, the pharmaceutical compositions of the invention include permeation enhancers. In certain embodiments, the permeation enhancers in the pharmaceutical composition may be a mixture of two or more permeation enhancers. In certain embodiments, the permeation enhancer in the pharmaceutical composition is selected from a group consisting of propylene glycol, ethanol, isopropyl alcohol, oleic acid, polyethylene glycol, diethylene glycol monoethyl ether, isopropyl myristate, dimethyl sulfoxide, capric acid, hexanoic acid, lauric acid, linoleic acid, linolenic acid, propionic acid, glycerol monolaurate, glycerol monooleate, glycerol monolinoleate, isopropyl isostearate, isopropyl palmitate, diethylsebacate, sorbitan monopalmitate, sorbitan oleate, sorbitan dilaurate, sorbitan trioleate, propylene glycol monolaurate, sucrose monolaurate, and any combination thereof.

Exemplary penetration enhancers include, but are not limited to, fatty acids, fatty acid esters, fatty alcohols, pyrrolidones, sulfoxides, alcohols, diols and polyols, and mixtures thereof.

Exemplary fatty acids include, but are not limited to, oleic acid, capric acid, hexanoic acid, lauric acid, linoleic acid, linolenic acid, propionic acid and vaccenic acid, and mixtures thereof.

Exemplary fatty acid esters include, but are not limited to, glycerol monolaurate, glycerol monooleate, glycerol monolinoleate, isopropyl isostearate, isopropyl palmitate, isopropyl myristate, diethylsebacate, sorbitan monopalmitate, sorbitan oleate, sorbitan dilaurate, sorbitan trioleate, propylene glycol monolaurate and sucrose monolaurate, and mixtures thereof.

Exemplary fatty alcohols include, but are not limited to, cetyl alcohol, stearyl alcohol, decanol, tridecanol, lauryl alcohol, linolenyl alcohol and oleyl alcohol, and mixtures thereof.

Exemplary pyrrolidones include, but are not limited to, N-methyl pyrrolidone, 2-pyrrolidone and N-cyclohexyl-2-pyrrolidone, and mixtures thereof.

Exemplary sulfoxides include, but are not limited to, dimethyl sulfoxide and decylmethyl sulfoxide, and mixtures thereof.

Exemplary alcohols include, but are not limited to, lower (C₁-C₆) alcohols and diethylene glycol monoethyl ether, and mixtures thereof.

Exemplary diols include, but are not limited to, 1,2-hexanediol, butylene glycol, diethylene glycol, dipropylene glycol, ethyl hexanediol, ethylene glycol, hexylene glycol, pentylene glycol, propylene glycol, propylene glycol monolaurate, tetraethylene glycol, triethylene glycol, tripropylene glycol, polyethylene glycol and polypropylene glycol, and mixtures thereof.

Exemplary polyols include, but are not limited to, butanetriol, glycerol and 1,2,6-hexanetriol, and mixtures thereof.

Suitably, the penetration enhancer is present in the composition in an amount from about 0.5% to about 40% by weight, such as from about 1% to about 20% by weight or from about 5% to about 15% by weight, based on the total weight of the composition.

In certain embodiments, the pharmaceutical composition comprises from about 10% (w/w) to about 70% (w/w) of the permeation enhancer. In certain embodiments, the pharmaceutical composition comprises from about 20% (w/w) to about 60% (w/w) of the permeation enhancer. In certain embodiments, the pharmaceutical composition comprises from about 30% (w/w) to about 50% (w/w) of the permeation enhancer.

### Antioxidants

In certain embodiments, the pharmaceutical compositions of the invention include antioxidants. In certain embodiments, the antioxidants in the pharmaceutical composition may be a mixture of two or more antioxidants. In certain embodiments, the antioxidant in the pharmaceutical composition is selected from a group a consisting of: butylated hydroxyanisole, butylated hydroxytoluene, tocopherol, propyl gallate, vitamin E, tert-butylhydroquinone and a combination thereof. In an embodiment, the antioxidant is used in conjunction with a chelating agent to prevent or minimize metal-catalyzed reactions, such as reactions catalyzed by iron, nickel, copper, magnesium, calcium, zinc or aluminum ions. In certain embodiments, the pharmaceutical composition comprises from about 0.05% (w/w) to about 0.5% (w/w) of the antioxidant. In certain embodiments, the pharmaceutical composition comprises about 0.2% (w/w) of the antioxidant.

### Preservatives

In certain embodiments, the pharmaceutical composition of the invention includes preservatives. In certain embodiments, the preservatives in the pharmaceutical composition may be a mixture of two or more preservatives. In certain embodiments, the preservative in the pharmaceutical composition is selected from a group consisting of: benzyl alcohol, imidazolidinyl urea, diazolidinyl urea, dichlorobenzyl alcohol, chloroxylenol, methyl paraben, ethyl paraben, propyl paraben, butyl paraben, phenoxyethanol, sorbic acid, benzoic acid, benzalkonium chloride, phenyl mercuric acetate, chlorobutanol, phenoxyethanol, and any combinations thereof.

Exemplary preservatives include, but are not limited to, benzyl alcohol, imidazolidinyl urea, diazolidinyl urea, dichlorobenzyl alcohol, chloroxylenol, methyl paraben, ethyl paraben, propyl paraben, butyl paraben, phenoxyethanol, sorbic acid, benzoic acid, salts thereof, and mixtures thereof.

In an embodiment, the preservative is selected from the group consisting of benzyl alcohol, phenoxyethanol and benzoic acid, and mixtures thereof.

In one embodiment, the preservative is benzyl alcohol. In another embodiment, the preservative is phenoxyethanol. In yet another embodiment, the preservative is benzoic acid.

Suitably, the preservative is present in the composition in an amount from about 0.01% to about 2% by weight, based on the total weight of the composition. In one embodiment, the preservative is present in the composition in an amount of about 0.25% by weight, based on the total weight of the composition.

In certain embodiments, the pharmaceutical composition comprises about 0.05% (w/w) to about 0.5% (w/w) of the preservative. In certain embodiments, the pharmaceutical composition comprises about 0.1% (w/w) to about 0.3% (w/w) of the preservative.

### Solvents

In certain embodiments, the pharmaceutical composition of the invention includes solvents. In certain embodiments, the solvents in the pharmaceutical composition may be a mixture of two or more solvents. In certain embodiments, the solvents may comprise an aqueous solvent and/or a non-aqueous solvent. In certain embodiments, the solvents may include a combination of one or more aqueous solvents and one or more non-aqueous solvents.

In certain embodiments, the solvent in the pharmaceutical composition is selected from a group consisting of water, hexylene glycol, propylene glycol, oleyl alcohol, propylene carbonate, mineral oil, diethylene glycol monoethyl ether, ethanol, polyethylene glycol, water, isopropanol, t-butyl alcohol, amyl alcohol, benzyl alcohol, diacetone alcohol, hexyl alcohol, tetrahydrofurfuryl alcohol, acetic acid, carboxylic acids, 1,2-hexanediol, butylene glycol, diethylene glycol, dipropylene glycol, ethyl hexanediol, ethylene glycol, propylene glycol monolaurate, tetraethylene glycol, triethylene glycol, tripropylene glycol, butyl stearate, C12-15 alkyl benzoate, C12-15 alkyl lactate, caprylic/capric triglyceride, cetearyl ethylhexanoate, diethylhexyl adipate, di-ethylhexyl succinate, diisopropyl adipate, dioctyl malate, di-PPG-2 myreth-10 adipate, di-PPG-3 myristyl ether adipate, ethyl oleate, ethylhexyl cocoate, ethylhexyl hydroxystearate, ethylhexyl palmitate, ethylhexyl pelargonate, ethylhexyl stearate, hexyl laurate, hexyldecyl laurate, an any combination thereof. In certain embodiments, the pharmaceutical composition comprises about 30% (w/w) to about 98% (w/w) of the solvent. In certain embodiments, the pharmaceutical composition comprises about 40% (w/w) to about 95% (w/w) of the solvent.

In certain embodiments, the permeation enhancer in the pharmaceutical composition is selected from a group consisting of propylene glycol, ethanol, isopropyl alcohol, oleic acid, polyethylene glycol, diethylene glycol monoethyl ether, isopropyl myristate, capric acid, hexanoic acid, lauric acid, linoleic acid, linolenic acid, propionic acid, glycerol monolaurate, glycerol monooleate, glycerol monolinoleate, isopropyl isostearate, isopropyl palmitate, diethylsebacate, sorbitan monopalmitate, sorbitan oleate, sorbitan dilaurate, sorbitan trioleate, propylene glycol monolaurate, sucrose monolaurate, and any combination thereof. In certain embodiments, the pharmaceutical composition comprises from about 10% (w/w) to about 70% (w/w) of the permeation enhancer. In certain embodiments, the pharmaceutical composition comprises from about 20% (w/w) to about 60% (w/w) of the permeation enhancer. In certain embodiments, the pharmaceutical composition comprises from about 30% (w/w) to about 50% (w/w) of the permeation enhancer.

### Surfactant

In certain embodiments, the pharmaceutical composition further comprises a surfactant. In an embodiment, the surfactant is a mixture of two or more surfactants. As used herein, a surfactant is a compound that lowers the surface tension between two liquids or between a liquid and a solid. Surfactants may also act as detergents, wetting agents, emulsifiers, foaming agents, and dispersants. As further used herein, an emulsifier is equivalent to a surfactant. The surfactant may be selected from group consisting of polysorbate 80, pemulen TR-1, Arlacel 165, castor oil, hydrogenated castor oil, propylene glycol monolaurate, caprylic triglycerides, capric triglycerides, glycerol stearate, PEG sterate, and any combinations thereof.

A surfactant's hydrophilic/lipophilic balance (HLB) describes the surfactant's affinity toward water or oil. The HLB scale ranges from 1 (totally lipophilic) to 20 (totally hydrophilic), with 10 representing an equal balance of both characteristics. Lipophilic surfactants tend to form water-in-oil (w/o) emulsions, and hydrophilic surfactants tend to form oil-in-water (o/w) emulsions. The HLB of a blend of two surfactants equals the weight fraction of surfactant A times its HLB value plus the weight fraction of surfactant B times its HLB value (weighted average).

In one embodiment, the surfactant comprises one or more non-ionic surfactants. In another embodiment, the surfactant comprises two or more non-ionic surfactants and the weighted average of the HLB values of the two or more non-ionic surfactants is from about 10 to about 20. In yet another embodiment, the surfactant comprises two or more non-ionic surfactants and the weighted average of the HLB values of the two or more non-ionic surfactants is from about 1 to about 10.

Suitable non-ionic surfactants according to the invention include, but are not limited to, ethoxylated fatty alcohol ethers, PEG castor oils, PEG esters, propylene glycol esters, glyceryl esters and derivatives, polymeric ethers, sorbitan derivatives, fatty alcohols, emulsifying waxes, and mixtures thereof.

In an embodiment, the non-ionic surfactant is an ethoxylated fatty alcohol ether. Exemplary ethoxylated fatty alcohol ethers include, but are not limited to, steareth-2, steareth-10, steareth-20, steareth-21, steareth-40, steareth-100, beheneth-10, ceteareth-2, ceteareth-3, ceteareth-5, ceteareth-6, ceteareth-10, ceteareth-12, ceteareth-15, ceteareth-20, ceteareth-21, ceteareth-22, ceteareth-25, ceteareth-30, ceteareth-31, ceteareth-32, ceteareth-33, ceteth-2, ceteth-10, ceteth-20, ceteth-23, choleth-24, isoceteth-20, laureth-2, laureth-3, laureth-4, laureth-5, laureth-9, laureth-10, laureth-12, laureth-15, laureth-20, laureth-21, laureth-22, laureth-23, nonoxynol-9, nonoxynol-15, octoxynol-1, octoxynol-9, oleth-2, oleth-5, oleth-10, oleth-20, C20-40 pareth-24 and trideceth-10, and mixtures thereof.

In an embodiment, the non-ionic surfactant is a PEG castor oil. Exemplary PEG castor oils include, but are not limited to, PEG-7 hydrogenated castor oil, PEG-25 hydrogenated castor oil, PEG-30 castor oil, PEG-33 castor oil, PEG-35 castor oil, PEG-36 castor oil, PEG-40 castor oil, PEG-40 hydrogenated castor oil, PEG-50 castor oil, PEG-54 hydrogenated castor oil, PEG-60 castor oil and PEG-60 hydrogenated castor oil, and mixtures thereof.

In an embodiment, the non-ionic surfactant is a PEG ester. Exemplary PEG esters include, but are not limited to, PEG-4 dilaurate, PEG-150 distearate, PEG-12 glyceryl laurate, PEG-120 glyceryl stearate, PEG-6 isostearate, PEG-4 laurate, PEG-8 laurate, PEG-20 methyl glucose sesquistearate, PEG-5 oleate, PEG-6 oleate, PEG-10 oleate, PEG-25 propylene glycol stearate, PEG-2 stearate, PEG-6 stearate, PEG-6-32 stearate, PEG-8 stearate, PEG-9 stearate, PEG-20 stearate, PEG-40 stearate, PEG-45 stearate, PEG-50 stearate and PEG-100 stearate, and mixtures thereof.

In an embodiment, the non-ionic surfactant is a propylene glycol ester. Exemplary propylene glycol esters include, but are not limited to, propylene glycol laurate, propylene glycol palmitostearate, propylene glycol ricinoleate and propylene glycol stearate, and mixtures thereof.

In an embodiment, the non-ionic surfactant is a glyceryl ester or derivative. Exemplary glyceryl esters and derivatives include, but are not limited to, glyceryl behenate, glyceryl dibehenate, glyceryl dioleate, glyceryl distearate, glyceryl isostearate, glyceryl laurate, glyceryl linoleate, glyceryl monostearate, glyceryl oleate, glyceryl palmitate, glyceryl ricinoleate, glyceryl stearate, PEG-23 glyceryl cocoate, PEG-6 caprylic/capric glycerides, PEG-7 glyceryl cocoate, polyglyceryl-10 diisostearate, polyglyceryl-2 diisostearate, polyglyceryl-3 diisostearate and polyglyceryl-6 diisostearate, PEG-12 glyceryl laurate, PEG-120 glyceryl stearate, and mixtures thereof.

In an embodiment, the non-ionic surfactant is a polymeric ether. Exemplary polymeric ethers include, but are not limited to, poloxamer 124, poloxamer 181, poloxamer 182, poloxamer 184, poloxamer 188, poloxamer 237, poloxamer 331, poloxamer 338 and poloxamer 407, and mixtures thereof.

In an embodiment, the non-ionic surfactant is a sorbitan derivative. Exemplary sorbitan derivatives include, but are not limited to, polysorbate 20, polysorbate 40, polysorbate 60, polysorbate 65, polysorbate 80, sorbitan isostearate, sorbitan monolaurate, sorbitan monooleate, sorbitan monopalmitate, sorbitan monostearate, sorbitan sesquioleate, sorbitan trioleate and sorbitan tristearate, and mixtures thereof.

In an embodiment, the non-ionic surfactant is a fatty alcohol. Exemplary fatty alcohols include, but are not limited to, isostearyl alcohol, caprylyl alcohol, decyl alcohol, lauryl alcohol, myristyl alcohol, behenyl alcohol, lanolin alcohol, arachidyl alcohol, oleyl alcohol, palm alcohol, isocetyl alcohol, cetyl alcohol, stearyl alcohol and cetearyl alcohol, and mixtures thereof. In one embodiment, the fatty alcohol is a mixture of cetyl alcohol and stearyl alcohol, known as cetearyl alcohol (also known as cetostearyl alcohol).

In an embodiment, the non-ionic surfactant is an emulsifying wax, e.g. a non-ionic emulsifying wax also known as emulsifying wax NF, or emulsifying wax BP. In an embodiment, the emulsifying wax is a mixture of cetearyl alcohol and polysorbate 60. In another embodiment, the emulsifying wax is a proprietary blend known as “Polawax NF” ™ (Croda Inc, Edison, N.J., USA).

In one embodiment, the surfactant comprises one or more ethoxylated fatty alcohol ethers. In another embodiment, the ethoxylated fatty alcohol ether is a mixture of steareth-2 and steareth-20.

In one embodiment, the surfactant comprises a mixture of an ethoxylated fatty alcohol ether and a sorbitan derivative. In another embodiment, the mixture of an ethoxylated fatty alcohol ether and a sorbitan derivative is a mixture of steareth-2, steareth-20 and polysorbate 80.

In one embodiment, when there are 2 surfactants present in the formulation, each surfactant is present in an amount from about 0.5% to about 5% by weight, based on the total weight of the composition. In another embodiment, when there are 3 surfactants present in the formulation, each surfactant is present in an amount from about 0.5% to about 5% by weight, based on the total weight of the composition. Similarly, if there are 4 or more surfactants present they are each present in an amount from about 0.5% to about 5% by weight, based on the total weight of the composition.

In one embodiment, the surfactant comprises a mixture of an ethoxylated fatty alcohol ether and an emulsifying wax. In another embodiment, the surfactant comprises a mixture of an ethoxylated fatty alcohol ether, a sorbitan derivative and an emulsifying wax. Suitably, the mixture of an ethoxylated fatty alcohol ether and an emulsifying wax is a mixture of steareth-2, steareth-20, and Polawax™ NF. Suitably, the mixture of an ethoxylated fatty alcohol ether, a sorbitan derivative and an emulsifying wax is a mixture of steareth-2, steareth-20, polysorbate 80 and Polawax™ NF. In an alternative embodiment, the surfactant comprises a mixture of an ethoxylated fatty alcohol ether and a fatty alcohol. Suitably, the mixture of an ethoxylated fatty alcohol ether and a fatty alcohol is a mixture of steareth-2, steareth-20, and cetearyl alcohol.

In another embodiment, the surfactant comprises a mixture of an ethoxylated fatty alcohol ether, a sorbitan derivative and a fatty alcohol. Suitably, the mixture of an ethoxylated fatty alcohol ether, a sorbitan derivative and a fatty alcohol is a mixture of steareth-2, steareth-20, polysorbate 80 and cetearyl alcohol.

### Glidants

In certain embodiments, the pharmaceutical composition further comprises a glidant. In certain embodiments, the glidant may be a combination of glidants. The glidants may be selected from a group consisting of silica, cyclomethicone, magnesium stearate, and any combinations thereof.

Buffers of pH-Adjusting Agents

In certain embodiments, the pharmaceutical composition further comprises a pharmaceutically acceptable buffers or pH adjusting agents. In an embodiment, the pH adjusting agent is an acid, an acid salt, or a mixture thereof. Suitably, the acid is selected from the group consisting of lactic acid, acetic acid, maleic acid, succinic acid, citric acid, benzoic acid, boric acid, sorbic acid, tartaric acid, edetic acid, phosphoric acid, nitric acid, sulphuric acid and hydrochloric acid, and mixtures thereof.

In another embodiment, the pH adjusting agent is a buffer. Suitably, the buffer is selected from the group consisting of citrate/citric acid, acetate/acetic acid, phosphate/phosphoric acid, propionate/propionic acid, lactate/lactic acid, ammonium/ammonia and edetate/edetic acid. In one embodiment, the pH adjusting agent is a buffer which is citrate/citric acid.

Suitably, the pH adjusting agent is present in the composition in an amount from about 0.01% to about 10% by weight, based on the total weight of the composition. In an embodiment, the pH of the composition is adjusted with a pH adjusting agent to a pH of from about 4 to about 7, such as from about 4.5 to about 6.5.

The pharmaceutically acceptable buffer may be selected from phosphoric acid, citric acid, salts thereof, or any combinations thereof. In certain embodiments, the pharmaceutical composition comprises from about 0.01% (w/w) to about 5% (w/w) of a pharmaceutically acceptable buffer.

### Additional Excipients

The pharmaceutical compositions of the invention may further comprise one or more additional dermatologically acceptable excipients. Exemplary additional dermatologically acceptable excipients include, but are not limited to, a chelating agent, a co-solvent, a humectant, a fragrance, a colorant, and mixtures thereof. In certain embodiments, the pharmaceutical composition may further comprise excipients selected from group consisting of: petrolatum, diethyl sebacate, coconut oil, stearyl alcohol, and any combinations thereof.

In an aspect, the invention further provides a preparation of pharmaceutical compositions of the invention. In certain embodiments, the process comprises: mixing a solvent, ALK5 inhibitor, a permeation enhancer, an antioxidant, and a thickening agent to thereby produce a pharmaceutical composition for topical application. In certain embodiments, the solvent comprises an aqueous solvent and a non-aqueous solvent. In certain embodiments, the mixing step involves dissolving hydrophobic excipients in the non-aqueous solvent to form a non-aqueous solution in a specific order. In one embodiment, the aqueous solvent is mixed the non-aqueous solution to form a mixture. In one embodiment, the process further comprises addition of the thickening agent to the mixture in a specific order.

The exemplary embodiments of the invention are provided below. The exemplary embodiments are provided as examples of the invention, and they should not be considered limiting on the scope of the invention. In the exemplary embodiments listed below, the compositions may include any excipients corresponding to the functional roles of the excipients provided below. In particular, the permeation enhancers, solvents, antioxidants, thickening agents, and preservatives could be any of the commonly used excipients for these functions in the pharmaceutical compositions, or any of the excipients described in this application.

### Exemplary Embodiment 1

Exemplary embodiment 1 comprises:
ALK-5 kinase inhibitor: 0.05% (w/w)-5% (w/w);
Permeation enhancer: 10% (w/w)-70% (w/w);
Solvent: 30% (w/w)-98% (w/w);
Antioxidant: 0.05% (w/w)-0.5% (w/w); and
Thickening Agent: 0.5% (w/w)-10% (w/w).

### Exemplary Embodiment 2

Exemplary embodiment 2 comprises:
ALK-5 kinase inhibitor: 0.05% (w/w)-5% (w/w);
Permeation enhancer: 10% (w/w)-70% (w/w);
Solvent: 30% (w/w)-98% (w/w);
Antioxidant: 0.05% (w/w)-0.5% (w/w);
Thickening Agent: 0.5% (w/w)-10% (w/w); and
Preservative: 0.05% (w/w)-0.5% (w/w).

### Exemplary Embodiment 3

Exemplary embodiment 3 comprises:
ALK-5 kinase inhibitor: 0.05% (w/w)-2% (w/w);
Permeation enhancer: 20% (w/w)-60% (w/w);
Solvent: 40% (w/w)-95% (w/w);
Antioxidant: 0.1% (w/w)-0.25% (w/w);
Thickening Agent: 1% (w/w)-5% (w/w); and
Preservative: 0.1% (w/w)-0.3% (w/w).

### Exemplary Embodiment 4

Exemplary embodiment 4 comprises:
ALK-5 kinase inhibitor: 0.05% (w/w)-2% (w/w);
Permeation enhancer: 20% (w/w)-60% (w/w);
Solvent: 40% (w/w)-95% (w/w);
Antioxidant: 0.1% (w/w)-0.25% (w/w); and
Thickening Agent: 1% (w/w)-5% (w/w).

### Exemplary Embodiment 5

Exemplary embodiment 5 comprises:
ALK-5 kinase inhibitor: 0.08% (w/w)-1.5% (w/w);
Permeation enhancer: 30% (w/w)-50% (w/w);
Solvent: 50% (w/w)-95% (w/w);
Antioxidant: 0.1% (w/w)-0.25% (w/w);
Thickening Agent: 1% (w/w)-3% (w/w); and
Preservative: 0.15% (w/w)-0.25% (w/w).

### Exemplary Embodiment 6

Exemplary embodiment 6 comprises:
ALK-5 kinase inhibitor: 0.08% (w/w)-1.5% (w/w);
Permeation enhancer: 30% (w/w)-50% (w/w);
Solvent: 50% (w/w)-95% (w/w);
Antioxidant: 0.1% (w/w)-0.25% (w/w); and
Thickening Agent: 1% (w/w)-3% (w/w).

### Exemplary Embodiment 7

Exemplary embodiment 7 comprises:
ALK-5 kinase inhibitor: 0.3%, 0.5% or 1% (w/w);
Isopropyl myristate: 10%-30% (w/w);
Hexylene glycol: 10%-25% (w/w);
Benzyl alcohol: 0.5%-1.5% (w/w);
Hydroxypropyl methyl cellulose (HPMC): 1%-5% (w/w);
Propyl gallate: 0.1%-0.5%; and
Water: q.s.

### Exemplary Embodiment 8

Exemplary embodiment 8 comprises:
ALK-5 kinase inhibitor: 0.3%, 0.5% or 1% (w/w);
Isopropyl myristate: 25% (w/w);
Hexylene glycol: 20% (w/w);
Benzyl alcohol: 1% (w/w);
Hydroxypropyl methyl cellulose (HPMC): 2.5% (w/w);
Propyl gallate: 0.3%; and
Water: q.s.

### Exemplary Embodiment 9

Exemplary embodiment 9 comprises:
ALK-5 kinase inhibitor: 0.3%, 0.5% or 1% (w/w);
Diethylene glycol monoethyl ether: 20%-60% (w/w);
Ethyl alcohol: 10%-25% (w/w);
Polyethylene Glycol: 2.5%-10% (w/w);
Methylparaben/Propylparaben (or combination thereof): 0.1%-0.5% (w/w);
Butylated Hydroxytoluene: 0.1-0.5% (w/w);
Hydroxypropyl cellulose (HPC): 1%-5% (w/w); and
Water: q.s.

### Exemplary Embodiment 10

Exemplary embodiment 10 comprises:
ALK-5 kinase inhibitor: 0.3%, 0.5% or 1% (w/w);
Diethylene glycol monoethyl ether: about 45% (w/w);
Ethyl alcohol: about 15% (w/w);
Polyethylene Glycol: 5% (w/w);
Methylparaben/Propylparaben (or combination thereof): about 0.25% (w/w);
Butylated Hydroxytoluene: 0.2% (w/w);
Hydroxypropyl cellulose (HPC): 2% (w/w); and
Water: q.s.

### Exemplary Embodiment 11

Exemplary embodiment 11 comprises:
ALK-5 kinase inhibitor: 0.3%, 0.5% or 1% (w/w);
Lauric Acid: 20%-60% (w/w);
Hexyl alcohol: 10%-25% (w/w);
Polyethylene Glycol: 2.5%-10% (w/w);
Benzalkonium chloride: 0.1%-0.5% (w/w);
Tocopherol: 0.1-0.5% (w/w);
Sodium carboxymethylcellulose: 1%-5% (w/w); and
Water: q.s.

### Exemplary Embodiment 12

Exemplary embodiment 12 comprises:
ALK-5 kinase inhibitor: 0.3%, 0.5% or 1% (w/w);
Laurie Acid: about 45% (w/w);
Hexyl alcohol: about 15% (w/w);
Polyethylene Glycol: about 5% (w/w);
Benzalkonium chloride: about 0.2% (w/w);
Tocopherol: about 0.2% (w/w);
Sodium carboxymethylcellulose: about 2% (w/w); and
Water: q.s.

### Exemplary Embodiment 13

Exemplary embodiment 13 comprises:
ALK-5 kinase inhibitor: 0.1% or 1% (w/w);
Diethylene glycol monoethyl ether: 46% (w/w);
Alcohol 190 Proof, USP: 16% (w/w);
Propylene Glycol, USP: 5% (w/w);
Hydroxypropyl cellulose: 2% (w/w);
Methylparaben: 0.2% (w/w);
Propylparaben: 0.02% (w/w);
Butylated Hydroxytoluene: 0.2% (w/w); and
Sterile water: q.s.

### Exemplary Embodiment 14

Exemplary embodiment 14 comprises:
ALK-5 kinase inhibitor: 0.3% or 1% (w/w);
Diethylene glycol monoethyl ether: 46% (w/w);
Cyclomethicone, NF: 5% (w/w);
Propylene Glycol, USP: 5% (w/w);
Methylparaben: 0.2% (w/w);
Propylparaben: 0.02% (w/w);
Polysorbate 80, NF: 2% (w/w);
Carbopol 980, NF: 0.4% (w/w);
Pemulen TR-1: 0.4% (w/w);
Petrolatum, USP: 13% (w/w)
Arlacel 165: 6% (w/w)
Trolamine: q.s. for pH to 5.5-6.0; and
Sterile water: q.s.

### Exemplary Embodiment 15

The exemplary embodiment 15 comprises:
ALK-5 kinase inhibitor: 0.3% or 1% (w/w);
PEG-35 Castor Oil 1.9% (w/w);
PEG-60 Hydrogenated Castor Oil 1.9% (w/w);
Propylene glycol monolaurate (type II), NF: 3% (w/w);
Caprylic/capric triglyceride, NF: 15% (w/w);
2-Phenoxyethanol: 1% (w/w);
Cyclomethicone, NF: 2.0% (w/w);
Diethyl sebacate: 10% (w/w);
Coconut Oil: 10% (w/w);
Butylated Hydroxytoluene, NF: 0.1% (w/w);
Stearyl Alcohol, NF: 5% (w/w);
Glyceryl Stearate and PEG-75 Stearate: 7% (w/w);
Dibasic sodium phosphate, dried, USP: 0.35% (w/w);
Anhydrous citric acid, USP: 0.1% (w/w); and
Water: q.s.

### Exemplary Embodiment 16

The exemplary embodiment 16 comprises:
ALK-5 kinase inhibitor: 0.3% or 1% (w/w);
Propylene Glycol: 30% (w/w);
PEG 400 NF: 30% (w/w);
Ethanol USP: 26.5% (w/w);
Benzyl Alcohol: 2.0 (w/w);
Klucel (HPMC): 0.5% (w/w);
Hydroxypropyl cellulose: 0.5% (w/w); and
Water: q.s.

### Exemplary Embodiment 17

The exemplary embodiment 17 comprises:
ALK-5 kinase inhibitor: 0.3% or 1% (w/w);
Propylene Glycol: 9.9% (w/w);
Ethanol USP: 79.2% (w/w);
Di-isopropyl adipate: 9.9% (w/w); and
Water: q.s.

### Exemplary Embodiment 18

The exemplary embodiment 18 comprises:
ALK-5 kinase inhibitor: 0.3% or 1% (w/w);
Propylene Glycol: 20% (w/w);
Ethanol, USP: 20% (w/w);
Transcutol P: 30% (w/w);
Brij-L4-LQ: 4.9% (w/w);
Isopropyl myristate: 14% (w/w);
Isopropyl palmitate: 7% (w/w);
Cycolmethicone: 1% (w/w);
BHT: 0.1% (w/w); and
Hydroxypropyl cellulose: 2% (w/w).

### Exemplary Embodiment 19

The exemplary embodiment 19 comprises:
ALK-5 kinase inhibitor: 0.3% or 1% (w/w);
Propylene Glycol: 30% (w/w);
Transcutol P: 24% (w/w);
Brij-L4-LQ: 4.9% (w/w);
Isopropyl myristate: 14% (w/w);
Isopropyl palmitate: 7% (w/w);
Glycerin NF: 5% (w/w);
Gelot-64: 4% (w/w);
Arlacel 165: 6.9% (w/w);
Glyceryl isostearate: 1.1% (w/w); and
BHT: 0.1% (w/w).

### Exemplary Embodiment 20

The exemplary embodiment 20 comprises:
ALK-5 kinase inhibitor: 1%, 2% or 3% (w/w);
Ceteth-2: 2% (w/w);
Ceteth-20: 2% (w/w);
Propylene glycol monolaurate (type II): 3% (w/w);
Caprylic/capric triglyceride: 15% (w/w);
Isostearic acid: 21% (w/w);
Benzyl alcohol: 2.7% (w/w);
ST-Cyclomethicone-5 NF: 2% (w/w);
BHT: 0.1% (w/w);
Stearyl alcohol: 2% (w/w) or 6% (w/w);
Mono and diglycerides and polyoxyl stearate: 8% (w/w) or 4% (w/w); and
Water: q.s.

### Exemplary Embodiment 21

The exemplary embodiment 21 comprises:
ALK-5 kinase inhibitor: 0.3% or 1% (w/w);
Propylene Glycol: 30% (w/w);
Transcutol P: 24% (w/w);
Brij-L4-LQ: 4.9% (w/w);
Isopropyl myristate: 14% (w/w);
Isopropyl palmitate: 7% (w/w);
Glycerin NF: 5% (w/w);
Gelot-64: 4% (w/w);
Arlacel 165: 6.9% (w/w);
Glyceryl isostearate: 1.1% (w/w);
Citrate buffer: about 0.5% (q.s. pH 5.5-6.5); and
BHT: 0.1% (w/w).

### Exemplary Embodiment 22

Exemplary embodiment 22 comprises:
ALK-5 kinase inhibitor: 0.3% or 1% (w/w);
Dimethyl sulfoxide: 5%-25% (w/w);
Isopropyl alcohol: 10%-25% (w/w);
Polyethylene Glycol: 2.5%-10% (w/w);
Methylparaben/Propylparaben (or combination thereof): 0.1%-0.5% (w/w);
Benzyl alcohol: 0.1-0.5% (w/w);
Hydroxypropyl cellulose (HPC): 1%-5% (w/w); and
Water: q.s.

### Dosing

In certain embodiments, the total dose of the ALK-5 kinase inhibitor, such as for example, the total dose of Compound 1, applied to the skin of the patient would be from about 5 mg-40 mg or 0.5 g-4 g of a 1% w/w pharmaceutical composition. In certain embodiments, the total dose of the ALK-5 kinase inhibitor applied to the skin of the patient would be from about 10-30 mg. In certain embodiments, the total dose of ALK-5 kinase inhibitor applied to the skin of the patient would be from about 7.5-20 mg. In certain embodiments, the total dose of the ALK-5 kinase inhibitor applied to the skin of the patient would be from about 10-20 mg.

In certain embodiments, from about 100 mg to about 5 g of the pharmaceutical composition is applied to the skin of the patient. In certain embodiments, from about 200 mg to about 2.5 g of the pharmaceutical composition is applied to the skin of the patient. In certain embodiments, from about 500 mg to about 2 g of the pharmaceutical composition is applied to the skin of the patient. In certain embodiments, from about 1 g to about 2 g of the pharmaceutical composition is applied to the skin of the patient.

In certain embodiments, the pharmaceutical composition is applied to the entire affected area of the skin of the patient suffering from a dermatological condition. In certain embodiments, the pharmaceutical composition is applied to from about 10 cm² to about 400 cm² of the affected body surface area. In certain embodiments, the pharmaceutical composition is applied to from about 50 cm² to about 300 cm² of the affected body surface area. In certain embodiments, the pharmaceutical composition is applied to about 200 cm² of the affected body surface area.

In certain aspects, the pharmaceutical composition of the invention is applied about once daily. In certain embodiments, the pharmaceutical composition of the invention is applied about twice daily.

### EXAMPLES

The examples below describe various embodiments of the pharmaceutical compositions of the invention.

In conducting analysis of formulations, following type of formulations were prepared:
Non-Aqueous solutions—Regular (e.g High Flux formulation);
Non-Aqueous, Film-Forming solutions;
Ointments (non-aqueous);
Non-Aqueous Gels;
Aqueous Gels;
Aqueous Creams;
Aqueous Lotions.

As explained in the Examples below, the formulations of the invention were designed to have a higher dermal deposition, epidermal deposition, increase in flux over a period of time, and dermal concentration of Compound 1. In certain embodiments, the pharmaceutical composition of the invention have a higher concentration of Compound 1 in the dermis than the corresponding IC₅₀ value of the Compound 1.

In some embodiments, the pharmaceutical compositions of the invention may have from about 3-fold to about 9-fold increase in the dermal deposition of Compound 1 as compared to the comparative formulations. In certain embodiments, the pharmaceutical compositions of the invention may have 1-1.50 fold increase in the epidermal deposition of Compound 1 as compared to the comparative formulations. In some embodiments, the pharmaceutical compositions of the invention may have from about 2-fold to about 6-fold increase in the flux of delivered dose of Compound 1 over a period of 24 hours as compared to a comparative formulation. In certain embodiments, the pharmaceutical compositions of the invention may have from about 20-fold to about 150-fold higher dermal concentration of Compound 1 as compared to the IC₅₀ value of Compound 1.

In certain embodiments, the compositions of the invention are topical creams. As evident from the data provided in the Examples below, the topical creams typically have from about 1% to about 2% of Compound 1. The concentration of about 1% to about 2% of Compound 1 results in the target concentration of Compound 1 in the skin. In certain embodiments, the creams of the invention further comprise surfactants and/or oils. The creams of the invention have a lower dermal penetration rate as compared to the gels, but they have high dermal deposition in the in vitro permeation testing (IVPT) evaluations.

In certain embodiments, the compositions of the invention are topical gels. The gels may be both aqueous or non-aqueous gels. In certain embodiments, the gels of the invention also comprise from about 1% to about 2% of Compound 1.

### Example 1

### Solubility of Compound 1 in Different Solvents:

Tables 2 and 3 provide the solubility of the exemplary ALK-5 kinase inhibitor Compound 1 in various solvents.

TABLE 2
Solubility of Compound 1

| | | |
|---|---|---|
| | | Solubility |
| # | Solvent | (mg/mL) |

Aqueous Systems

| | | |
|---|---|---|
| 1 | Water | 0.02 |
| 2 | Sod.Citrate buffer (pH3) | 2.19 |
| 3 | Sod.Citrate buffer (pH4) | 0.29 |
| 4 | Sod.Citrate buffer (pH5) | 0.07 |
| 5 | Phosphate Buffer, pH 6.5 | 0.02 |

Water Miscible solvents

| | | |
|---|---|---|
| 6 | Ethanol | 30.8 |
| 7 | Propylene Glycol (PG) | 23.7 |
| 8 | PEG 400 | 43.9 |
| 9 | Acetone | 38.3 |
| 10 | DMSO | 83.7 |
| 11 | Hexylene Glycol | 50.2 |
| 12 | Transcutol (DEGEE) | 115.1 |
| 13 | Propylene Carbonate | 24.1 |

Water Immiscible Solvents

| | | |
|---|---|---|
| 14 | Diisopropyl Adipate | 50.5 |
| 15 | Phenethyl Alcohol | >300 |
| 16 | Benzyl Alcohol | >300 |

TABLE 3
Solubility of Compound 1

| | | Solubility |
|---|---|---|
| # | Solvent | (mg/mL) |
| 17 | Isopropyl Myristate | 3.45 |
| 19 | Mineral Oil | 0.54 |
| 21 | Octyldodecanol | 5.37 |
| 22 | PPG-15 Stearyl Ether | 4.87 |
| 23 | Medium Chain Triglycerides | 5.31 |
| 24 | Cycolmethicone NF | 0.19 |
| 25 | Diethyl Sebacate | 13.06 |
| 26 | Dimethicone 100 CST | 0.11 |

### Example 2

### Formulations 1 and 2:

Table 4 provides examples of pharmaceutical compositions comprising Compound 1. Formulation 1 is an aqueous gel, which may be used for early clinical evaluation of the performance of Compound 1. Formulation 2 is a high-flux vehicle intended for the evaluation of topical safety and providing both high skin deposition and high flux/exposure in the systemic circulation.

TABLE 4
Formulations 1 and 2:

| | Formulation 1: Gel | Formulation 2: High Flux |
|---|---|---|
| | Formulation | Vehicle (HFV) |
| | (% w/w) | Formulation |
| Ingredient | (“PFE”) | (“PFE2”) (% w/w) |
| Compound 1 | 0 to 2.0 | 0 to2.0 |
| Propylene Glycol NF | 30.0 | 10.0 |
| PEG 400 NF | 30.0 | |
| Ethanol USP | 26.5 | 78 to 80 |
| Di-Isopropyl Adipate | | 10.0 |
| Benzyl Alcohol USP/NF | 2.0 | |
| Klucel ( HPMC) | 0.5 | |
| Water | 9 to 11.0 | |
| TOTAL | 100.0% | 100.0% |

There was a 30-35 fold increase in dermal penetration rate via IVPT of Formulation 2 over that of Formulation 1 at the 1% w/w loading level of Compound 1. However, Formulation 2 demonstrated some topical reactions in a limited safety evaluation in Minipigs when administered at the 1% level and a dose of 0.5 mg/cm². No similar reaction was observed for Formulation 1. This indicates that topical reaction may be driven by both high flux into the dermal tissues and the aggressive nature of solvent formulation used.

In conducting the experiments, the inventors found that Compound 1 interacts with certain pharmaceutical excipients included in the formulations. These reactions may be responsible for strong inflammatory responses when the sample is applied on the skin. In particular, inflammatory responses were particularly high with formulations comprising a high concentration of non-aqueous solvents. In particular, pharmaceutical compositions with a high concentration of ethanol and/or isopropyl adipate caused a significant inflammatory response.

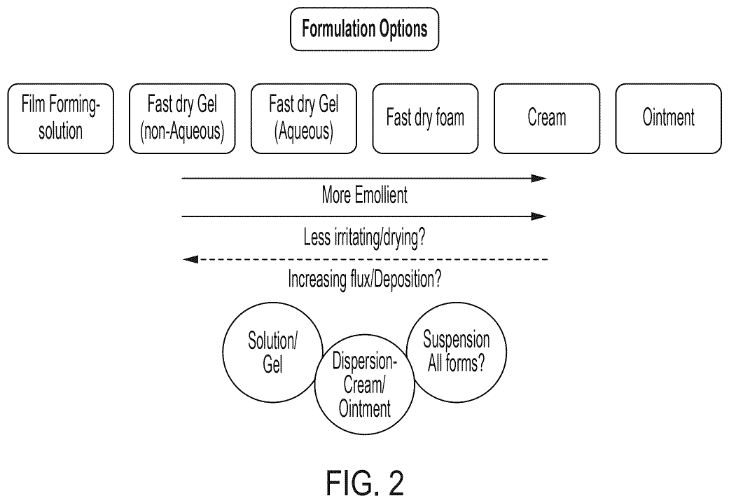

Thus, the formulation development should balance several factors, including balancing the dermal flux/permeation with inflammation potential. FIG. 2 demonstrates the various factors to be evaluated in the development of the formulations.

### Example 3

Solubility assessment in various excipients.

Table 5 provides the solubility of Compound 1 in various solvents and excipient systems to analyze the topical systems to be utilized with the formulations. Compound 1 in excess of its visual solubility was added to known weight of solvent system and stirred for a minimum of 24 hours at 25° C. prior to centrifugation and HPLC analysis of supernatant. The solubility in formulated systems is summarized in Table 5.

TABLE 5
Solubility of Compound 1 in the Excipients

| | Range of Aqueous | Max solubility of |
|---|---|---|
| Formulation Type | component (% w/w) | Compound 1 (%/w) |
| 1. Non-Aqueous Gels & | 0.0% | 3-3.5% |
| Film forming | | |
| solutions | | |
| 2. Aqueous Gels | 10-30% | 1-2.7% |
| 3. Creams & Lotions | 30-40% | 1-1.5% |

The quantity of Compound 1 that can be included in the formulation is dependent on the excipients and their ability to complex with Compound 1.

### Example 4

### Excipient Compatibility Studies:

Stability studies were conducted to evaluate compatibility and stability of Compound 1 with some of the excipients included in the solubility experiments. The stability was assessed using the validated API HPLC method after storage at room temperature (RT), 40° C. and 50° C. for 2 weeks. The summary of the data generated from these experiments is provided below in Table 6 below.

TABLE 6
Stability results when stored at RT, 40 and 50° C. for 2 weeks

| | RT | | | |
|---|---|---|---|---|
| Solvent | storage | 40° C. | 50° C. | Conclusion |
| pH 3 Citrate | No Loss | No Loss | No Loss | STABLE AT ALL |
| buffer | | | | CONDITIONS |
| pH4 Citrate | No Loss | No Loss | No Loss | STABLE AT ALL |
| buffer | | | | CONDITIONS |
| pH5 Citrate | No Loss | No Loss | No Loss | STABLE AT ALL |
| buffer | | | | CONDITIONS |
| PEG 400 | No Loss | 10% loss | ~28% loss | Unstable at > RT |
| PEG 400 + | No Loss | No Loss | No Loss | Stabilized by |
| 0.1% BHT | | | | antioxidant |
| | | | | BHT |
| Transcutol | No Loss | No Loss | No Loss | STABLE AT ALL |
| (DEGEE) | | | | CONDITIONS |
| Transcutol | No Loss | No Loss | No Loss | STABLE AT ALL |
| (DEGEE) | | | | CONDITIONS |
| +0.1% BHT | | | | |
| Propylene | No Loss | No Loss | No Loss | STABLE AT ALL |
| Glycol | | | | CONDITIONS |
| Benzyl Alcohol | No Loss | No Loss | No Loss | STABLE AT ALL |
| | | | | CONDITIONS |
| Benzyl Alcohol | No Loss | No Loss | No Loss | STABLE AT ALL |
| +0.1% BHT | | | | CONDITIONS |
| Phenoxyethanol | No Loss | No Loss | No Loss | STABLE AT ALL |
| | | | | CONDITIONS |
| Phenoxyethanol | No Loss | No Loss | No Loss | STABLE AT ALL |
| +0.1% BHT | | | | CONDITIONS |
| Diethyl | No Loss | 3-4% loss | 6% loss | Some Instability |
| Sebacate | | | | indicated of unknown |
| | | | | mechanism |

As evident from the data above, the solubility and compatibility experiments demonstrate that Compound 1 is relatively stable except with PEG400 and Diethyl Sebacate even though they offer good solubility benefits to the molecule. PEG400 instability was overcome with the use of 0.1% BHT, an antioxidant.

The source of instability in diethyl sebacate is unknown.

### Example 5

The Example provides Formulations 3, 4, 5, and 6, which is disclosed in Table 7: Table 7:

TABLE 7
Formulations:

| | For- | For- | For- | Formulation |
|---|---|---|---|---|
| | mulation | mulation | mulation | 6 (%ww) |
| | 3 (% ww) | 4 (% ww) | 5 (%ww) | (F142 |
| | (PFE 1 | (HFV-PFE | (F8-Non- | Aqueous |
| Ingredient | Gel) | Soln.) | aqeous Gel) | Gel) |
| Compound 1 | 1.0 | 1.0 | 2.0 | 2.0 |
| Propylene Glycol | 30.0 | 9.9 | 20.0 | 10.0 |
| NF | | | | |
| PEG 400 NF | 30.0 | | | |
| Ethanol USP | 26.5 | 79.2 | 19.0 | 39.0 |
| Di-Isopropyl | | 9.9 | | |
| Adipate | | | | |
| Transcutol P | | | 30.0 | 19.3 |
| Laureth-4 (Brij-L4) | | | 4.9 | 4.9 |
| Isopropyl Myristate | | | 14.0 | 3.5 |
| Isopropyl Palmitate | | | 7.0 | 3.5 |
| Caprylic/Capric | | | | 3.0 |
| Triglycerides | | | | |
| (GTCC) | | | | |
| ST-Cyclomethicone- | | | 1.0 | 2.0 |
| 5 NF | | | | |
| BHT | | | 0.1 | 0.1 |
| Benzyl Alcohol | 2.0 | | | 2.7 |
| Klucel (HPMC) | 0.5 | | | |
| HPC HY119 | | | 2.0 | 3.0 |
| Water | 10.0 | | | 7.0 |
| TOTAL | 100.0 | 100.0 | 100.0 | 100.0 |

### Example 6

The Example provides Formulations 7, 8, 9, and 10, which are disclosed in Table 8:

TABLE 8
Formulations 7, 8, 9, and 10

| | Formulation | Formulation | Formulation | Formulation |
|---|---|---|---|---|
| | 7 | 8 | 9 | 10 |
| | Compound 1 | FBM F154 | FBM F156 | FBM F158 |
| Ingredients | (Cream) | (Cream) | (Cream) | (Cream) |
| Compound 1 | 3.00 | 3.00 | 2.00 | 2.00 |
| Ceteth-2 | 2.00 | 2.00 | 2.00 | 2.00 |
| Ceteth-20 | 6.00 | 6.00 | 6.00 | 6.00 |
| Propylene glycol | 3.00 | 3.00 | 3.00 | 3.00 |
| monolaurate | | | | |
| (type II) | | | | |
| Caprylic/capric | 15.00 | 15.00 | 15.00 | 15.00 |
| triglyceride | | | | |
| Isostearic acid | 21.00 | 21.00 | 21.00 | 21.00 |
| Benzyl alcohol | 2.70 | 2.70 | 2.70 | 2.70 |
| ST- | 2.00 | 2.00 | 2.00 | 2.00 |
| Cyclomethicone- | | | | |
| 5NF | | | | |
| BHT | 0.1 | 0.1 | 0.1 | 0.1 |
| Stearyl alcohol | 4.00 | 4.00 | 2.00 | 6.00 |
| Mono and | 6.00 | 4.00 | 8.00 | 4.00 |
| diglycerides and | | | | |
| polyoxyl | | | | |
| stearate(Gelot | | | | |
| 64) | | | | |
| Water | 35.20 | 37.20 | 36.20 | 36.20 |
| Total | 100.00 | 100.00 | 100.00 | 100.00 |

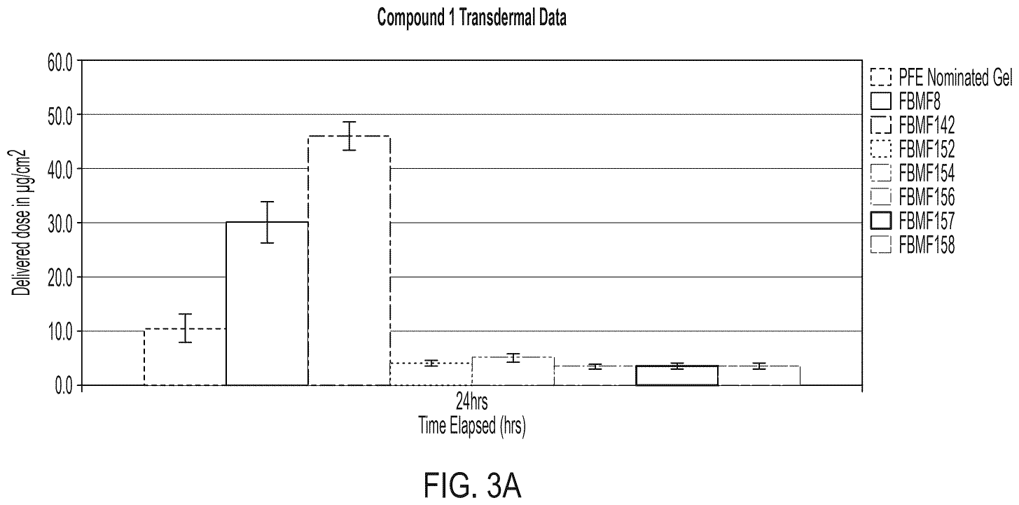

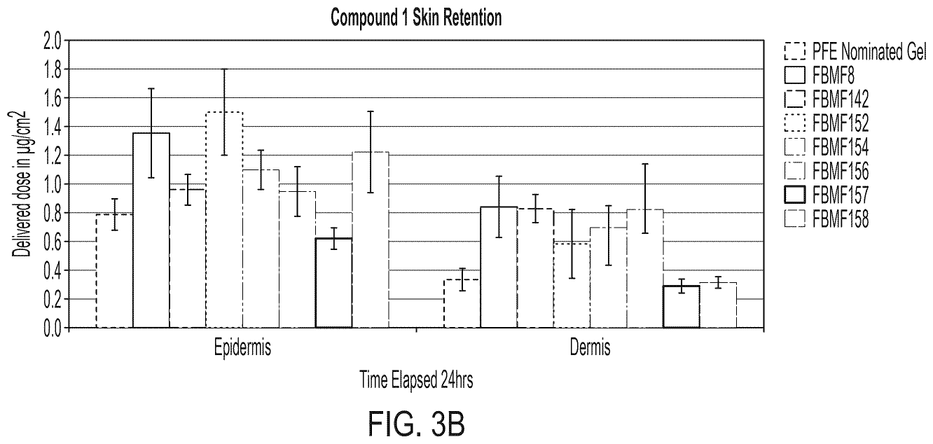

As disclosed in FIGS. 3A and 3B, Gel Formulations 5 and 6 have high in vitro flux rates and dermal deposition, whereas the Cream Formulations 7-10 have low- to -moderate in vitro flux rates but varying dermal deposition rates. Formulation 9 has a high dermal deposition rate whereas Formulation 10 has one of the lowest despite little difference in composition. The permeation data from the skin samples demonstrates that gel formulations have higher flux rates when compared to those of the cream formulations. The data also demonstrates that the ratio of dermal deposition rate to the Flux rate is much higher for cream formulations than for Gels as measured over 24 hours for compound 1. In general, creams have a slower flux and higher depot delivery systems than gels which often contain large amounts of volatile solvents and enhancers that are designed to deliver more rapidly. This appears to be confirmed from the in vitro data. However, in vivo delivery assessment may be a more realistic evaluation due to the extended dosing period available to reach steady state and having both skin metabolism and physiology and blood/lymph circulation intact and functioning.

### Example 7

Example 7 provides Formulations 11, 12, and 13, listed in Table 9 below. The example also provides details of the in vivo permeation studies conducted in pigs.

TABLE 9
Formulations 11, 12, and 13

| | Composition % (w/w) | | | | | |
|---|---|---|---|---|---|---|
| Ingredient | FBM-DDL | FBM-DDL | FBM F245 | FBM F245 | FBM-DDL | |
| Formulation | HPC gel | Cream | Cream | Cream | Carbopol gel | |
| code | (Formn 11) | (Formn 12) | (Formn 13) | (Formn 14) | (Formn 15) | Function |
| Description | GEL | CREAM | CREAM | CREAM | GEL | |
| Compound 1 | 0.3- | 0.3- | 0.3- | 1.00% w/w | 0-1.00% w/w | Active |
| | 1.0% w/w | 1.0% w/w | 1.0% w/w | | | |
| Diethylene | 46.00 | 46.00 | | | 46.00 | Primary solvent |
| Glycol | | | | | | and enhancer |
| Monoethyl | | | | | | |
| Ether NF | | | | | | |
| Alcohol 190 | 16.00 | | | | 16.00 | Solvent and |
| Proof USP | | | | | | enhancer |
| Propylene | 5.00 | 5.00 | | | 5.00 | Enhancer |
| Glycol USP | | | | | | |
| Cyclomethic | | 1.00 | 2.00 | 2.00 | | Glidant |
| one NF | | | | | | |
| Methylparaben | 0.20 | 0.20 | | | 0.2 | Antimicrobial |
| NF | | | | | | preservative |
| Propylparaben | 0.02 | 0.02 | | | 0.02 | Antimicrobial |
| NF | | | | | | preservative |
| BHT NF | 0.20 | 0.20 | 0.10 | 0.1 | 0.2 | Antioxidant |
| Hydroxypropyl- | 2.00 | 2.00 | | | | Thickening agent |
| cellulose | | | | | | |
| Polysorbate | | 2.00 | | | | Surfactant/Emulsifier |
| 80 NF | | | | | | |
| Carbopol | | 0.40 | | | 0.50 | Thickening agent |
| 980 NF | | | | | | |
| Pemulen | | 0.40 | | | | Surfactant/Emulsifier |
| TR-1 | | | | | | |
| Petrolatum | | 13.00 | | | | Oil Phase |
| USP | | | | | | |
| Arlacel 165 | | 6.00 | | | | Surfactant/Emulsifier |
| 50% | | Adj to | | | 3.00 | Viscosity former |
| Trolamine | | pH 5.5-6.0 | | | | |
| solution | | | | | | |
| PEG-35 | | | 1.90 | | | Surfactant/Emulsifier |
| Castor Oil | | | | | | |
| PEG-60 | | | 1.90 | 1.90 | | Surfactant/Emulsifier |
| Hydrogenated | | | | | | |
| Castor Oil | | | | | | |
| Kolliphore | | | | 1.90 | | Surfactant/Emulsifier |
| EL | | | | | | |
| Propylene | | | 3.00 | 3.00 | | Surfactant/enhancer |
| Glycol | | | | | | |
| Monolaurate | | | | | | |
| (type II) NF | | | | | | |
| Caprylic/ | | | 15.00 | 15.00 | | Surfactant/ |
| Capric | | | | | | enhancer |
| Triglycerides | | | | | | |
| NF | | | | | | |
| Isostearic | | | | 5.00 | | Solubilizing lipid |
| Acid | | | | | | |
| 2- | | | 1.00 | 1.00 | | Antimicrobial |
| PhenoxyethanoI | | | | | | preservative |
| Diehtyl | | | 10.00 | | | Oil Phase |
| Sebacate | | | | | | |
| Coconut Oil | | | 10.00 | 5.00 | | Oil Phase |
| Stearyl | | | 5.00 | 5.00 | | Structural Lipid |
| Alcohol NF | | | | | | |
| Ceraphyl 41 | | | | 10.00 | | Surfactant/Emulsifier |
| Glyceryl | | | 7.00 | 7.00 | | Surfactant/Emulsifier |
| Stearate & | | | | | | |
| PEG-75 | | | | | | |
| Stearate | | | | | | |
| Di-basic Sod. | | | 0.35 | | | Buffer |
| Phosphate | | | | | | |
| dried USP | | | | | | |
| Anhydrous | | | 0.10 | | | Buffer |
| Citric Acid | | | | | | |
| USP | | | | | | |
| Sterile | 29.58- | QS to 100% | QS to | QS to | 28.08- | Aqueous Phase |
| Water for | 30.28% w/w | w/w | 100% w/w | 100% w/w | 29.08% w/w | |
| Irrigation | | (~23% | (~41% | (~42% w/w) | | |
| USP | | water) | water) | | | |

### Dermal Tolerability Study:

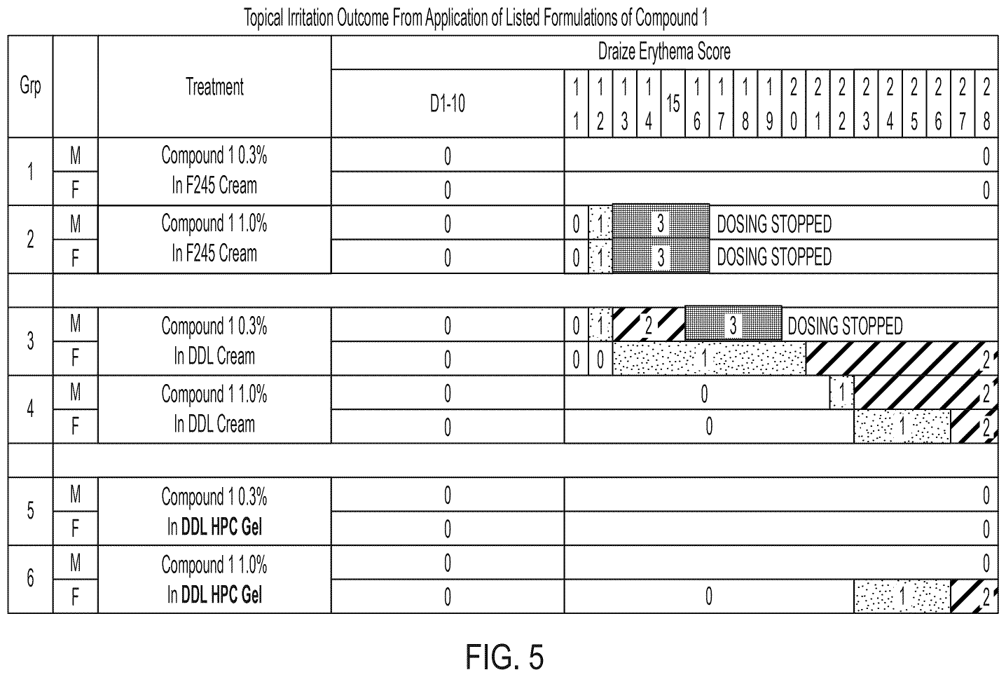

The results from the dermal stability study of these formulations over a period of 28 days are provided in FIG. 5. In particular, FIG. 5 provides Draize scoring of Erythema. The scores indicate: 0—No erythema, 1—very slight erythema (barely perceptible), 2—well-defined erythema, 3—moderate-to-severe erythema, 4—severe erythema (beet redness) to slight eschar formation (injuries in depth).

### In Vivo Performance Evaluation:

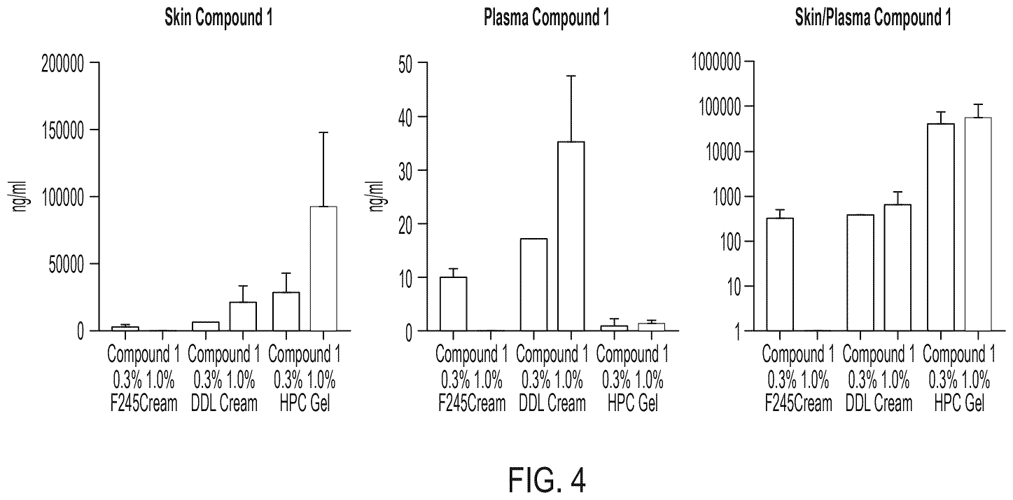

Using N=2 Minipigs per group, a dose ranging, non-GLP study was preformed to evaluate the PK characteristics and toleration (using the traditional Draize scoring system of Erythema) of the three formulations at 0.3 and 1.0% w/w strengths as listed in Table 10 below. The application rate of each was 0.5 mL/kg twice per day (or 1 mL/kg/day) over 10% body surface area (BSA) of the Minipig for 28 days. On the final day, full PK analysis was performed after which the animals were sacrificed to perform the required toxicological parameter evaluations as well as determining skin concentrations of Compound 1 at the final plasma level measured. The results of the IVPT and the PK evaluation is given in Table 10. The In-Vivo data is summarized in FIG. 4.

TABLE 10
In Vitro Permeation Testing over 24 hours compared to In - Vivo PK and toleration 28-day
study data for three COMPOUND 1 formulations all tested at 0.3 and 1.0% w/w strengths

| | In-Vitro Permeation Testing (IVPT)⁴ | | | | In-Vivo Testing in Mini-Pigs¹ | | | | |
|---|---|---|---|---|---|---|---|---|---|
| | | | | Derm. | | | Derm. | | |
| | | | | Selectivity | Mean | Mean | Selectivity | Dermal | |
| | | Epidermis | Dermis | (Mean | Skin | Plasma | (Mean | level vs | |
| | Flux | depot | depot | Dermis/ | Concn. | Concn. | Skin/Plasma | IC₅₀ | |
| Formulation | μg/cm² | (μg/cm²) | (μg/cm²) | Flu × ratio) | (ng/mL) | (ng/mL) | ratio) | (30 ng/ml) | |
| FBM DDL | 2.36 ± | 0.43 ± | 1.23 ± | 0.52 | 29478 | 1.1 | 43,393 | ~1000 | X |
| HPC Gel | 0.27 | 0.09 | 0.3 | | | | | | |
| 0.3% w/w | | | | | | | | | |
| FBM DDL | 7.07 ± | 3.25 ± | 2.17 ± | 0.31 | 93241 | 1.72 | 60,719 | ~3000 | X |
| HPC Gel | 1.53 | 1.03 | 0.23 | | | | | | |
| 1.0% w/w | | | | | | | | | |
| FBM DDL | 2.05 ± | 0.53 ± | 1.05 ± | 0.51 | 7135² | 17.5² | 408² | ~250 | X² |
| Cream | 0.36 | 0.02 | 0.17 | | | | | | |
| 0.3% w/w | | | | | | | | | |
| FBM DDL | 6.23 ± | 0.81 ± | 2.13 ± | 0.34 | 21716 | 35.5 | 709 | ~750 | X |
| Cream | 1.75 | 0.19 | 0.43 | | | | | | |
| 1.0% w/w | | | | | | | | | |
| FBM F245 | 2.43 ± | 0.31 ± | 0.71 ± | 0.29 | 3310 | 10.2 | 337 | ~100 | X |

| | | | | | | | | |
|---|---|---|---|---|---|---|---|---|
| Cream | 0.15 | 0.05 | 0.09 | | | | | |
| 0.3% w/w | | | | | | | | |
| FBM F245 | 3.67 ± | 0.73 ± | 1.60 ± | 0.44 | Not | Not | Not | Not |
| Cream | 0.2 | 0.18 | 0.18 | | measured³ | measured³ | measured³ | measured³ |
| 1.0% w/w | | | | | | | | |

¹28-Day study in N = 2 Minipigs(M/F) dosed at 0.5 mL/kg BID over 10% BSA; PK profile and Skin concentrations determined on Day 28;
²Represents only one Minipig(Female) as dosing stopped(Male) at D20;
³Dosing stopped at D17;
⁴IVPT study run for 24 hours as the viability of skin decreases beyond this point.

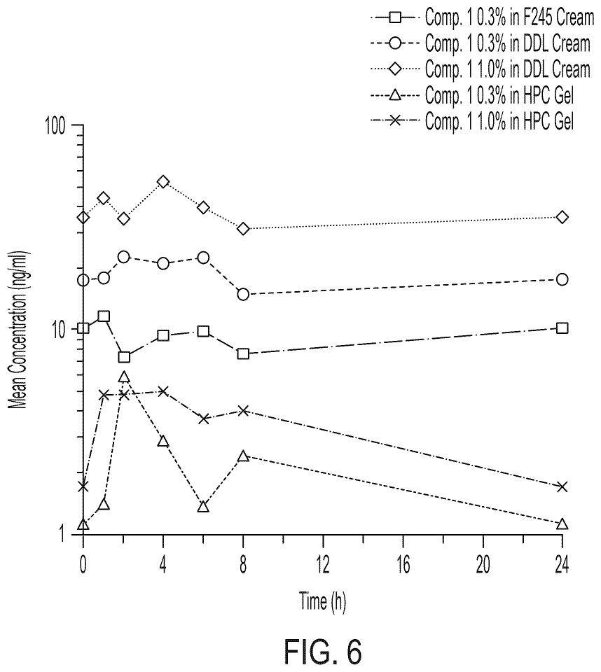

FIG. 6 provides the pharmacokinetic profiles of several embodiments of the invention after administration of the compositions for 28 days.

TABLE 11
Mean dermal concentration with the different formulations,
measured 16 hours after the last dose.
Group Mean Dermal Skin Concentrations (ng/ml) Following
Topical Administration of Formulations

| | Compound 1 |
|---|---|
| Treatment | Dermal Conc. |
| 0.3% in F245 Cream | 3310 |
| (Formulation 13) | |
| 0.3% in DDL Cream | 7135 |
| (Formulation 12) | |
| 1.0% in DDL Cream | 21716 |
| (Formulation 12) | |
| 0.3% in DDL HPC Gel | 29478** |
| (Formulation 11) | |
| 1.0% in DDL HPC Gel | 93241** |
| (Formulation 11) | |

**indicates that formulations have dermal concentrations >3000 fold the IC50 of Compound 1.

TABLE 12
Plasma pharmacokinetic parameters at Day 28:
Group Mean Plasma TK Parameters

| | Cₘₐₓ | AUC₀₋₂₄ |
|---|---|---|
| Treatment | (ng/ml) | (h*ng/ml) |
| Compound 1 0.3% | 11.6 | 221 |
| in F245 Cream | | |
| (Formulation 13) | | |
| Compound 1 0.3% | 22.7 | 422 |
| in DDL Cream | | |
| (Formulation 12) | | |
| Compound 1 1.0% | 52.9 | 887 |
| in DDL Cream | | |
| (Formulation 12) | | |
| Compound 1 0.3% | 7.32 | 12.0 |
| in DDL HPC Gel | | |
| (Formulation 11) | | |
| Compound 1 1.0% | 5.90 | 32.1 |
| in DDL HPC Gel | | |
| (Formulation 11) | | |

As evident from the results included in this example, the in vitro permeability test (IVPT) model did predict that dermal deposition directionality, i.e., that the gel formulations would have a higher dermal deposition as compared to the creams. However, IVPT did not predict the large quantitative difference observed in vivo at day 28 for the dermal deposition of Compound 1 in the minipig skin. However, as described in Table 12, the cream formulations have a higher systemic exposure of Compound 1 as compared to the gel formulations. This is unexpected and surprising because the gel formulations had a much higher delivered dose and a dermal deposition of Compound 1 in the IVPT evaluation.

Thus, the choice of excipients in designing the formulations is critical. The data from the experiment demonstrates that the surfactants/lipids in the cream formulation have a significant impact on the in vivo biopharmaceutics as compared to the results from the in vitro experiments.

### Example 8

### Dermal Rat Tolerability Screens:

The data provided below discloses the tolerability of various formulations of Compound 1. The formulations lacking Compound 1 (i.e., listed as 0% formulations) were positive controls for the experiments. The results were from a 14 day dermal rat tolerability study. The results of the experiments are disclosed in Table 13 below.

TABLE 13
Results from 14 day dermal rat tolerability screens:

| | | Erythema Score | | |
|---|---|---|---|---|
| Gp | Compound 1 Formulations | Rat #1 | Rat #2 | Rat #3 |
| 1* | Formulation 13 (F245) 0% Cream | 0 | 0 | 0 |
| 2* | Formulation 13 (F245) 1% Cream | 0 | 1→2 | 2→1 |
| 3 | Formulation 14(F249) 0% Cream | 2 | 1 | 1 |
| 4 | Formulation 14 (F249) 1% Cream | 2 | 2 | 3 ** |
| 5 | Formulation 12 (DDL) 0% Cream | 0 | 0 | 1 |
| 6 | Formulation 12 (DDL) 1% Cream | 0 | 0 | 0 |
| 7 | Formulation 11 (DDL) 0% HPC Gel | 0 | 0 | 0 |
| 8 | Formulation 11 (DDL) 1% HPC Gel | 0 | 0 | 0 |
| 9 | Formulation 9 (F156) 0% Cream | 2 | 2→1 | 2 |
| | (Positive Control) | | | |
| 10 | Formulation15 (DDL) 0% Carbopol | 0 | 0 | 0 |
| | Gel | | | |

Scoring chart: 0-no erythema; 1-very slight erythema (barely perceptible); 2-well-defined erythema; 3-moderate to severe erythema, 4-severe erythema (beet redness) to slight eschar formation (injuries in depth).

### Example 9

### Stability Studies

The physical solubility of the formulation is an important factor in the development of the formulation. In particular, F245 cream formulation, is physically unstable at 1% due to precipitation.

The active ingredient, Compound 1, is very stable, and has known stability of greater than 5 years. The formulations analyzed for stability exhibited good solubility characteristics when stored for 1 month at 50° C.

The stability studies were also conducted by storing the samples under the conditions of accelerated degradation (40° C./75% RH). Further data from forced degradation studies is provided below. The stress conditions and the resulting stability of the formulation is provided below. The results of the stability studies are provided in Table 14 below.

TABLE 14
Forced Degradation Results Summary for 0.1%
w/w FBM-DDL-HPC Gel formulations

| | | Compound 1 | Compound 1 |
|---|---|---|---|
| | | Degradation | Purity |
| Stress Condition | Exposure Time | (%) | Factor |
| Control | N/A | 0.0 | 999.99 |
| Heat, 80° C. | 7 Days | −0.2 | 1000.00 |
| 365 nm Light | 3 Days | 5.3 | 999.99 |
| 3% H₂O₂ | 3 Days | 0.9 | 1000.00 |
| 3% H₂O₂ & Heat, | 3 Day | 10.9 | 999.99 |
| 80° C. | | | |
| Acid, 0.1M HCl | 24 Hours | 1.0 | 999.99 |
| Base, 0.1M NaOH | 24 Hours | 0.6 | 999.98 |

The data from the experiments suggests that the gel formulations are stable under stressed conditions. The data indicates that the formulations may be sensitive to oxidation originating from peroxides and UV light irradiation.

### Example 10

Some of the exemplary formulations of the invention are provided below. Formulation 14 is a placebo, Formulation 15 comprises 0.1% (w/w) of Compound 1, Formulation 16 comprises 0.3% (w/w) of Compound 1, and Formulation 17 comprises 1.0% (w/w) of Compound 1.

TABLE 15
Formulations 13, 15, 16, and 17

| | | | Vehicle/ Placebo | | (0.1% w/w) | | (0.3% w/w) | | (1% w/w) | |
|---|---|---|---|---|---|---|---|---|---|---|
| | | | (Formulation 14) | | (Formulation 15) | | (Formulation 16) | | (Formulation 17) | |
| Composition | Grade | Function | % w/w | mg/g | % w/w | mg/g | % w/w | mg/g | % w/w | mg/g |
| Compound 1 | Pharm | Active drug | 0.0 | 0.0 | 0.1 | 1.0 | 0.3 | 3.0 | 1.0 | 10.0 |
| Diethylene | NF | Water | 46.00 | 460.00 | 46.00 | 460.00 | 46.00 | 460.00 | 46.00 | 460.00 |
| Glycol | | Soluble | | | | | | | | |
| Monoethyl | | Solvent, | | | | | | | | |
| Ether | | Enhancer | | | | | | | | |
| Alcohol 190 | USP | Water | 16.00 | 160.00 | 16.00 | 160.00 | 16.00 | 160.00 | 16.00 | 160.00 |
| Proof | | Soluble | | | | | | | | |
| | | Solvent | | | | | | | | |
| Propylene | USP | Water | 5.00 | 50.00 | 5.00 | 50.00 | 5.00 | 50.00 | 5.00 | 50.00 |
| Glycol | | Soluble | | | | | | | | |
| | | Solvent | | | | | | | | |
| Methylparaben | NF | Preservative | 0.20 | 2.00 | 0.20 | 2.00 | 0.20 | 2.00 | 0.20 | 2.00 |
| Propylparaben | NF | Preservative | 0.02 | 0.20 | 0.02 | 0.20 | 0.02 | 0.20 | 0.02 | 0.20 |
| Butylated | NF | Anti-oxidant | 0.20 | 2.00 | 0.20 | 2.00 | 0.20 | 2.00 | 0.20 | 2.00 |
| Hydroxytoluene | | | | | | | | | | |
| Sterile Water | NF | solvent | QSAD | QSAD | QSAD | QSAD | QSAD | QSAD | QSAD | QSAD |
| for Irrigation | | | 100 | 1000 | 100 | 1000 | 100 | 1000 | 100 | 1000 |
| | | | (30.58%) | (305.8 mg) | (30.48%) | (304.8 mg) | (30.28%) | (302.8 mg) | (29.58%) | (295.8 mg) |
| Hydroxypropyl | NF | Thickening | 2.00 | 20.00 | 2.00 | 20.00 | 2.00 | 20.00 | 2.00 | 20.00 |
| Cellulose | | Agent | | | | | | | | |

### Example 11

The Example provides further embodiments of the topical pharmaceutical compositions of the invention. Table 16 provides further exemplary embodiments.

TABLE 16
Formulations 16, 17, and 18

| | Compositions % (w/w) | | | |
|---|---|---|---|---|
| | | | Formulation | Formulation |
| | Formulation | Formulation | 18 | 18 |
| Ingredient | 16 (Gel) | 17(Gel) | (Cream) | (Cream) |
| Compound 1 | 0.30 | 1.00 | 0.30 | 1.00 |
| Diethylene Glycol | 46.00 | 46.00 | 46.00 | 46.00 |
| Monoethyl ether | | | | |
| Alcohol 190 Proof, | 16.00 | 16.00 | — | — |
| USP | | | | |
| Propylene glycol, | 5.00 | 5.00 | 5.00 | 5.00 |
| USP | | | | |
| Cyclomethicone | — | — | 1.00 | 1.00 |
| Methylparaben | 0.20 | 0.20 | 0.20 | 0.20 |
| Propylparaben | 0.02 | 0.02 | 0.02 | 0.02 |
| Butylated | 0.20 | 0.20 | 0.20 | 0.20 |
| Hydroxytoluene | | | | |
| Hydroxypropyl | 2.00 | 2.00 | — | — |
| cellulose | | | | |
| Polysorbate 80, | — | — | 2.00 | 2.00 |
| NF | | | | |
| Carbopol 980 | — | — | 0.40 | 0.40 |
| Pemulen | — | — | 0.40 | 0.40 |
| Petrolatum, USP | — | — | 13.00 | 13.00 |
| Arlacel 165 | — | — | 6.00 | 6.00 |
| 50% Trolamine | — | — | pH 5.5-6.0 | pH 5.5-6.0 |
| Solution | | | | |
| Sterile Water for | 30.28 | 29.58 | QS to 100% | QS to 100% |
| Irrigation | | | | |

### Example 12

The Example provides further embodiments of the topical pharmaceutical compositions of the invention. Formulations 19 and 20 are provided in Table 16 below.

TABLE 18
Formulations 19 and 20:

| | Compositions % (w/w) | |
|---|---|---|
| | Formulation 19 | Formulation 20 |
| Ingredient | (Cream) | (Cream) |
| Compound 1 | 0.30 | 1.00 |
| PEG-35 Castor Oil | 1.90 | 1.90 |
| PEG-60 Hydrogenated Castor Oil | 1.90 | 1.90 |
| Propylene Glycol Monolaurate (type II), | 3.00 | 3.00 |
| NF | | |
| Caprylic/Capric triglyceride, NF | 15.00 | 15.00 |
| 2-phenoxyethanol | 1.00 | 1.00 |
| Cyclomethicone, NF | 2.00 | 2.00 |
| Diethyl sebacate | 10.00 | 10.00 |
| Coconut Oil | 10.00 | 10.00 |
| Butylated Hydroxytoluene, NF | 0.10 | 0.10 |
| Stearyl Alcohol, NF | 5.00 | 5.00 |
| Glyceryl Stearate and PEG-75 Stearate | 7.00 | 7.00 |
| Dibasic sodium phosphate, dried, USP | 0.35 | 0.35 |
| Anhydrous Citric Acid, USP | 0.10 | 0.10 |
| Sterile Water for Irrigation | QS to 100% | QS to 100% |

### Example

### Effects of Topical Compound 1 Gel on Surgical Incision-Induced Dermal Fibrosis in Gottingen Minipigs

Minipig skin is more similar to human skin than rodent skin, and dermal delivery of topical ALK-5 kinase inhibitor Compound 1 in minipigs is expected to be reflective of treatment of human skin. Therefore, a model of surgical incision-induced dermal fibrosis was established in minipigs and the effects of topical Compound 1 were evaluated by visual assessment, histologic examination and gene expression by RNA sequencing.

The study was conducted in 4 female Göttingen SPF minipigs. On Day 1, eight full thickness incisional wounds, four on each side, were performed on each animal. Compound 1 0% (Vehicle), 0.1%, 0.3% or 1% was applied topically twice daily for 14 days from Day 7 through Day 20 on each incision. All animals received a dose volume of 160 μl per wound (5 L/cm2 applied to a 32 cm2 area surrounding each incision). Clinical signs, visual observations of incisions, body weight and food consumption were recorded during the in-life period. At necropsy (Day 21) two biopsies were collected from each incision for histopathology and gene expression analysis.

For histopathology, a 0.8 cm punch biopsy was collected from the incisional wounds. The biopsies were divided into two, diagonal to the incisional wound. All biopsies were individually fixed in phosphate buffered neutral 4% formaldehyde. Following fixation, all incisional wound biopsies collected for microscopic examination were trimmed and processed. The specimens were embedded in paraffin and cut at a nominal thickness of approximately 5 m, stained with haematoxylin and eosin, and examined by light microscopy.

For gene expression analysis, a 0.3 cm punch biopsy was collected from the incisional wounds. Samples were collected and stored overnight in prefilled tubes containing RNAlater at 5±3° C., and then transferred to a freezer and stored at ˜−20° C. The biopsies collected were then thawed and subjected to RNA purification and then generation of mRNA-enriched cDNA libraries for Illumina sequencing. RNA sequencing was performed to a depth of ˜15 million reads followed by bioinformatic analysis, including differential gene expression analysis.

No adverse clinical signs were recorded during the in-life period. Visual observation of the incisions indicated re-epithelialisation by Day 7. Histology evaluation indicated the presence of new collagen and complete re-epithelialisation indicating that wound healing was not compromised by Compound 1 and all incisions were in an advanced stage of healing by Day 21.

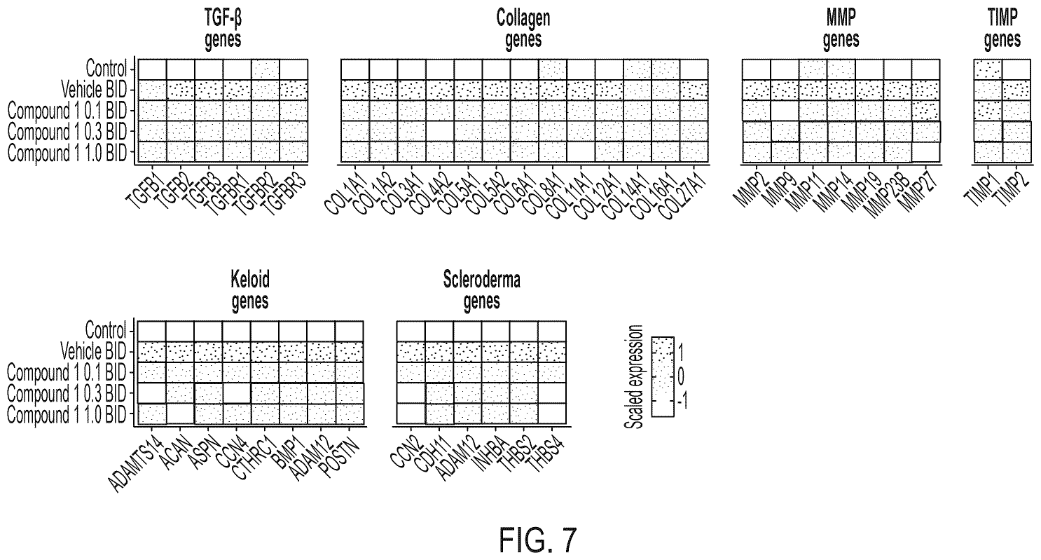

Compound 1 treatment exhibited a consistent tendency to dose-dependently reduce gene expression of the profibrotic genes investigated (FIG. 7). This includes the TGFβ ligands, TGF-β1, TGF-β2 and TGF-β3 by up to −23%, −32%, and −49%, respectively, multiple collagen genes COL1A1 (−49%), COL1A2 (−48%), COL3A1 (−49%), MMP genes MMP2 (−52%), MMP9 (−54%), MMP14 (−42%), TIMP genes TIMP1 (−17%) and TIMP2 (−46%), as well as multiple genes involved in fibrotic skin diseases such as keloid formation (Jumper et al., 2017) (e.g., ADAMTS14, −52%; BMP1, −47%), and in scleroderma patients with diffuse skin disease (Rice et al., 2015) (e.g. ADAM12, −52%; THBS4, −67%).

A total of 3256 genes were differentially expressed between normal tissue and vehicle-treated incisions (FIG. 7). Hierarchical clustering of all differentially expressed genes identified four distinct gene expression profiles, of which clusters 1 and 2 showed a tendency for dose-dependent effects of Compound 1 to inhibit fibrotic gene expression. Gene ontology pathway analysis on the genes in these clusters indicated a strong association with control of collagen metabolism, cell migration and motility, integrin-mediated signaling, and extracellular matrix organization.

Collectively, these gene expression and histology data demonstrate that topical administration of Compound 1 inhibits fibrosis in this minipig model of surgical incision-induced dermal fibrosis.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Combinations and Equivalents

All combinations of the various elements described herein are within the scope of the invention unless otherwise indicated herein or otherwise clearly contradicted by context.

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof

### REFERENCES CITED

Aarts P, Dudink K, Vossen A R J V, et al. (2021) Clinical Implementation of Biologics and Small Molecules in the Treatment of Hidradenitis Suppurativa. Drugs. 81:1397-1410. doi:10.1007/s40265-021-01566-2.
Aashaq S, Batool A, Mir S A, Beigh M A, Andrabi K I, Shah Z A (2022) TGF-β signaling: A recap of SMAD-independent and SMAD-dependent pathways. J Cell Physiol. 237(1):59-85.
Aguh C, Dina Y, Talbot C C Jr, Garza L. (2018) Fibroproliferative genes are preferentially expressed in central centrifugal cicatricial alopecia. J Am Acad Dermatol. November; 79(5):904-912.e1.
Allaway R J, Gosline S J C, La Rosa S, Knight P, Bakker A, Guinney J, Le L Q. (2018) Cutaneous neurofibromas in the genomics era: current understanding and open questions. Br J Cancer. 118(12):1539-1548. doi: 10.1038/s41416-018-0073-2. Epub 2018 Apr. 26. PMID: 29695767; PMCID: PMC6008439.
Amada H, Asanuma H, Koami T, et al. (2013) Discovery of 7-methoxy-6-[4-(4-methyl-1,3-thiazol-2-yl)-1H-imidazol-5-yl]-1,3-benzothiazole (TASP0382088): a potent and selective transforming growth factor-O type I receptor inhibitor as a topical drug for alopecia. Chem Pharm Bull (Tokyo). 61(3):286-291.
Anderson J L, Gutmann D H. Neurofibromatosis type 1. (2015) Handb Clin Neurol. 132:75-86. doi:10.1016/B978-0-444-62702-5.00004-4.
Asmaa M. El-Refaeya, Doaa M. El-Habak, Rana A. Khashaba, Reham Abd Al-mageed. (2020) Serum Levels of Transforming Growth Factor Beta1 (TGF β1) in Patients with Alopecia Areata. BMFJ 37(3):596-606.
Ayers N B, Sun C M, Chen S Y (2018) Transforming growth factor-O signaling in systemic sclerosis. J Biomed Res. 2018; 32(1):3-12.
Aykul S, Maust J, Thamilselvan V, Floer M, Martinez-Hackert E. (2021) Smad2/3 Activation Regulates Smad1/5/8 Signaling via a Negative Feedback Loop to Inhibit 3T3-L1 Adipogenesis. Int J Mol Sci. 6; 22(16):8472.
Baird K S, Crossan J F, Ralston S H. (1993) Abnormal growth factor and cytokine expression in Dupuytren's contracture. J Clin Pathol. 46(5):425-428. doi:10.1136/jcp.46.5.425.
Berndt A, Kosmehl H, Mandel U, et al. (1995) TGF beta and bFGF synthesis and localization in Dupuytren's disease (nodular palmar fibromatosis) relative to cellular activity, myofibroblast phenotype and oncofetal variants of fibronectin. Histochem J. 27(12):1014-1020.
Bian et al (2011) An ALK-5 inhibitor reduces collagen deposition in a rat dermal incision wound healing model. Plast Reconstr Surg. 128, 451e-459e.
Boys et al (2012) Discovery of a series of 2-(1H-pyrazol-1-yl) pyridines as ALK5 inhibitors with utility in dermal scarring. Bioorg Med Chem Lett. 22, 3392-3397.
Broekstra D C, Kuo R Y L, Burn E, Prieto-Alhambra D, Furniss D. (2023) Dupuytren Disease: Prevalence, Incidence, and Lifetime Risk of Surgical Intervention. A Population-Based Cohort Analysis. Plast Reconstr Surg. 151(3):581-591. doi:10.1097/PRS.0000000000009919.
Brosseau J P, Sathe A A, Wang Y, Nguyen T, Glass D A 2nd, Xing C, Le L Q. (2021) Human cutaneous neurofibroma matrisome revealed by single-cell RNA sequencing. Acta Neuropathol Commun. 9(1):11. doi: 10.1186/s40478-020-01103-4. PMID: 33413690; PMCID: PMC7792184.
Budi E H, Schaub J R, Decaris M, Turner S, Derynck R. (2021) TGF-β as a driver of fibrosis: physiological roles and therapeutic opportunities. J Pathol. 254(4):358-373.
Cannon A, Chen M J, Li P, et al. (2018) Cutaneous neurofibromas in Neurofibromatosis type I: a quantitative natural history study. Orphanet J Rare Dis. 13(1):31. Published 2018 Feb. 7. doi:10.1186/s13023-018-0772-z.
Chadha S A, Stout M A, Goyal P K, Nguyen C V, Lu K Q. (2023) Assessing unmet needs in patients with hidradenitis suppurativa. Arch Dermatol Res. 1-6. doi:10.1007/s00403-023-02645-y.
Chu S Y, Chen Y J, Tseng W C, Lin M W, Chen T J, Hwang C Y, Chen C C, Lee D D, Chang Y T, Wang W J, Liu H N. (2011) Comorbidity profiles among patients with alopecia areata: the importance of onset age, a nationwide population-based study. J Am Acad Dermatol. November; 65(5):949-56.
Chung E, De Young L, Brock G B. (2011) Rat as an animal model for Peyronie's disease research: a review of current methods and the peer-reviewed literature. Int J Impot Res. 23(6):235-241. doi:10.1038/ijir.2011.36.
Chung P H, Han T M, Rudnik B, Das A K. (2020) Peyronie's disease: what do we know and how do we treat it?. Can J Urol. 27(S3):11-19.
Clark K, Gak N, Orteu C H, Ong V H, Derrett-Smith E C, Denton C P. (2023) Skin biopsy analysis of concurrent keloidal morphoea and systemic sclerosis confirms overlapping pathogenic pathways [published online ahead of print, 2023 May 23]. Clin Exp Rheumatol. 2023; 10.55563/clinexprheumatol/dvxhgc. doi:10.55563/clinexprheumatol/dvxhgc.
Cui X, Shang S, Lv X, Zhao J, Qi Y, Liu Z (2019) Perspectives of small molecule inhibitors of activin receptor-like kinase in anti-tumor treatment and stem cell differentiation (Review). Mol Med Rep. 19(6):5053-5062.
Dadkhahfar S, Asadi Kani Z, Araghi F, Moravvej H. (2020) Development of keloidal morphea after treatment with cyclosporine in a case of recalcitrant generalized morphea. Clin Case Rep. 8(5):837-839. doi: 10.1002/ccr3.2776. PMID: 32477528; PMCID: PMC7250991.
Dajnoki Z, Somogyi O, Medgyesi B, Jenei A, Szabó L, Gáspár K, Hendrik Z, Gergely P, Imre D, Póliska S, Töröcsik D, Zouboulis C C, Prens E P, Kapitány A, Szegedi A. (2022) Primary alterations during the development of hidradenitis suppurativa. J Eur Acad Dermatol Venereol. 36(3):462-471. doi: 10.1111/jdv.17779.
Denton C P, Khanna D. (2017) Systemic sclerosis. Lancet. 7; 390(10103):1685-1699.
Díaz, D., Rivera, A., Otero, V. et al. (2022) Epidemiology of Hidradenitis Suppurativa: Current Status. Curr Derm Rep 11, 336-340. https://doi.org/10.1007/s13671-022-00372-7.
Dodagatta-Marri, E. et al (2019) α-PD-1 therapy elevates Treg/Th balance and increases tumor cell pSmad3 that are both targeted by α-TGFβ antibody to promote durable rejection and immunity in squamous cell carcinomas. Journal for ImmunoTherapy of Cancer 7, 62.
El-Aleem et al (2017) Co-upregulation of Transforming Growth Factor Beta-1 and Nitric Oxide Synthase in Keloid by Comparison to Normal Human Skin—A Possible Role for TGFβ1 and NOS in Pathogenesis of Keloid. J Cytol Histol. 8:4.
Fan Q, He M, Sheng T, Zhang X, Sinha M, Luxon B, Zhao X, Xie J. (2010) Requirement of TGFbeta signaling for SMO-mediated carcinogenesis. J Biol Chem. 285(47):36570-6.
Fernandes M S, Girisha B S, Viswanathan N, Sripathi H, Noronha T M. (2015) Discoid lupus erythematosus with squamous cell carcinoma: a case report and review of the literature in Indian patients. Lupus. 24(14):1562-1566. doi:10.1177/0961203315599245.
Feyisetan O. (2023) Peyronie's Disease: A Brief Overview. Cureus. 15(4):e37037. Published 2023 Apr. 2. doi:10.7759/cureus.37037.
Galoppini G, Marangoni A, Cirilli F, et al. (2023) Optimizing Patient Care: A Systematic Review of Multidisciplinary Approaches for SLE Management. J Clin Med. 12(12):4059. Published 2023 Jun. 15. doi:10.3390/jcm12124059.
Goldman N, Han J, LaChance A. (2022) Diagnosis and Management of Cutaneous Manifestations of Autoimmune Connective Tissue Diseases. Clin Cosmet Investig Dermatol. 2022 Oct. 26; 15:2285-2312. doi: 10.2147/CCID.S360801. PMID: 36320926; PMCID: PMC9618245.
Guiraud M, Bouroubi A, Beauchamp R, Bocquet A, Grégoire J M, Rauly-Lestienne I, Blanco I, Wolkenstein P, Schmitt A M. (2019) Cutaneous neurofibromas: patients' medical burden, current management and therapeutic expectations: results from an online European patient community survey. Orphanet J Rare Dis. 14(1):286. doi: 10.1186/s13023-019-1265-4. PMID: 31801570; PMCID: PMC6894276.
Gundogdu G, Nguyen T, Namasivayam A, Starek S, Gelman J, Mauney J R. (2023) Characterization of a novel rabbit model of Peyronie's disease [published online ahead of print, 2023 Feb. 13]. Int J Impot Res. 10.1038/s41443-023-00671-y. doi:10.1038/s41443-023-00671-y.
Guo L N, Perez-Chada L M, Borucki R, Nambudiri V E, Werth V P, Merola J F. (2021) Development of a working core outcome set for cutaneous lupus erythematosus: a practical approach to an urgent unmet need. Lupus Sci Med. 8(1):e000529. doi:10.1136/lupus-2021-000529.
Haag S M, Hauck E W, Szardening-Kirchner C, et al. (2007) Alterations in the transforming growth factor (TGF)-beta pathway as a potential factor in the pathogenesis of Peyronie's disease. Eur Urol. 2007; 51(1):255-261. doi:10.1016/j.eururo.2006.05.002.
Hahn et al (2013) Keloid-derived keratinocytes exhibit an abnormal gene expression profile consistent with a distinct causal role in keloid pathology. Wound Repair Regen. 21(4):530-44.
Hauck E W, Hauptmann A, Schmelz H U, Bein G, Weidner W, Hackstein H. (2003) Prospective analysis of single nucleotide polymorphisms of the transforming growth factor beta-1 gene in Peyronie's disease. J Urol. 2003; 169(1):369-372. doi:10.1016/S0022-5347(05)64129-8.
Hibino T, Nishiyama T. (2004) Role of TGF-beta2 in the human hair cycle. J Dermatol Sci. 35(1):9-18.
Jagadeesan et al. (2007) Transforming growth factor beta (TGFβ) and keloid disease, Int. J. Surg., 5(4), 278-285.
Jiang C, Kumar A, Yu Z, Shipman T, Wang Y, McKay R M, Xing C, Le L Q. (2023) Basement membrane proteins in extracellular matrix characterize NF1 neurofibroma development and response to MEK inhibitor. J Clin Invest. 2023 Jun. 15; 133(12):e168227. doi: 10.1172/JCI168227. PMID: 37140985; PMCID: PMC10266775.
Jumper N, Hodgkinson T, Paus R, et al. (2017) Site-specific gene expression profiling as a novel strategy for unravelling keloid disease pathobiology. PLOS ONE 2017; DOI:10.1371/journal.pone.0172955.
Karkampouna S, Kruithof B P, Kloen P, et al. (2014) Novel Ex Vivo Culture Method for the Study of Dupuytren's Disease: Effects of TGFβ Type 1 Receptor Modulation by Antisense Oligonucleotides. Mol Ther Nucleic Acids. 3(1):e142. Published 2014 Jan. 21. doi:10.1038/mtna.2013.69.
Katoulis A C, Christodoulou C, Liakou A I, et al. (2015) Quality of life and psychosocial impact of scarring and non-scarring alopecia in women. J Dtsch Dermatol Ges. 13(2):137-142.
Ke Y. & Wang, X.-J (2021) TGFβ Signaling in Photoaging and UV-Induced Skin Cancer. Journal of Investigative Dermatology 141, 1104-1110.
Krause C, Kloen P, Ten Dijke P. (2011) Elevated transforming growth factor R and mitogen-activated protein kinase pathways mediate fibrotic traits of Dupuytren's disease fibroblasts. Fibrogenesis Tissue Repair. 4(1):14. Published 2011 Jun. 28. doi:10.1186/1755-1536-4-14.
Kristina E N Clark and others, E079 Whole genome expression analysis from paired samples of keloidal morphea and systemic sclerosis skin identifies key scleroderma candidate genes, Rheumatology, Volume 58, Issue Supplement_3, April 2019, kez110.077, https://doi.org/10.1093/rheumatology/kez110.077.
Kuhn A, Landmann A. (2014) The classification and diagnosis of cutaneous lupus erythematosus. J Autoimmun. 48-49:14-19. doi:10.1016/j.jaut.2014.01.021.
Lambi A G, Popoff S N, Benhaim P, Barbe M F. (2023) Pharmacotherapies in Dupuytren Disease: Current and Novel Strategies [published online ahead of print, 2023 Mar. 17]. J Hand Surg Am. S0363-5023(23)00070-9. doi:10.1016/j.jhsa.2023.02.003.
Li X, Ding Z, Wu Z, Xu Y, Yao H, Lin K (2021) Targeting the TGF-β signaling pathway for fibrosis therapy: a patent review (2015-2020). Expert Opin Ther Pat. 31(8):723-743.
Liarte S, Bernabé-García Á, Nicolas F J. (2020) Role of TGF-β in Skin Chronic Wounds: A Keratinocyte Perspective. Cells. 2020 Jan. 28; 9(2):306.
Liu C, Li M K, Alster T S. (2021) Alternative Cosmetic and Medical Applications of Injectable Deoxycholic Acid: A Systematic Review. Dermatol Surg. 47(11):1466-1472.
Lomelí-Nieto J A, Muñoz-Valle J F, Baños-Hernández C J, et al (2022). Transforming growth factor beta isoforms and TGF-βR1 and TGF-βR2 expression in systemic sclerosis patients. Clin Exp Med. 2022; 10.1007/s10238-022-00841-0.
Luong, V. H., Chino T., Oyama, N. et al. (2018) Blockade of TGF-β/Smad signaling by the small compound HPH-15 ameliorates experimental skin fibrosis. Arthritis Res Ther. 20 (46).
Machado M O, Stergiopoulos V, Maes M, et al. Depression and Anxiety in Adults With Hidradenitis Suppurativa: A Systematic Review and Meta-analysis. (2019) JAMA Dermatol. 155 (8): 939-945. doi: 10.1001/jamadermatol.2019.0759.
Mariathasan, S. et al (2018) TGF-β attenuates tumor response to PD-L1 blockade by contributing to exclusion of T cells. Nature 554, 544-548.
Mirizio E, Liu C, Yan Q, Waltermire J, Mandel R, Schollaert K L, Konnikova L, Wang X, Chen W, Torok K S. (2021) Genetic Signatures From RNA Sequencing of Pediatric Localized Scleroderma Skin. Front Pediatr. 9:669116. doi: 10.3389/fped.2021.669116. PMID: 34164359; PMCID: PMC8215272.
Nanchahal J, Ball C, Davidson D, et al. (2018) Anti-Tumour Necrosis Factor Therapy for Dupuytren's Disease: A Randomised Dose Response Proof of Concept Phase 2a Clinical Trial. EBioMedicine. 33:282-288. doi: 10.1016/j.ebiom.2018.06.022.
Nanchahal J, Ball C, Rombach I, et al. (2022) Anti-tumour necrosis factor therapy for early-stage Dupuytren's disease (RIDD): a phase 2b, randomised, double-blind, placebo-controlled trial. Lancet Rheumatol. 4 (6): E407-E416. doi: 10.1016/S2665-9913 (22) 00093-5.
Napolitano M, Megna M, Timoshchuk E A, Patruno C, Balato N, Fabbrocini G, Monfrecola G. Hidradenitis suppurativa: from pathogenesis to diagnosis and treatment. (2017) Clin Cosmet Investig Dermatol. 10:105-115. doi: 10.2147/CCID.S111019.
Naruse T, Aoki M, Fujimoto N, et al. (2017) Novel ALK5 inhibitor TP0427736 reduces TGF-β induced growth inhibition in human outer root sheath cells and elongates anagen phase in mouse hair follicles. Pharmacol Rep. 69 (3): 485-491.
Nestor M, Berman B, Lu P, Molyneaux M. (2022) Safety and Efficacy of TGF-β1/COX-2 Silencing Therapeutic in Adults With Cutaneous Squamous Cell Carcinoma In Situ. J Drugs Dermatol. 21 (5): 472-477.
O'Kane and Ferguson, (1997) Int. J. Biochem. Cell Biol., 29:63-78.
Ostrowski K A, Gannon J R, Walsh T J. (2016) A review of the epidemiology and treatment of Peyronie's disease. Res Rep Urol. 8:61-70. Published 2016 Apr. 29. doi: 10.2147/RRU.S65620.
Peterson J M et al (2022) Galunisertib Exerts Antifibrotic Effects on TGF-β-Induced Fibroproliferative Dermal Fibroblasts. Int J Mol Sci. 15; 23 (12): 6689.
Piao S, Choi M J, Tumurbaatar M, et al. (2010) Transforming growth factor (TGF)-β type I receptor kinase (ALK5) inhibitor alleviates profibrotic TGF-β1 responses in fibroblasts derived from Peyronie's plaque. J Sex Med. 2010; 7 (10): 3385-3395. doi: 10.1111/j.1743-6109.2010.01753.x.
Poplausky D, Young J N, Tai H, Rivera-Oyola R, Gulati N, Brown R M. (2023) Dermatologic Manifestations of Neurofibromatosis Type 1 and Emerging Treatments. Cancers (Basel). 15(10): 2770. doi: 10.3390/cancers 15102770. PMID: 37345107; PMCID: PMC10216721.
Ratajczak-Wielgomas K, Gosk J, Rabczyński J, et al. (2012) Expression of MMP-2, TIMP-2, TGF-β1, and decorin in Dupuytren's contracture. Connect Tissue Res. 53 (6): 469-477. doi: 10.3109/03008207.2012.686542.
Rencic A, Brinster N, Nousari C H. (2003) Keloid morphea and nodular scleroderma: two distinct clinical variants of scleroderma?. J Cutan Med Surg. 7 (1): 20-24. doi: 10.1007/s10227-002-1138-6.
Rice L M, Padilla C M, McLaughlin S R, et al. (2015) Fresolimumab treatment decreases biomarkers and improves clinical symptoms in systemic sclerosis patients. J Clin Invest 125 (7): 2795-2807.
Rittes P G. The lipodissolve technique: clinical experience. (2009) Clin Plast Surg. 36 (2): 215-227.
Rodrigues J N, Becker G W, Ball C, et al. (2015) Surgery for Dupuytren's contracture of the fingers. Cochrane Database Syst Rev. (12): CD010143. Published 2015 Dec. 9. doi: 10.1002/14651858.CD010143.pub2.
Rosenberg J E, Ergun O, Hwang E C, et al. (2023) Non-surgical therapies for Peyronie's disease. Cochrane Database Syst Rev. 7 (7): CD012206. Published 2023 Jul. 17. doi: 10.1002/14651858.CD012206.pub2.
Ryu J K, Piao S, Shin H Y, et al. (2009) IN-1130, a novel transforming growth factor-beta type I receptor kinase (activin receptor-like kinase 5) inhibitor, promotes regression of fibrotic plaque and corrects penile curvature in a rat model of Peyronie's disease. J Sex Med. 6 (5): 1284-1296. doi: 10.1111/j.1743-6109.2009.01216.x.
Sandler A B, Scanaliato J P, Dennis T, et al. Treatment of Dupuytren's Contracture With Collagenase: A Systematic Review. Hand (N Y). 2022; 17 (5): 815-824. doi: 10.1177/1558944720974119.
Simakou T, Butcher J P, Reid S, Henriquez F L. (2019) Alopecia areata: A multifactorial autoimmune condition. J Autoimmun. 98:74-85.
Smith S D B, Okoye G A, Sokumbi O. (2022) Histopathology of Hidradenitis Suppurativa: A Systematic Review. Dermatopathology (Basel). 14; 9 (3): 251-257. doi: 10.3390/dermatopathology9030029.
Solé C, Gimenez-Barcons M, Ferrer B, Ordi-Ros J, Cortés-Hernández J. (2016) Microarray study reveals a transforming growth factor-β-dependent mechanism of fibrosis in discoid lupus erythematosus. Br J Dermatol. 2016; 175 (2): 302-313. doi: 10.1111/bjd.14539.
Song K M, Chung D Y, Choi M J, et al. (2020) Vactosertib, a Novel, Orally Bioavailable Activin Receptor-Like Kinase 5 Inhibitor, Promotes Regression of Fibrotic Plaques in a Rat Model of Peyronie's Disease. World J Mens Health. 38 (4): 552-563. doi: 10.5534/wjmh.190071.
Soreide E, Murad M H, Denbeigh J M, et al. (2018) Treatment of Dupuytren's contracture: a systematic review. Bone Joint J. 100-B (9): 1138-1145. doi: 10.1302/0301-620X.100B9.BJJ-2017-1194.R2.
Strömberg J, Vanek P, Fridén J, Aurell Y. Ultrasonographic examination of the ruptured cord after collagenase treatment or needle fasciotomy for Dupuytren's contracture. J Hand Surg Eur Vol. 2017; 42 (7): 683-688. doi: 10.1177/1753193417711594.
Tamashunas N L, Bergfeld W F. (2021) Male and female pattern hair loss: Treatable and worth treating. Clin J Med. 88 (3): 173-182.
Tauriello, D. V. F. et al (2018) TGFβ drives immune evasion in genetically reconstituted colon cancer metastasis. Nature 554, 538-543.
Taylor F L, Levine L A. (2007) Peyronie's Disease. Urol Clin North Am. 34 (4): 517-vi. doi: 10.1016/j.ucl.2007.08.017.
Terrier J E, Nelson C J. (2016) Psychological aspects of Peyronie's disease. Transl Androl Urol. 5(3): 290-295. doi: 10.21037/tau.2016.05.14.
Toyoda S, Shin J, Fukuhara A, Otsuki M, Shimomura I. (2022) Transforming growth factor β1 signaling links extracellular matrix remodeling to intracellular lipogenesis upon physiological feeding events. J Biol Chem. 298 (4): 101748.
Tsai Y C, Hung C Y, Tsai T F. Efficacy and Safety of Biologics and Small Molecules for Moderate-to-Severe Hidradenitis Suppurativa: A Systematic Review and Network Meta-Analysis. (2023) Pharmaceutics 15 (5): 1351. doi: 10.3390/pharmaceutics15051351.
Tse R, Howard J, Wu Y, Gan B S. Enhanced Dupuytren's disease fibroblast populated collagen lattice contraction is independent of endogenous active TGF-beta2. (2004) BMC Musculoskelet Disord. 5 (1): 41. Published 2004 Nov. 12. doi: 10.1186/1471-2474-5-41.
Tu W Z, Fu Y B, Xie X. (2019) RepSox, a small molecule inhibitor of the TGFβ receptor, induces brown adipogenesis and browning of white adipocytes. Acta Pharmacol Sin. 40 (12): 1523-1531.
Vander Ark A, Cao J, Li X. (2018) TGF-β receptors: In and beyond TGF-β signaling. Cell Signal 52:112-120.\
Vasquez R, Wang D, Tran Q P, et al. (2013) A multicentre, cross-sectional study on quality of life in patients with cutaneous lupus erythematosus. Br J Dermatol. 168 (1): 145-153. doi: 10.1111/j.1365-2133.2012.11106.x
Verjee L S, Verhoekx J S, Chan J K, et al. (2013) Unraveling the signaling pathways promoting fibrosis in Dupuytren's disease reveals TNF as a therapeutic target. Proc Natl Acad Sci USA. 110 (10): E928-E937. doi: 10.1073/pnas.1301100110.
Vossen ARJV, van der Zee H H, Prens E P. Hidradenitis Suppurativa: A Systematic Review Integrating Inflammatory Pathways into a Cohesive Pathogenic Model. (2018) Front Immunol. 14; 9:2965. doi: 10.3389/fimmu.2018.02965.
Walker D, Susa J S, Currimbhoy S, Jacobe H. (2017) Histopathological changes in morphea and their clinical correlates: Results from the Morphea in Adults and Children Cohort V. J Am Acad Dermatol. 76 (6): 1124-1130.
Wang X, Gu C, Shang F, Jin R, Zhou J, Gao Z. (2021) Inhibitory Effect of the LY2109761 on the Development of Human Keloid Fibroblasts. Anal Cell Pathol (Amst). 8883427.
Werth V P. Morphea. https://emedicine.medscape.com/article/1065782-overview #a5.
Wysong A. (2023) Squamous-Cell Carcinoma of the Skin. N Engl J Med. 15; 388 (24): 2262-2273.
Yamamoto T, Sawada Y, Katayama I, Nishioka K. (2005) Nodular scleroderma: increased expression of connective tissue growth factor. Dermatology. 211 (3): 218-223. doi: 10.1159/000087015.
Yao, C. et al (2020). 140 AP-1 and TGFβ cooperativity drives non-canonical Hedgehog signaling in resistant basal cell carcinoma. Journal of Investigative Dermatology. 140. S17.
Young A, Namas R, Dodge C, Khanna D. (2016) Hand Impairment in Systemic Sclerosis: Various Manifestations and Currently Available Treatment. Curr Treatm Opt Rheumatol. 2 (3): 252-269.
Zamani N, Brown C W. (2011) Emerging roles for the transforming growth factor-{beta} superfamily in regulating adiposity and energy expenditure. Endocr Rev. 32 (3): 387-403.
Zhang T, et al (2020) Current potential therapeutic strategies targeting the TGF-β/Smad signaling pathway to attenuate keloid and hypertrophic scar formation. Biomed Pharmacother. 129:110287.
U.S. Pat. No. 7,964,612
U.S. Pat. No. 8,455,512
U.S. Pat. No. 9,938,289
U.S. Pat. No. 9,090,625
U.S. Pat. No. 9,260,450

1. A pharmaceutical composition for topical application, the pharmaceutical composition comprising:
a therapeutically effective amount of an activin receptor-like kinase-5 (ALK-5) kinase inhibitor;
a permeation enhancer;
a solvent;
an antioxidant;
a thickening agent; and
a preservative.

2. The pharmaceutical composition of claim 1, wherein the pharmaceutical composition is a cream or a topical gel.

3-5. (canceled)

6. The pharmaceutical composition of claim 5, wherein the pharmaceutical composition comprises from about 0.05% (w/w) to about 1% (w/w) of the ALK-5 kinase inhibitor.

7. The pharmaceutical composition of claim 1, wherein the pharmaceutical composition has a slow in vivo dermal penetration rate enabling high absorption deposition into the skin tissues at steady state when the pharmaceutical composition is repeatedly applied to the skin.

8. The pharmaceutical composition of claim 7, wherein the pharmaceutical composition has a high dermal deposition rate but also reduces the systemic plasma exposure in the circulation of the ALK-5 kinase inhibitor.

9. (canceled)

10. The pharmaceutical composition of claim 1, wherein the thickening agent is selected from the group consisting of: carbomer, methyl cellulose, sodium carboxyl methyl cellulose (NaCMC), carrageenan, colloidal silicon dioxide, trolamine, guar gum, hydroxypropyl cellulose (HPC), hydroxypropyl methyl cellulose (HPMC), gelatin, polyethylene oxide, alginic acid, sodium alginate, fumed silica, and any combination thereof.

11. The pharmaceutical composition of claim 1, wherein the antioxidant is selected from a group a consisting of: butylated hydroxyanisole, butylated hydroxytoluene, tocopherol, propyl gallate, vitamin E, tert-butylhydroquinone and any combination thereof.

12. The pharmaceutical composition of claim 1, wherein the preservative is an antimicrobial preservative.

13. The pharmaceutical composition of claim 1, wherein the pharmaceutical composition does not include a preservative.

14. The pharmaceutical composition of claim 12, wherein the preservative is selected from a group consisting of: benzyl alcohol, imidazolidinyl urea, diazolidinyl urea, dichlorobenzyl alcohol, chloroxylenol, methyl paraben, ethyl paraben, propyl paraben, butyl paraben, phenoxyethanol, sorbic acid, benzoic acid, benzalkonium chloride, phenyl mercuric acetate, chlorobutanol, phenoxyethanol, and any combination thereof.

15. The pharmaceutical composition of claim 1, wherein the solvent is selected from a group consisting of water, hexylene glycol, propylene glycol, oleyl alcohol, propylene carbonate, mineral oil, diethylene glycol monoethyl ether, ethanol, polyethylene glycol, water, isopropanol, t-butyl alcohol, amyl alcohol, benzyl alcohol, diacetone alcohol, hexyl alcohol, tetrahydrofurfuryl alcohol, acetic acid, carboxylic acids, 1,2-hexanediol, butylene glycol, diethylene glycol, dipropylene glycol, ethyl hexanediol, ethylene glycol, propylene glycol monolaurate, tetraethylene glycol, triethylene glycol, tripropylene glycol, butyl stearate, C12-15 alkyl benzoate, C12-15 alkyl lactate, caprylic/capric triglyceride, cetearyl ethylhexanoate, diethylhexyl adipate, di-ethylhexyl succinate, diisopropyl adipate, dioctyl malate, di-PPG-2 myreth-10 adipate, di-PPG-3 myristyl ether adipate, ethyl oleate, ethylhexyl cocoate, ethylhexyl hydroxystearate, ethylhexyl palmitate, ethylhexyl pelargonate, ethylhexyl stearate, hexyl laurate, hexyldecyl laurate, stearic acid, isostearic acid and other long chain fatty acids, and any combination thereof.

16. The pharmaceutical composition of claim 1, wherein the permeation enhancer is selected from a group consisting of propylene glycol, ethanol, isopropyl alcohol, oleic acid, polyethylene glycol, diethylene glycol monoethyl ether, dimethyl sulfoxide, capric acid, hexanoic acid, lauric acid, linoleic acid, linolenic acid, propionic acid, and any combination thereof.

17. The pharmaceutical composition of claim 1, further comprising a surfactant.

18. The pharmaceutical composition of claim 17, wherein the surfactant is selected from a group consisting of polysorbate 80, pemulen TR-1, Arlacel 165, castor oil, hydrogenated castor oil, caprylic triglycerides, capric triglycerides, glycerol stearate, PEG sterate, and any combination thereof.

19. The pharmaceutical composition of claim 1, further comprising a glidant.

20. The pharmaceutical composition of claim 19, wherein the glidant is selected from a group consisting of silica, cyclomethicone, magnesium stearate, and any combination thereof.

21. The pharmaceutical composition of claim 1, further comprising a buffer.

22. The pharmaceutical composition of claim 21, wherein the buffer is phosphoric acid, citric acid, salts thereof, or any combination thereof.

23. The pharmaceutical composition of claim 1, further comprising excipients selected from group consisting of: petrolatum, diethyl sebacate, coconut oil, stearyl alcohol, and any combination thereof.

24. (canceled)

25. The pharmaceutical composition of claim 1, wherein the pharmaceutical composition comprises about 0.05% (w/w) to about 0.3% (w/w) of the ALK-5 kinase inhibitor.

26. The pharmaceutical composition of claim 1, wherein the pharmaceutical composition comprises about 0.08% (w/w) to about 0.3% (w/w) of the ALK-5 kinase inhibitor.

27. The pharmaceutical composition of claim 1, wherein the pharmaceutical composition comprises about 0.1% (w/w), about 0.3% (w/w), or about 1% (w/w) of the ALK-5 kinase inhibitor.

28. The pharmaceutical composition of claim 1, wherein the pharmaceutical composition comprises about 0.05% (w/w) to about 0.5% (w/w) of the preservative.

29. (canceled)

30. The pharmaceutical composition of claim 1, wherein the pharmaceutical composition comprises about 0.05% (w/w) to about 0.5% (w/w) of the antioxidant.

31. (canceled)

32. The pharmaceutical composition of claim 1, wherein the pharmaceutical composition comprises about 0.5% (w/w) to about 10% (w/w) of the thickener.

33. The pharmaceutical composition of claim 1, wherein the pharmaceutical composition comprises about 1% (w/w) to about 5% (w/w) of the thickener.

34. (canceled)

35. The pharmaceutical composition of claim 1, wherein the pharmaceutical composition comprises about 10% (w/w) to about 70% (w/w) of the permeation enhancer.

36. (canceled)

37. The pharmaceutical composition of claim 1, wherein the pharmaceutical composition comprises about 30% (w/w) to about 50% (w/w) of the permeation enhancer.

38. The pharmaceutical composition of claim 1, wherein the pharmaceutical composition comprises about 30% (w/w) to about 98% (w/w) of the solvent.

39. (canceled)

40. The pharmaceutical composition of claim 1, further comprising about 0.01% (w/w) to about 5% (w/w) of a pharmaceutically acceptable buffer.

41. (canceled)

42. The pharmaceutical composition of claim 1, wherein the pharmaceutical composition is designed for treatment of diseases or disorders selected from the group consisting of one or more of scars, hypertrophic scars, keloid scars, keloid morphea, skin fibrosis, scleroderma, skin cancer, basal cell carcinoma, squamous cell carcinoma, melanoma, cutaneous neurofibromas, cutaneous lupus erythematosus, discoid lupus erythematosus, hidradenitis suppurativa, dupuytrene's contracture and Peyronie's disease.

43. The pharmaceutical composition of claim 1, wherein the ALK-5 kinase inhibitor is selected from the group consisting of one or more of 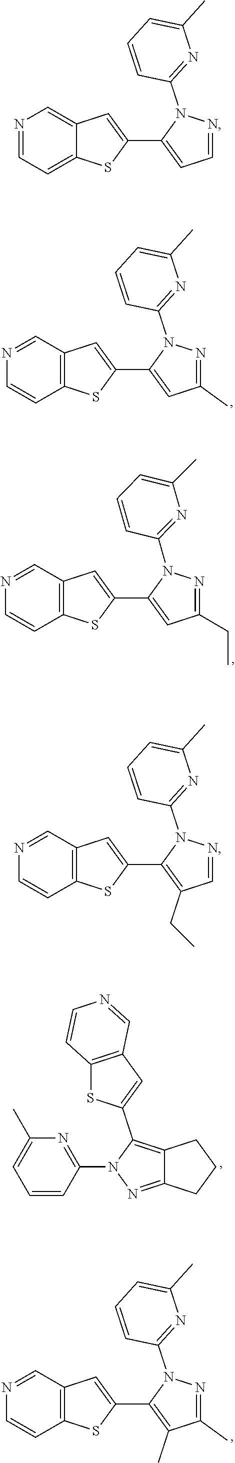 [SMILES: Cc1cccc(-n2nccc2-c2cc3cnccc3s2)n1 ; Cc1cccc(-n2nc(C)cc2-c2cc3cnccc3s2)n1 ; CCc1cc(-c2cc3cnccc3s2)n(-c2cccc(C)n2)n1 ; CCc1cnn(-c2cccc(C)n2)c1-c1cc2cnccc2s1 ; Cc1cccc(-n2nc3c(c2-c2cc4cnccc4s2)CCC3)n1 ; Cc1cccc(-n2nc(C)c(C)c2-c2cc3cnccc3s2)n1] 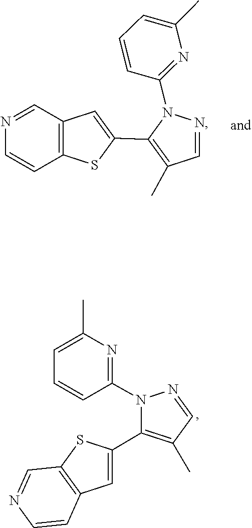 [SMILES: Cc1cccc(-n2ncc(C)c2-c2cc3cnccc3s2)n1 ; "and" ; Cc1cccc(-n2ncc(C)c2-c2cc3ccncc3s2)n1]
and a
pharmacologically acceptable salt thereof.

44. A process for preparing a pharmaceutical composition for topical application, the process comprising: mixing a solvent, an activin receptor-like kinase-5 (ALK-5) kinase inhibitor, a permeation enhancer, an antioxidant, a thickening agent, and optionally a preservative to thereby produce a pharmaceutical composition for topical application.

45. A method of treatment of a disease or disorder of the skin or affecting the skin by topical administration of a pharmaceutical composition to a patient, the pharmaceutical composition comprising:
an activin receptor-like kinase-5 (ALK-5) kinase inhibitor;
a permeation enhancer;
a solvent;
an antioxidant;
a thickening agent; and
optionally a preservative.

46-112. (canceled)
